(19) United States
(12) Patent Application Publication
(10) Pub. No.: US 2025/0235960 A1
(43) Pub. Date: Jul. 24, 2025

(54) METHODS FOR PERFORMING HIGH-SPEED, SCANNED LASER STRUCTURING OF MULTI-LAYERED ECO-BIORESORBABLE MATERIALS AND FABRICATING BIORESORBABLE ELECTRONIC DEVICES, AND APPLICATIONS THEREOF

(71) Applicant: NORTHWESTERN UNIVERSITY, Evanston, IL (US)

(72) Inventors: John A. Rogers, Wilmette, IL (US); Anthony Banks, Evanston, IL (US); Quansan Yang, Evanston, IL (US); Ziying Hu, Wilmette, IL (US)

(21) Appl. No.: 18/708,271

(22) PCT Filed: Nov. 10, 2022
(86) PCT No.: PCT/US2022/049522

§ 371 Date: May 8, 2024

(51) Int. Cl. B23K 26/402; A61B 5/00; A61B 5/026; B23K 26/362; H01L 21/268

(52) U.S. Cl. CPC B23K 26/402; A61B 5/686; B23K 26/362; H01L 21/268; A61B 5/026; A61B 2562/12

(57) ABSTRACT

This invention relates to methods for performing laser structuring of multi-layered ecoresorbable or bioresorbable materials and fabricating a bioresorbable electronic device using pisosecond-pulsed laser, devices fabricated by the methods, and applications of the same. Specifically, the method includes: sequentially forming a plurality of ecoresorbable or bioresorbable material layers on a flexible substrate; patterning, locally thinning or ablating, using a picosecond-pulsed laser system, the ecoresorbable or bioresorbable material layers to form sensing components and interconnection traces of the bioresorbable electronic device; and patterning and ablating, using a picosecond-pulsed laser system, the flexible substrate to form stretchable portions of the bioresorbable electronic device. The material layers may be formed on the flexible substrate by physical lamination, transfer printing, deposition or growth techniques. The use of the picosecond-pulsed laser is advantageous because the ablation process occurs on timescales sufficiently short to limit thermal diffusion and the associated spread of the heat-affected zone.

### CROSS-REFERENCE TO RELATED APPLICATIONS

This PCT application claims priority to and the benefit of U.S. Provisional Patent Application Ser. No. 63/277,664, which was filed Nov. 10, 2021. The content of the application is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### FIELD OF THE INVENTION

The present invention relates generally to healthcare and vital sign monitoring, and more particularly to methods for performing high-speed, scanned laser structuring of multi-layered eco/bioresorbable materials and fabricating bioresorbable electronic devices using picosecond-pulsed laser, and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Eco/bioresorbable electronic systems have a broad range of applications, from temporary biomedical implants to low-cost consumer gadgetry to internet-of-things (IoT) sensors, where processes of resorption eliminate risks and costs associated with surgical extraction and management of electronic waste. High-speed scalable manufacturing of such classes of electronics is a requirement for their widespread deployment in medical (e.g., millions of registered cardiac pacemakers and brain monitors worldwide) or IoT (over 20 billion worldwide, including 2 billion radio-frequency identification (RFID) tags specifically) devices. Published examples rely on multi-step processes that combine photolithography-based methods in microfabrication with transfer printing, designed to avoid chemical or thermal degradation of the eco/bioresorbable constituent materials. These schemes work well for demonstrations but might present challenges in cost-effective, mass production. Alternatives that exploit solution-based additive printing of functional inks can form simple, low-resolution passive components, but do not apply effectively to high-performance eco/bioresorbable semiconductors such as monocrystalline silicon.

Therefore, a heretofore unaddressed need exists in the art to address the aforementioned deficiencies and inadequacies.

### SUMMARY OF THE INVENTION

In one aspect, the invention relates to a method for method for performing laser structuring of multi-layered ecoresorbable or bioresorbable materials. In certain embodiments, the method includes: sequentially forming a plurality of ecoresorbable or bioresorbable material layers on a flexible substrate; patterning, locally thinning or ablating, using a picosecond-pulsed laser system, the ecoresorbable or bioresorbable material layers; and patterning and ablating, using the picosecond-pulsed laser system, the flexible substrate.

In one embodiment, the flexible substrate is a flexible biodegradable polymeric substrate. In one embodiment, the flexible substrate is formed by polylactic acid (PLA) or cellulose acetate (CA).

In one embodiment, the material layers are formed on the flexible substrate by physical lamination, transfer printing, deposition or growth techniques.

In one embodiment, the picosecond-pulsed laser system is operated with a wavelength of about 1030 nm, a pulse duration of about 1.0 picoseconds, and a beam diameter of 15 μm.

In one embodiment, the material layers include a semiconductor layer and a metal layer.

In one embodiment, the semiconductor layer and the metal layer are formed and patterned by: forming the semiconductor layer on the flexible substrate; patterning, locally thinning or ablating, using the picosecond-pulsed laser system, the semiconductor layer; after patterning, locally thinning or ablating the semiconductor layer, forming the metal layer on the flexible substrate and the patterned semiconductor layer; and patterning, locally thinning or ablating, using the picosecond-pulsed laser system, the metal layer.

In one embodiment, the method further includes: after patterning, locally thinning or ablating the semiconductor layer and prior to forming the metal layer, forming at least one alignment marker on the semiconductor layer to align the patterned semiconductor layer and the metal layer.

In one embodiment, a patterned structure of the ecoresorbable or bioresorbable material layers has a resolution of about 5-10 μm and alignment accuracy of less than 5 μm.

Another aspect of the invention relates to a method for fabricating a multi-layered bioresorbable electronic device. In certain embodiments, the method includes: sequentially forming a plurality of ecoresorbable or bioresorbable material layers on a flexible substrate; and patterning, locally thinning or ablating, using a picosecond-pulsed laser system, the ecoresorbable or bioresorbable material layers to form at least one sensing component and interconnection traces of the bioresorbable electronic device.

In one embodiment, the flexible substrate is a flexible biodegradable polymeric substrate. In one embodiment, the flexible substrate is formed by polylactic acid (PLA) or cellulose acetate (CA).

In one embodiment, the material layers are formed on the flexible substrate by physical lamination, transfer printing, deposition or growth techniques.

In one embodiment, the picosecond-pulsed laser system is operated with a wavelength of about 1030 nm, a pulse duration of about 1.0 picoseconds, and a beam diameter of 15 μm.

In one embodiment, the bioresorbable electronic device is a bi-layered electronic device, and the material layers include a semiconductor layer and a metal layer.

In one embodiment, the semiconductor layer and the metal layer are formed and patterned by: forming the semiconductor layer on the flexible substrate; patterning, locally thinning or ablating, using the picosecond-pulsed laser system, the semiconductor layer to form the at least one sensing component of the bioresorbable electronic device; after patterning, locally thinning or ablating the semiconductor layer, forming the metal layer on the flexible substrate and the patterned semiconductor layer; and patterning, locally thinning or ablating, using the picosecond-pulsed laser system, the metal layer to form the interconnection traces of the bioresorbable electronic device.

In one embodiment, the method further includes: after patterning, locally thinning or ablating the semiconductor layer and prior to forming the metal layer, forming at least one alignment marker on the semiconductor layer to align the patterned semiconductor layer and the metal layer.

In one embodiment, the bioresorbable electronic device is a multi-layered electronic device, and the material layers includes a first sensing layer and a plurality of second sensing layers.

In one embodiment, the first sensing layer and the second sensing layers are formed and patterned by: forming the first sensing layer on the flexible substrate; patterning, locally thinning or ablating, using the picosecond-pulsed laser system, the first sensing layer; and after patterning the first sensing layer, sequentially forming and patterning, locally thinning or ablating each of the second sensing layers by: forming a respective second sensing layer on the flexible biodegradable polymer substrate and the patterned first sensing layer; and patterning, locally thinning or ablating, using the picosecond-pulsed laser system, the respective second sensing layer.

In one embodiment, the first sensing layer and the second sensing layers include at least one semiconductor layer and at least one metal layer.

In one embodiment, the method further includes: after patterning, locally thinning or ablating the semiconductor layer and prior to forming the metal layer, forming at least one alignment marker on the semiconductor layer to align the patterned semiconductor layer and the metal layer.

In one embodiment, the method further includes: patterning and ablating, using the picosecond-pulsed laser system, the flexible substrate around the at least one sensing component and the connection traces to form at least one stretchable portion of the bioresorbable electronic device

A further aspect of the invention relates to a method for fabricating a multi-layered bioresorbable electronic device. In certain embodiments, the method includes: forming a first material layer on a flexible substrate, wherein the first material layer is ecoresorbable or bioresorbable; patterning, locally thinning or ablating, using a picosecond-pulsed laser system, the first material layer to form at least one sensing component of the bioresorbable electronic device; after patterning, locally thinning or ablating the first material layer, forming a second material layer on the flexible substrate and the patterned first material layer, wherein the second material layer is ecoresorbable or bioresorbable; and patterning, locally thinning or ablating, using the picosecond-pulsed laser system, the second material layer to form interconnection traces of the bioresorbable electronic device.

In one embodiment, the flexible substrate is a flexible biodegradable polymeric substrate. In one embodiment, the flexible substrate is formed by polylactic acid (PLA) or cellulose acetate (CA).

In one embodiment, the material layers are formed on the flexible substrate by physical lamination, transfer printing, deposition or growth techniques.

In one embodiment, the picosecond-pulsed laser system is operated with a wavelength of about 1030 nm, a pulse duration of about 1.0 picoseconds, and a beam diameter of 15 μm.

In one embodiment, the first material layer is a semiconductor layer, and the second material layer is a metal layer.

In one embodiment, the method further includes: after patterning, locally thinning or ablating the first material layer and prior to forming the second material layer, forming at least one alignment marker on the first material layer to align the patterned first material layer and the second material layer.

In one embodiment, the method further includes: patterning and ablating, using the picosecond-pulsed laser system, the flexible substrate around the at least one sensing component and the connection traces to form at least one stretchable portion of the bioresorbable electronic device.

In certain embodiments, the methods described above have a manufacture time of about 30 minutes.

Yet a further aspect of the invention relates to a bioresorbable electronic device formed by the method as discussed above, or an electronic apparatus having a multi-layer bioresorbable electronic device formed by the method discussed above. In certain embodiments, the bioresorbable electronic device has a resolution of about 5-10 μm and alignment accuracy of less than 5 μm.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIG. 1A shows a flowchart of a method for performing laser structuring of multi-layered ecoresorbable or bioresorbable materials according to certain embodiments of the invention.

FIG. 1B shows a flowchart of a method for fabricating a multi-layered bioresorbable electronic device according to certain embodiments of the invention.

FIG. 2A schematically shows (a)-(c) an exemplary process of fabricating a bioresorbable electronic device using ultrashort-pulsed laser according to certain embodiments of the invention; and (d) representative electronic devices according to certain embodiments of the invention.

FIG. 2B schematically shows cross-sectional views of laser ablation procedures for a bioresorbable electronic device using ultrashort-pulsed laser according to certain embodiments of the invention.

FIG. 2C schematically shows examples of bioresorbable electronic devices being fabricated according to certain embodiments of the invention.

FIG. 3 schematically shows a comparison of the current fabrication methods of a bioresorbable electronic device and the method for fabricating bioresorbable electronic device using ultrashort-pulsed laser according to certain embodiments of the invention.

FIG. 4A shows a table of comparison between the method for fabricating a bioresorbable electronic device using ultrashort-pulsed laser according to certain embodiments of the invention and existing technologies.

FIG. 4B schematically shows representative laser-processed bioresorbable devices according to certain embodiments of the invention.

FIG. 5 shows controlled reductions in thicknesses of monocrystalline Si MMs achieved by ablation, and their dependence on ablation parameters, including (a) average power, (b) scanning speed and frequency, and (c) number of repetitions at fixed grid distance (7 μm) and grid mode (XY-parallel) according to certain embodiments of the invention.

FIG. 6 shows simulation results for controlled thickness reduction monocrystalline Si MM by tuning the laser parameters, and their dependence on ablation parameters, including (a) average power, (b) scanning speed and frequency, and (c) number of repetitions according to certain embodiments of the invention.

FIG. 7 shows experimental and simulation results for controlled thickness reduction in Mg by tuning the laser parameters, and their dependence on ablation parameters, including (a) average power, (b) scanning speed and frequency, and (c) number of repetitions according to certain embodiments of the invention.

FIG. 8 schematically shows illustrations of key parameters used for ablation using ultrashort pulsed lasers according to certain embodiments of the invention.

FIG. 9 shows the method resolution in thickness reduction (˜35 nm) according to certain embodiments of the invention.

FIG. 10 shows simulations of the thermal diffusion zone induced by a body heat flux with durations t from 10 ns to 100 fs according to certain embodiments of the invention, where (a) shows normalized temperature distribution on a Si/PLA (thickness: 500 nm/50 μm) structure at time t, (b) shows normalized temperature as a function of the distance to the edge of the heat flux region for different durations, and (c) shows length scale values with different durations, which highlights that the ultrashort pulsed laser efficiently minimizes the thermal diffusion zone adjacent to the edges of patterned features.

FIG. 11 schematically shows different views of Gaussian distribution of the power density along the radial direction of the laser spot (diameter: ˜15 μm) according to certain embodiments of the invention.

FIG. 12 shows schematic views of cross-sectional profiles of ribbon-shaped structures of monocrystalline Si MM formed by ablation in different cases according to certain embodiments of the invention, where (a) shows a trapezoid shape with a titled edge (projected width larger than the laser spot diameter Dₗₐₛₑᵣ in case 1, (b) shows a triangle shape at the critical point (projected width similar to Dₗₐₛₑᵣ in case 2, and (c) shows a proportionally reduced triangle shape (projected width smaller than Dₗₐₛₑᵣ in case 3; (d) shows experimentally measured profiles for these three cases; and (e) peak height and (f) effective width as functions of projected width.

FIG. 13 shows simulation results for the cross-sectional profiles of Si ribbons patterned in three cases as shown in FIG. 12(d).

FIG. 14 shows characteristic feature sizes of a monocrystalline Si ribbon patterned on a PLA substrate according to certain embodiments of the invention, where (a) shows an optical image of the Si ribbon on a PLA substrate (average power: 90 mW; scanning speed: 300 mm s−2; frequency: 200 kHz; number of repetition: 1; grid distance: 1 μm; grid mode: Y-parallel; projected width: 8 μm), (b) shows SEM characterization of the Si ribbon with inset showing high-magnification SEM image of the Si ribbon, (c) shows SEM analysis indicates that the average ribbon width and the RMS edge roughness are 3.5±0.7 and 1.1±0.4 μm, respectively, (d) shows AFM characterization of the Si ribbon, and (e) shows AFM analysis reveals that the full width at half maximum of the ribbon is ˜4 μm.

FIG. 15 shows quantitative characterization of a trench shape formed by the laser method according to certain embodiments of the invention, where (a) shows cross-sectional area, (b) shows peak height, and (c) shows effective width as functions of projected width. The minimum feature size is ˜5 μm. The shaded areas denote the standard deviation.

FIG. 16 shows (a) alignment accuracy in x-and y-axis is 2.7±1.3 and 3.1±1.2 μm, respectively, (b) minimized damage to underlying materials following laser ablation processing of the top layer, ablating a 500-nm-thick top Si MM layer leads to an ablated thickness of ˜80 nm for the underlying PLA layer (original thickness: 50 μm); ablating a 300-nm-thick top Mg layer leads to an ablated thickness of ˜50 nm for the underlying Si layer (original thickness: 500 nm), and (c) root-mean-square (RMS) line edge roughness for Si (thickness: 2 μm; width: 100 μm) and Mg (thickness: 500 nm; width: 100 μm) ribbons patterned on PLA substrates (thickness: 50 μm) are 1.1±0.4 μm and 0.8±0.2 μm, respectively, and insets showing SEM images (titled angle: 45°) of a bi-layer Mg/Si structure (thickness: 300 nm for top Mg and 2 μm for bottom Si) on a PLA substrate (thickness: 50 μm).

FIG. 17 shows a schematic view and an optical micrograph of the pattern for characterizing overlay registration, corresponding to FIG. 16(a). The distance between two crisscross patterns is designed to 500 μm. The difference between the actual measured distance and the designed distance determines the overlay registration.

FIG. 18 shows the laser ablation method for surface treatment on substrates according to certain embodiments of the invention, where (a) shows laser-treated micro-textured PLA surface (right) compared with pristine PLA surface (left), (b) and (c) show cross-sectional images of a water droplet (volume: 1.5 μL) on (b) the pristine PLA surface (c) laser-treated micro-textured PLA surface, and (d) shows contact angle values of the pristine and laser-treated surfaces are 67±1° and 97±2°, respectively. The laser treatment enhances surface hydrophobicity.

FIG. 19 shows SEM characterization of laser-ablated Si and Mg structures according to certain embodiments of the invention, where (a) shows SEM images (tilted angle: 75°) of an ablated Si ribbon (width: 50 μm; thickness: 2 μm) on a PLA substrate (thickness: 50 μm), (b) shows SEM images (tilted angle: 75°) of an ablated Mg ribbon (width: 50 μm; thickness: 500 nm) on a PLA substrate (thickness: 50 μm), and (c) shows SEM images (titled angle: 45°) of a bi-layer Mg/Si structure (thickness: 300 nm for top Mg and 2 μm for bottom Si) on a PLA substrate (thickness: 50 μm).

FIG. 20 shows capability for forming arrays of devices over large areas according to certain embodiments of the invention, including optical micrographs of an array (materials: Mg/PLA;

thickness: 0.3/50 μm; dimension: 85×55 mm) of 45 thin-film resistive-type temperature sensors (arrangement: 9×5), on a serpentine-type substrate of PLA, to allow stretchability.

FIG. 21 shows the strategy for thinning material structures used in wireless, bioresorbable physiological monitors according to certain embodiments of the invention, where (a) shows the process applied to an inductor with a thinned bottom electrode, as a key component in such a device, (b) schematically shows the devices formed in this manner, (c) shows a photograph of an inductor with thinned bottom electrode on a PLA substrate, (d) shows the height profile of the key component determined by 3D confocal optical microscopy, (e) shows the quantitative results along the white dashed line in (d), and (f) shows the working principle of such a device.

FIG. 22 shows laser ablation strategy for bioresorbable inductors at the cm-and sub-mm scales according to certain embodiments of the invention, where (a) shows BTP adhesive enables robust lamination of the metal layer on a polymer substrate by thermal compression, (b) shows the Mg inductor presents a resonance frequency of ˜175 MHz with a Q factor larger than 70, (c) shows a zoom-in image in sub-mm scale (diameter: ˜1 mm; number of turns: 4; line width: 90 μm; spacing: 20 μm), and (d) shows the zoom-in image in cm-scale.

FIG. 23 shows in vivo acute evaluations in a rat model according to certain embodiments of the invention, where (a) shows a photograph of a bioresorbable physiological monitoring device mounted above an opened craniotomy, with a standard clinical ICP monitor as a reference, and (b)-(d) show acute recordings of physiological parameters, including (b) ICP (red: bioresorbable device; blue: commercial reference), (c) respiration rate and (d) heart rate in the rat model.

FIG. 24 shows laser thinning strategy for bioresorbable resistive-type devices according to certain embodiments of the invention, where (a) shows the laser thinning method reduces the thickness of the device sensing area from 25 to ˜5-6 μm for high resistance and high sensitivity, while leaving the thicknesses of the connection traces unchanged (thickness: 25 μm), and (b) shows the resistance of the entire device increases by ˜3.5 times when the thickness of the sensing area is reduced from 25 to ˜5-6 μm.

FIG. 25 shows laser patterning of (a) Zn and (b) Mo on CA substrates into flexible sensing arrays according to certain embodiments of the invention.

FIG. 26 shows laser thinning and cutting of non-eco/bioresorbable polymers, including polyimide (PI) and polydimethylsiloxane (PDMS), into probe shapes according to certain embodiments of the invention.

FIG. 27 shows the process for fabricating bioresorbable microvascular flow sensing probes according to certain embodiments of the invention, where (a) shows the process including (1) vacuum deposition of a uniform Mg layer (thickness: 180 nm) on a PLA substrate (thickness: 50 μm), (2) ablation of the Mg layer to define four resistive-type devices, (3) and ablation of the PLA substrate to define a narrow, needle-shaped geometry, (b) shows an optical micrograph of such a device, with a magnified image in the inset to highlight the structures, (c) shows temperature distribution around the probe, where an input power of 33 mW generates a maximum temperature increase of ˜16° C. in air, (d) shows temperature distribution along with the flow probe at different heater powers, and (e) shows the relationship between the resistance of the thermistor and temperature is linear over this range, with a sensitivity of ˜3.3 Ω° C.⁻¹.

FIG. 28 shows bioresorption of the laser-fabricated devices according to certain embodiments of the invention.

FIG. 29 shows in vivo acute evaluations in a porcine model using a rectus abdominus myocutaneous flap according to certain embodiments of the invention, where (a) shows an optical image of the left rectus abdominus flap of a porcine model, (b) shows StO₂ status of the flap measured by a commercial reference device for different situations (R: release, no occlusion; I: ischemia, artery occlusion; C: congestion, vein occlusion), (c) shows the temperature difference between two thermistors measured by the bioresorbable device in these situations during operation of the heater, where the temperature differences are ˜0.35° C. in the R state, ˜0.45° C. in the I and C states, respectively, and (d) shows corresponding microcapillary flow rate of the flap determined from these data for each situation, where the microcapillary flow rates are 0.8±0.2 mm s⁻¹ in the R state, 0.2±0.1 mm s⁻¹ in the I state, and 0.05±0.02 mm s⁻¹ in the C state, respectively.

FIG. 30 shows biocompatibility of bioresorbable sensing probes formed by laser ablation according to certain embodiments of the invention, where H&E staining of tissue from the leg of a rat model (a) 4 weeks and (b) 8 weeks after implantation.

FIG. 31 shows laser ablation strategy for Si-based resistive-type devices according to certain embodiments of the invention, where (a) shows an optical micrograph of pristine monocrystalline Si (thickness: 2 μm) on a CA substrate (thickness: 35 μm), and (b) shows an optical micrograph of the ablated monocrystalline Si.

FIG. 32 shows laser ablation strategy for forming Si-based capacitive-type devices according to certain embodiments of the invention, where (a) shows an optical micrograph of the interdigitated structure, and (b) shows a magnified image thereof (line width: 100 μm).

FIG. 33 shows multi-layer fabrication strategies for Si-based devices and Si-based electrode arrays according to certain embodiments of the invention, where (a) shows the process to form the multi-layer device, including (1) transfer printing of a monocrystalline highly n-doped Si MM (thickness: 2 μm) on a PLA substrate (thickness: 50 μm), (2) ablation to pattern the Si MM into an array of electrodes, (3) vacuum deposition of a uniform Mg layer (thickness: 1 μm), and (4) ablation of the Mg layer to define connection traces and to cut the PLA substrate into a ribbon shape, (b) shows an optical micrograph of such a device, with 12 Si electrodes and Mg traces, and (c) and (d) show electrochemical impedance spectrum (where (c) shows impedance and (d) shows phase angle) of three representative electrodes measured in PBS (pH 7.4) at room temperature (dash line).

FIG. 34 shows an alternative process for forming multi-layer Si-based electrode arrays according to certain embodiments of the invention, including (1) transfer printing of a monocrystalline highly n-doped Si MM (thickness: 2 μm) on a PLA substrate (thickness: 50 μm), (2) vacuum depositing a uniform layer of Mg (thickness: 1 μm), (3) ablating the Mg layer to define connection traces, and (4) ablating the Si MM to define an array of electrodes and to cut the PLA substrate into a ribbon shape.

FIG. 35 shows an equivalent circuit model used to fit the electrochemical impedance spectra of Si-based electrodes according to certain embodiments of the invention, where the interface charge transfer resistance, R_{ct}, and double layer capacitance per unit area, C_{dl}, are ˜250 MΩ and 2.8 μF cm⁻², respectively.

FIG. 36 shows in vivo acute optogenetic evaluations in a mouse model according to certain embodiments of the invention, where (a) and (b) show in vivo ECoG recordings in a mouse measured by a representative Si electrode (a) without and (b) with optical simulation.

FIG. 37 shows multi-layer laser ablation strategy for Si-based temperature sensing arrays according to certain embodiments of the invention, where (a)-(c) show optical micrographs of the device, including four temperature sensors, with highly p-doped monocrystalline Si (thickness: 1 μm) as the sensing element and Mg (thickness: 500 nm) as interconnection traces, and (d) shows resistance-temperature relation of such a device indicates a sensitivity of ˜17.1 Ω° C.⁻¹.

FIG. 38 shows Si-based MOSFETs and diodes according to certain embodiments of the invention, where (a) shows the process to form a Si-based n-channel MOSFET, including (1) doping of specific regions of a Si MM (thickness: 2 μm), (2) ablation to pattern the Si MM into a rectangular pattern, (3) vacuum deposition of a uniform SiO₂ dielectric layer (thickness: 100 nm), (4) ablation of the SiO₂ layer to define shapes similar to those of Si and with two openings for metal interconnection, (5) vacuum deposition of a uniform Mg layer (thickness: 50 nm), and (6) ablation of the Mg layer to define three connection pads as electrodes, (b)-(c) show (b) transfer characteristics and (c) output characteristics of bioresorbable n-channel MOSFETs formed by ablation (solid line) and previously reported approaches (dash line) with the same materials and dimensions, and (d) shows IV characteristics of bioresorbable Si diodes with the same materials and the same dimensions fabricated by ablation (solid line) and previously reported approaches (dash line) in linear and log scale.

FIG. 39 shows (a) a top view, (b) a cross-sectional view, (c) a 3D exploded view, and (d) an optical micrograph for the multi-layer Si-based n-channel MOSFET devices fabricated by laser methods corresponding to FIG. 38.

FIG. 40 shows multi-layer laser ablation strategy for Si-based diodes according to certain embodiments of the invention, where (a) shows the laser ablation process to form a multi-layer Si-based diode, including (1) doping a specific region (dimension of highly n-doped area: 500×500 μm) on a monocrystalline Si MM (thickness: 2 μm), (2) ablation to pattern the Si MM into a rectangular pattern (dimension: 500×800 μm) into the PN junction pattern, (3) vacuum deposition of a uniform SiNx dielectric layer (thickness: 200 nm), (4) ablation of the SiNx layer to define shapes similar to those of Si and with two exposed vias (dimension: 50×140 μm), (5) vacuum deposition of a uniform Mg layer (thickness: 100 nm), and (6) ablation of the Mg layer to define two connection pads (dimension: 155×280 μm), (b) shows a top view of the diode fabricated by laser ablation, (c) shows a cross-sectional view thereof, and (d) shows a 3D exploded view thereof.

FIG. 41 shows (a) an optical micrograph of the diode fabricated by laser ablation as shown in FIG. 40, and (b)-(c) show photoresponse characteristics thereof, where the intensity of the white LED used in the light environment is 10,000 lux.

FIG. 42 shows laser ablation for integrated wireless bioresorbable cardiac systems according to certain embodiments of the invention, where (a) shows the components used to construct the cardiac devices, (b) shows a photograph of the multi-sensing element, (c) shows a photograph of the flexible cable, (d) shows a photograph of the wireless module, (e) shows the working principle of the device, and (f) shows a photograph of the complete device for measuring strains on the surface of cardiac tissue in large animals.

FIG. 43 shows device architectures and working principles of the multi-sensing element as shown in FIG. 42, where (a) shows multi-sensing element connects to the wireless module by a long flexible cable, (b) shows the element measures strains along the three arms, (c) shows an exploded view of the element, including three functional layers (bottom electrodes, floating electrodes, and top cover) and two encapsulation layers, and (d) shows the working principles for strain sensing.

FIG. 44 shows expansion and contraction of the heart during each cardiac cycle leads to epicardial strains that can be measured by the multi-sensing element according to certain embodiments of the invention.

FIG. 45 shows tissue-like mechanical and water barrier encapsulation properties of the WPU layer according to certain embodiments of the invention, where (a) shows the stress-strain curve for the WPU indicates a tissue-like Young's modulus of ˜100 kPa, (b) shows relative water permeation properties of WPU and pure PU, determined by the time dependence of the resistance of a thin-film Mg trace, and (c)-(d) show scanning electron microscope (SEM) images of the cross-section of (c) pure PU and (d) WPU.

FIG. 46 shows customized 3D-printed accessory to hold the device in a manner that avoids bending and fracture during the surgery according to certain embodiments of the invention, where (a) shows a schematic illustration of the accessory, and (b) shows an optical image of the accessory to hold the device on the cardiac surface.

FIG. 47 shows an equivalent circuit model for simultaneous wireless measurements according to certain embodiments of the invention, where (a) shows an equivalent circuit of the three LC-resonance sensing units in a multi-sensing element (top) and the wireless readout system (bottom), and (b) shows stretching the arms leads to a reduction of the capacitance and an increase in the resonance frequency.

FIG. 48 shows a flowchart for a custom Lab VIEW program for real-time data collection and analysis and subsequent further analysis according to certain embodiments of the invention.

FIGS. 49A and 49B show custom Lab VIEW program for real-time data collection and analysis and subsequent further analysis, where the program collects and analyzes the reflection coefficient (S₁₁) data measured with the ENA network analyzer in real time and converts the S₁₁ data to the real part of the impedance (Re(Z)) accordingly, with further calculations on the resonance frequency (fₛ) and Q factor (Q) in real time, albeit with low time resolution (maximum data frequency: ˜5 Hz; lower than the data collection frequency (˜25 Hz)).

FIG. 49C shows that the custom Lab VIEW program also post-processes all the data.

FIG. 50 shows coordinate transformation to convert the strains along 0°, 120°, and 240° for each arm to principal strains (εₓ and ε_{y}) and shear strain (γ_{xy}) according to certain embodiments of the invention.

FIG. 51 shows strain sensitivity and bending insensitivity of the multi-sensing element according to certain embodiments of the invention, where (a) shows functionality of the top cover. The top cover fixes the distance between the floating electrode and the pair of bottom electrodes; and therefore, the capacitance remains stable under curvature. (b) shows change in resonance frequency for each arm is ˜1.5-2% with a strain of 10%, suggesting a sensitivity of ˜200 kHz per unit strain, and (c) shows the change in resonance frequency is ˜3-4% with a strain of ˜18% and <0.2% under a bending angle of 90°, respectively, suggesting negligible sensitivity to bending.

FIG. 52 shows simulation results for the sensitivity to strain and bending according to certain embodiments of the invention, where (a) shows normalized capacitance exhibits a linear relationship with normalized overlap area/length (with fixed width) during stretching, and (b)-(c) show relationships between the capacitance change and the radius of curvature normalized by the device in-plane size L for the three capacitors as a result of x-and y-bending. The capacitance change under curvature is negligible compared with that under stretching.

FIG. 53A shows insignificant mechanical constraints on natural motions of the cardiac tissues according to certain embodiments of the invention, where (a) shows optical images from the two cameras for 3D-PTV, (b) shows 3D reconstruction of the cardiac tissue with device mounted, and (c)-(e) show strain distributions of the cardiac surface without the device, with the device, and with a rigid film in a similar structure (Young's modulus: ˜1 GPa) at maximum heart volume. FIG. 53B shows additional mechanical constraints of FIG. 53A, where (a)-(c) show quantitative assessments of the strain values (S1 and S2) along two lines in FIG. 53A(c)-(e) at full heart volume, and (d) shows strain values (S1) during the entire expansion-contraction process.

FIG. 54A shows results using a simplified model on the constraints to natural motions of cardiac tissues, where (1) shows a cross-sectional view of the device on the cardiac tissues, (2) shows a top view of the device on the cardiac tissues, (3) shows normalized strain (ε_{device}/εₕₑₐᵣₜ, which is the device strain ε_{device} divided by the heart strain εₕₑₐᵣₜ) as a function of heart strain, (4) shows the normalized strain as a function of heart modulus, and (5) shows the normalized strain as a function of heart thickness.

FIG. 54B shows results using a living heart model, where (1) shows strain distribution without the device and (2) shows strain distribution with the device at maximum heart expansion.

FIG. 55 shows ex vivo evaluations of the cardiac device on an artificial heart system with adjustable oscillatory flow and pressure, where (a) shows PA pressure and flow rate as references, (b) shows the resonance frequency data for each sensing unit, and (c) shows the maximum change in resonance frequency increases as the PA pressure rises in these three sensing units.

FIG. 56 shows principal and shear strains in ex vivo evaluations of the cardiac device, where (a) shows converted strain data along with each sensing unit, and (b) shows coordinate transformation of the strain values along the three arms determines the principal and shear strains.

FIG. 57 shows in vivo acute evaluations in an ovine model according to certain embodiments of the invention, where (a) shows a device designed for measurements on the LV of an ovine animal, (b) shows raw data from one sensing unit, (c) shows filtering the raw data yields the respiration rate (duration: ˜4-5 s; bandpass <1 Hz; marked red) and the heart rate (duration: ˜0.9 s; bandpass: 40-200 Hz; marked blue), (d)-(f) shows quantitative assessment of principal strains (d) εₓ and (e) ε_{y} and shear strain (f) γ_{xy} of the ovine LV.

FIG. 58 shows raw data for the three units in a representative segment (duration: 20 s).

FIG. 59 shows data filtering for raw data measured from the LV of the porcine heart according to certain embodiments of the invention, where data with a bandpass of <1 Hz determines the respiration rate, and data with a bandpass of 40-200 Hz determines the heart rate and the strains.

FIG. 60 shows quantitative assessment of the strains of the ovine LV along the three arms corresponding to FIG. 57(d)-(f).

FIG. 61 shows connection between the multi-sensing element and the flexible cable according to certain embodiments of the invention, where (a) shows bioresorbable conductive wax connects the ends of the six independent traces in the flexible cable to the six pads of the three pairs of bottom electrodes in the multi-sensing element, and (b) shows a bioresorbable polymer mixture based on PCL and shellac (ratio of PCL to shellac: 9:1) seals the connection sites, and the PCL provides encapsulation and the shellac provides adhesion.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this specification will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures. is turned over, elements described as being on the “lower” side of other elements would then be oriented on “upper” sides of the other elements. The exemplary term “lower”, can, therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this specification, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this specification, and will not be interpreted in an idealized or overly formal sense unless expressly so defined herein.

As used in this specification, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this specification, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in a different order (or concurrently) without altering the principles of the invention.

As discussed above, currently existing processes for constructing bioresorbable electronic platforms, which heavily rely on 1) microfabrication and transfer printing and 2) solution-based printing, remains tedious and complex. An ideal fabrication methodology must offer (1) compatibility with the full range of eco/bioresorbable materials, including conductors, semiconductors, dielectrics, and polymer substrates, (2) capabilities in precise dimensional control (thicknesses and lateral features) and accurate overlay registration, (3) straightforward routes to multi-layered, multi-material device architectures with functions to address various application scenarios.

In view of the aforementioned deficiencies, the inventors has considered using pulsed laser ablation methods, which exploit well-controlled light-matter interactions for surface patterning, with various demonstrated uses in manufacturing. The accuracy and precision of lateral patterning and thickness reduction depend not only on the properties of the materials but on key laser parameters, especially pulse duration and wavelength. Ultrashort pulsed lasers (less than 10 ps) are advantageous because the ablation process occurs on timescales sufficiently short to limit thermal diffusion and the associated spread of the heat-affected zone. Wavelengths ranging from ultraviolet (UV) to near infrared (NIR) for ultrashort pulsed lasers allow the ablation rates, as well as surface morphologies, to be tuned due to the wavelength-dependent reflectivity and absorption of the materials. The mechanism of ultrafast laser ablation involves explosive vaporization and melt cavitation. Direct sublimation to the gas phase and/or ionization into dense plasmas likely occur in these processes. Dedicated systems exist for manufacturing interconnect traces in flexible circuit boards and components of certain classes of thin-film sensors. These approaches are, however, most well developed to structures with modest resolution (lateral and vertical resolution of over 20 μm and 1 μm, respectively) and to single functional layers; and they have not been applied to semiconductor materials generally, nor to eco/bioresorbable materials specifically.

In certain embodiments, the inventors propose a high-speed, scanned, picosecond-pulsed laser ablation approach, which, combined with physical lamination and/or transfer printing, serves as a versatile method for fabricating advanced multi-layered eco/bioresorbable electronic systems through careful selection of laser parameters. One appeal of this scheme is in its alignment with current manufacturing and prototyping schemes for conventional flexible printed circuit boards (PCBs). When implemented in a repetitive fashion using materials delivered to a substrate in sequence by physical lamination, transfer printing or conventional deposition or growth techniques, this methodology can yield state-of-the-art eco/bioresorbable electronic components, including complementary metal-oxide-semiconductor (CMOS) devices, through several basic operational modes. Ablation removes and patterns nano/micromembranes of constituent materials in an efficient, controlled manner via picosecond-pulsed light-matter interactions, all in a fast, dry process with a minimal thermal load. The process can accurately control the thicknesses (to within ˜35 nm) and lateral dimensions (down to ˜5 μm by overlapping scans) of features formed using these materials, in multi-layered configurations with good overlay registration (2-3 μm). A collection of system-level demonstrations in sensors of pressure, physiological parameters, thermal characteristics, flow properties, biopotential signals, and mechanical strains across multiple organ systems in live animal models illustrate important applications. Specific examples include wireless, implantable monitors for physiological behaviors in rat models, thermal probes for microcapillary flow sensing in transplanted tissues/organs in porcine models, flexible multi-electrode arrays for biopotential sensing on the cerebral cortex in mouse models, and wireless systems for tracking principal and shear strains on the epicardial surfaces in ovine models. These collective results highlight the broad spectrum of possibilities enabled by this fabrication technology.

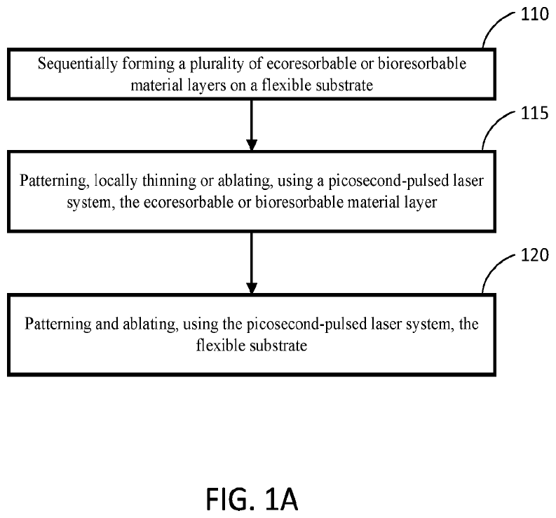

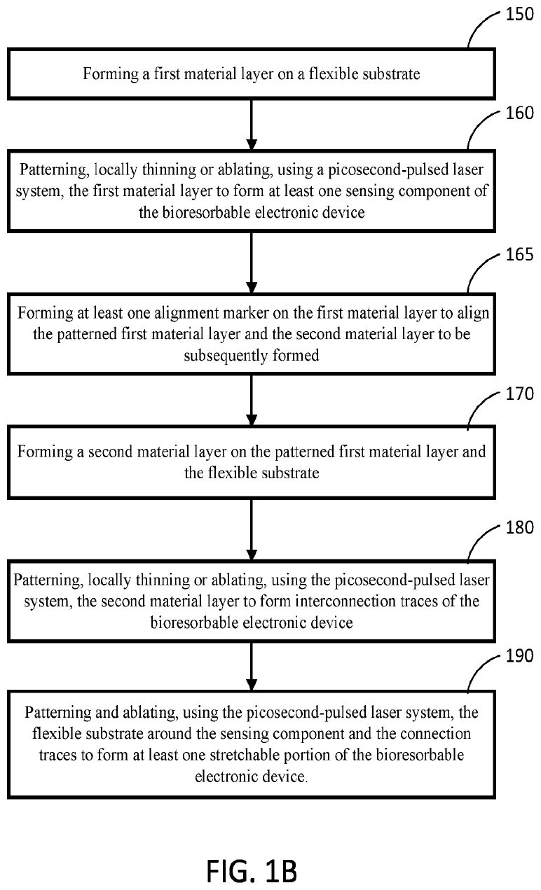

Aspects of the invention relate to methods for performing laser structuring of multi-layered ecoresorbable or bioresorbable materials and fabricating a bioresorbable electronic device using pisosecond-pulsed laser, devices fabricated by the methods, and applications of the same. For example, FIG. 1A shows a flowchart of a method for performing laser structuring of multi-layered ecoresorbable or bioresorbable materials according to certain embodiments of the invention. It should be particularly noted that, unless otherwise stated herein, the steps of the method may be arranged in a different sequential order, and are thus not limited to the sequential order as shown in FIG. 1A.

As shown in FIG. 1A, at procedure 110, a plurality of ecoresorbable or bioresorbable material layers are sequentially formed on a flexible substrate. In certain embodiments, the material layers are formed on the flexible substrate by physical lamination, transfer printing, deposition or growth techniques. At procedure 115, using a picosecond-pulsed laser system, the ecoresorbable or bioresorbable material layers are performed with patterning, locally thinning or ablating. The use of the picosecond-pulsed laser is advantageous because the ablation process occurs on timescales sufficiently short to limit thermal diffusion and the associated spread of the heat-affected zone. In certain embodiments, the patterning, locally thinning or ablating of the ecoresorbable or bioresorbable material layers are used to form at least one sensing component and interconnection traces of a bioresorbable electronic device. Then, at procedure 120, using the picosecond-pulsed laser system, the flexible substrate may be performed with patterning and ablating, thus forming the stretchable portions of the bioresorbable electronic device. In certain embodiments, a patterned structure of the ecoresorbable or bioresorbable material layers has a resolution of about 5-10 μm and alignment accuracy of less than 5 μm.

In certain embodiments, the laser structuring of multi-layered ecoresorbable or bioresorbable materials may be utilized for fabricating a multi-layered bioresorbable electronic device. For example, FIG. 1B shows a flowchart of a method for fabricating a multi-layered bioresorbable electronic device according to certain embodiments of the invention. It should be particularly noted that, unless otherwise stated herein, the steps of the method may be arranged in a different sequential order, and are thus not limited to the sequential order as shown in FIG. 1B.

As shown in FIG. 1B, at procedure 150, a first material layer is formed on a flexible substrate. The first material layer is ecoresorbable or bioresorbable. For example, the first material layer may be a semiconductor layer. In certain embodiments, the first material layer may be formed on the flexible substrate by physical lamination, transfer printing, deposition or growth techniques. Then, at procedure 160, using a picosecond-pulsed laser system, the first material layer is performed with patterning, locally thinning or ablating to form at least one sensing component of the bioresorbable electronic device. After patterning, locally thinning or ablating the first material layer, at procedure 165, at least one alignment marker is formed on the first material layer to align the patterned first material layer and a second material layer to be subsequently formed. Then, at procedure 170, the second material layer is formed on the flexible substrate and the patterned first material layer. The second material layer is also ecoresorbable or bioresorbable. For example, the second material layer may be a metal layer. In certain embodiments, the second material layer may be formed on the flexible substrate by physical lamination, transfer printing, deposition or growth techniques. Then, at procedure 180, using the picosecond-pulsed laser system, the second material layer is performed with patterning, locally thinning or ablating to form interconnection traces of the bioresorbable electronic device. Finally, at procedure 190, using the picosecond-pulsed laser system, the flexible substrate may be performed with patterning and ablating around the at least one sensing component and the connection traces to form stretchable portions of the bioresorbable electronic device.

In certain embodiments, the picosecond-pulsed laser system may be operated with a certain wavelength, a certain pulse duration, and a certain beam diameter. For example, in one embodiment, the picosecond-pulsed laser system is operated with a wavelength of about 1030 nm, a pulse duration of about 1.0 picoseconds, and a beam diameter of 15 μm.

In certain embodiments, the flexible substrate may be a flexible biodegradable polymeric substrate. In one embodiment, the flexible substrate is formed by polylactic acid (PLA) or cellulose acetate (CA). In certain embodiments, the bioresorbable electronic device has a resolution of about 5-10 μm and alignment accuracy of less than 5 μm.

### Laser Ablation Approaches for Eco/Bioresorbable Electronics

The picosecond-pulsed laser ablation approaches described in the embodiments enable high-resolution (1) patterning of films through the removal of material from defined regions, (2) thinning of films through reductions in thickness in patterned geometries, with minimal damage to underlying layers, and (3) cutting through films and their supporting substrates. A broad range of well-established bioresorbable materials, ranging from films, membranes and/or foils conductors, semiconductors, dielectrics to encapsulants and substrates prepared through lamination, transfer printing and/or deposition/growth, can be processed using these schemes. Repetitive application of preparation-ablation cycles can yield multi-layered structures for advanced electronic designs. This scheme offers a simple alternative to conventional cleanroom fabrication methods and photolithography techniques, and it avoids the need for any wet processing steps, including those that use aqueous solutions for etching, developing, cleaning, or washing, thereby eliminating any potential for degradation of the eco/bioresorbable materials, most of which are water-soluble.

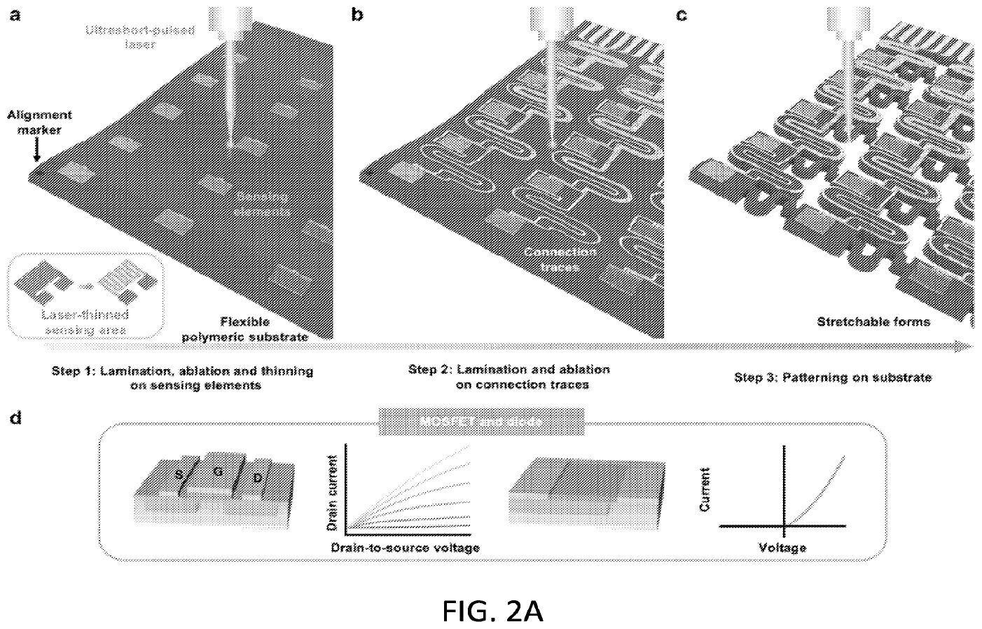

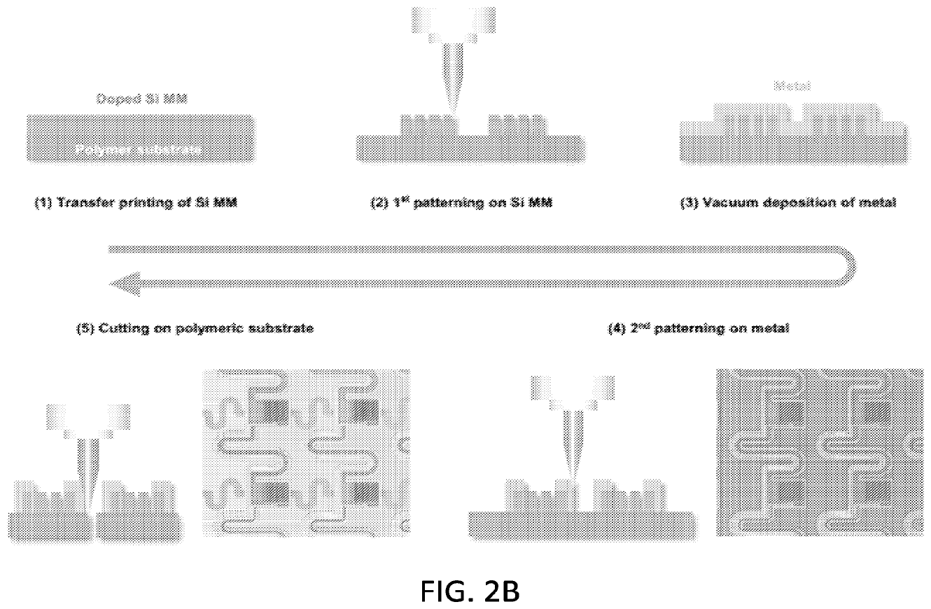

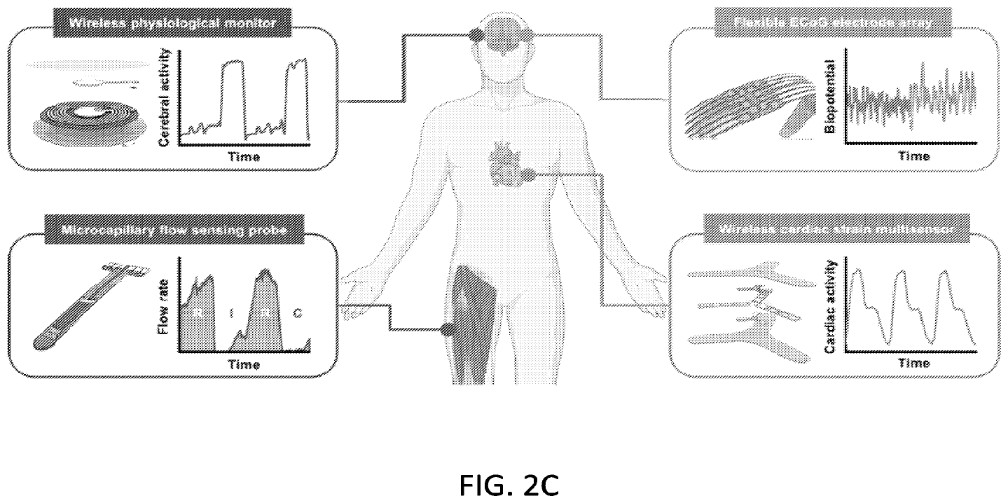

FIG. 2A schematically shows (a)-(c) an exemplary process of fabricating a bioresorbable electronic device using ultrashort-pulsed laser according to certain embodiments of the invention; and (d) representative electronic devices according to certain embodiments of the invention. FIG. 2B schematically shows cross-sectional views of laser ablation procedures for a bioresorbable electronic device using ultrashort-pulsed laser according to certain embodiments of the invention. Specifically, as shown in FIG. 2A, the process includes: (a) patterning of sensing elements and structuring of alignment markers, (b) patterning of the interconnection traces, and (c) structuring the structure into stretchable forms between the sensing elements and the interconnection traces. In this way, the representative bioresorbable electronic devices may be directly formed on the flexible biodegradable polymer substrate. FIG. 2B shows an example of the procedures for forming a multi-layered bioresorbable device that consists of an array of functional elements on a poly (lactic acid) (PLA) substrate, where monocrystalline Si micromembranes (Si MMs) serve as sensing elements and patterned Mg films act as electrical interconnections. In the first cycle, the process defines cuts (diameter: 1.5 mm) through the substrate as alignment marks for subsequent cycles of patterning of various material structures from uniform films or foils. The final step cuts the substrate into a serpentine structure to provide some effective level of stretchability, according to the principles of soft electronics.

FIG. 2C schematically shows examples of bioresorbable electronic devices being fabricated according to certain embodiments of the invention. As shown in FIG. 2A(d) and FIG. 2B, the different representative device types include, without being limited thereto, Si-based semiconductor devices such as a MOSFET and a diode. System examples include wireless monitors for physiological parameters in tissue cavities, probes for microcapillary flow sensing in transplanted tissues/organs, flexible Si-based electrode arrays for biopotential recordings, and wireless systems for measuring principal and shear strains on the epicardial surface.

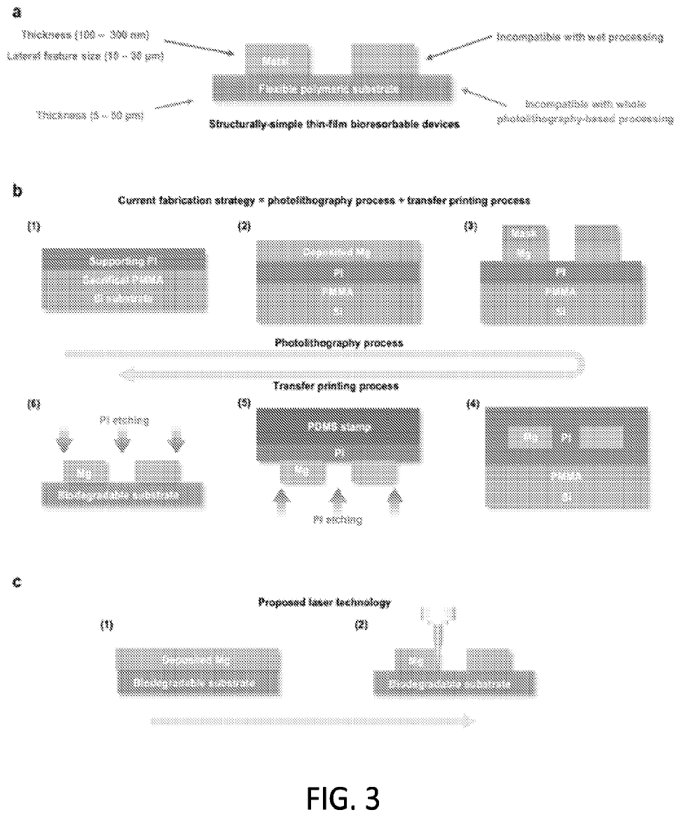

FIG. 3 schematically shows a comparison of the current fabrication methods of a bioresorbable electronic device and the method for fabricating bioresorbable electronic device using ultrashort-pulsed laser according to certain embodiments of the invention. As shown in FIG. 3(a), Structurally-simple thin-film bioresorbable devices include a top functional metal layer (thickness: 100-300 nm; lateral feature size: 5-30 μm) and a flexible polymer substrate (thickness: 5-50 μm). A challenge is that the metal is incompatible with wet processing, and the bottom polymer substrate is incompatible with photolithography-based processing. As shown in FIG. 3(b), the current fabrication scheme includes photolithography-based microfabrication and transfer printing of the patterned devices. The specific procedures consist of (1) spin-coating layers of supporting PI and sacrificial PMMA on a Si substrate, (2) vacuum deposition of a uniform layer of Mg, (3) patterning of a mask layer by photoresist (e.g., Fe), patterning of the Mg layer using diluted HCl, and removing the mask layer, (4) spin-coating another supporting PI layer, (5) releasing the sacrificial PMMA, retrieving the PI-Mg-PI tri-layer structure, and performing RIE on the bottom PI, and (6) transferring to the bioresorbable polymer substrate and performing RIE on the top PI. In comparison, as shown in FIG. 3(c), The proposed laser fabrication scheme introduced here includes (1) vacuum deposition of a uniform layer of Mg directly on a bioresorbable polymer substrate and (2) laser ablation of the Mg layer into desired patterns.

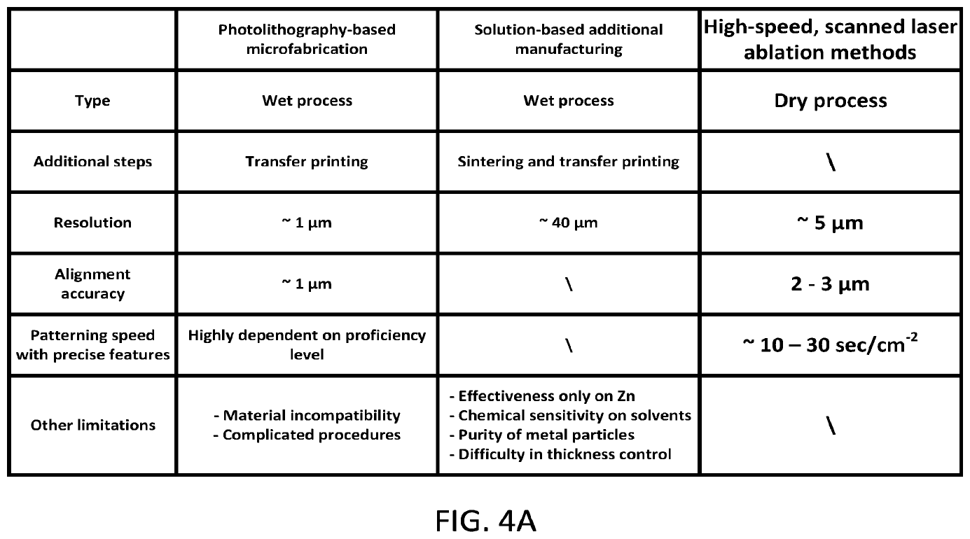

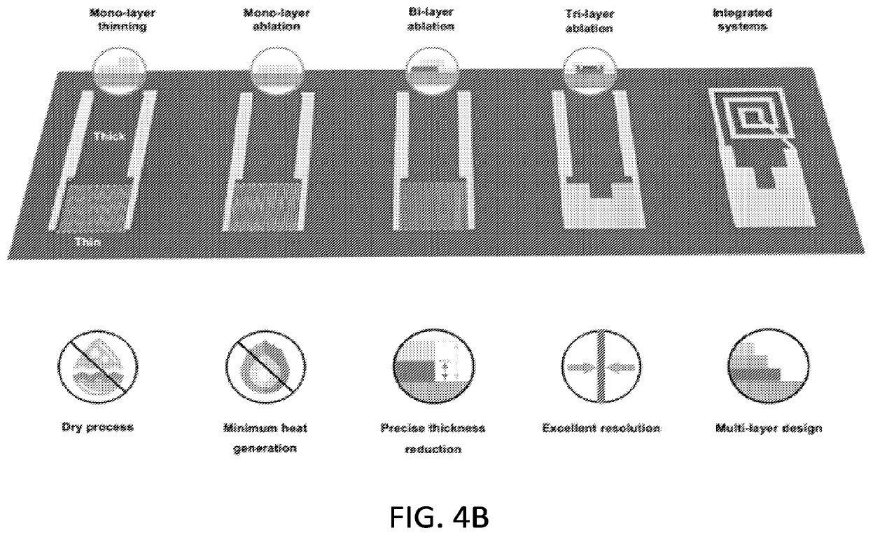

FIG. 4A shows a table of comparison between the method for fabricating a bioresorbable electronic device using ultrashort-pulsed laser according to certain embodiments of the invention and existing technologies. FIG. 4B schematically shows representative laser-processed bioresorbable devices according to certain embodiments of the invention. As shown in FIG. 4B, the representative laser-processed bioresorbable device types include mono-layer thinned and ablated devices, multi-layer ablated devices, and integrated systems, with features including dry processing, minimum heat generation, precise thickness reduction, excellent resolution, and multi-layer design. Specifically, FIGS. 4A and 4B summarize key features of the method is processing sequence. Firstly, in the method utilized in certain embodiments of the invention, the procedures are entirely dry and they involve minimum heat generation resulting from ultrashort light-matter interactions associated with the use of a picosecond-pulsed laser. These characteristics minimize thermally induced degradation or damage to the constituent materials. Secondly, by careful selection of the parameters for ablation, material removal can occur in a well-controlled manner, with high lateral resolution (5 μm) and good overlay registration (2-3 μm). Thirdly, complex single and multi-layered device structures are possible, including those that incorporate device-grade monocrystalline silicon, such as metal-oxide-semiconductor field-effect transistors (MOSFETs) and diodes. A collection of systems for measuring pressure, physiological parameters, thermal characteristics, flow properties, biopotential signals, and mechanical strains across various organ systems appears in FIG. 2A(d). Specific embodiments range from wireless monitors for physiological behaviors in tissue cavities, probes for microcapillary flow sensing in transplanted tissues/organs, flexible Si-based electrode arrays for biopotential sensing on tissue surfaces, and wireless systems for tracking principal and shear strains on the epicardium.

### Characterization of the Ablation Process

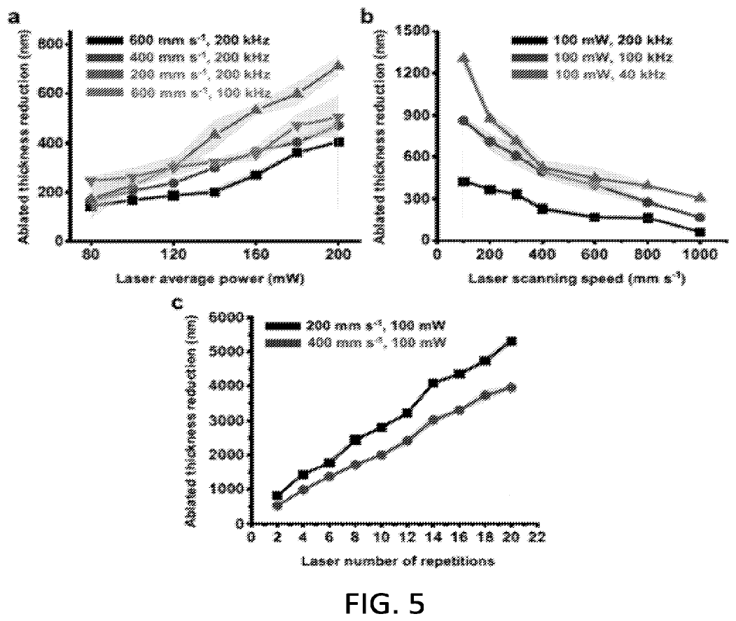

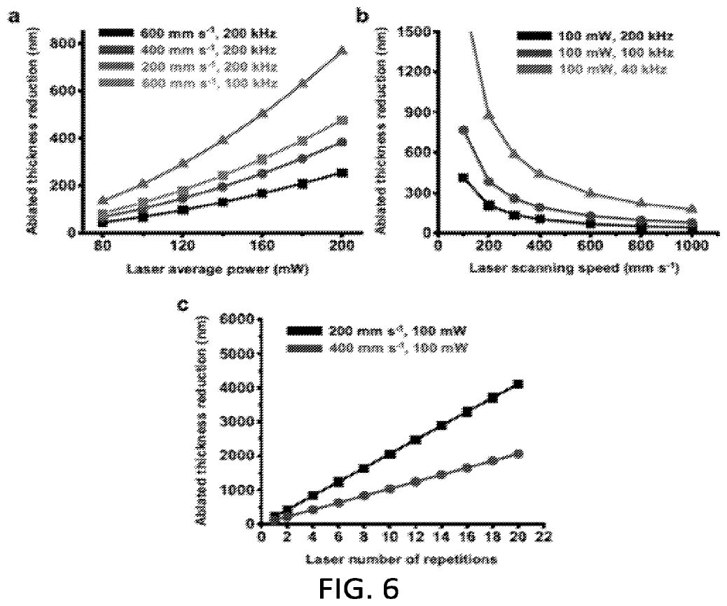

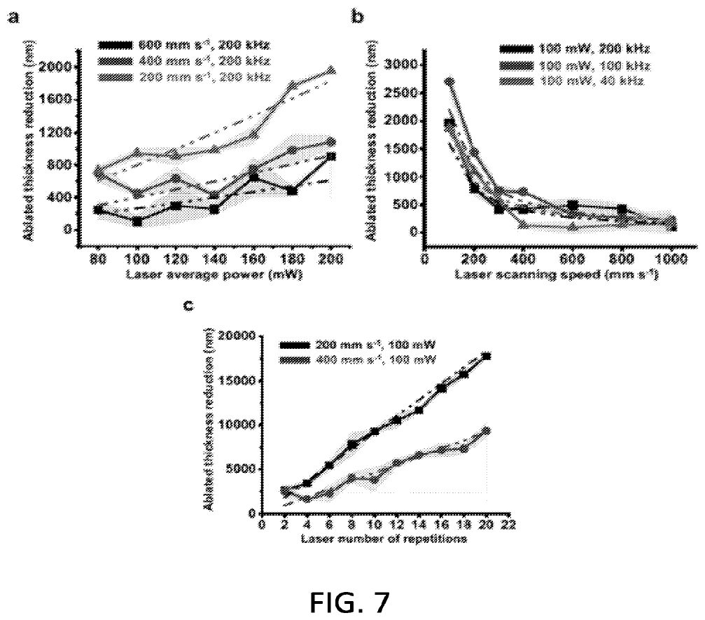

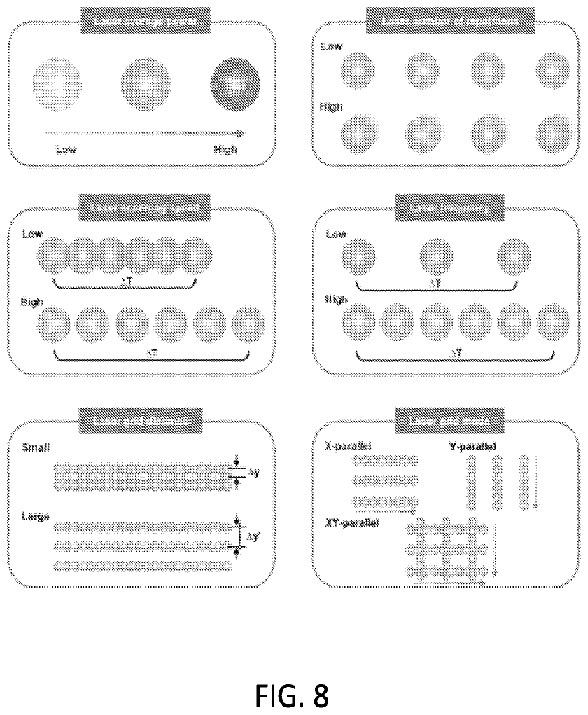

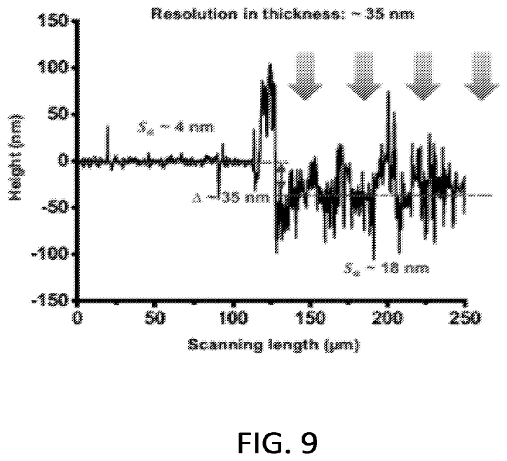

FIG. 5 shows controlled reductions in thicknesses of monocrystalline Si MMs achieved by ablation, and their dependence on ablation parameters, including (a) average power, (b) scanning speed and frequency, and (c) number of repetitions at fixed grid distance (7 μm) and grid mode (XY-parallel) according to certain embodiments of the invention. FIG. 6 shows simulation results for controlled thickness reduction monocrystalline Si MM by tuning the laser parameters, and their dependence on ablation parameters, including (a) average power, (b) scanning speed and frequency, and (c) number of repetitions according to certain embodiments of the invention. FIG. 7 shows experimental and simulation results for controlled thickness reduction in Mg by tuning the laser parameters, and their dependence on ablation parameters, including (a) average power, (b) scanning speed and frequency, and (c) number of repetitions according to certain embodiments of the invention. FIG. 8 schematically shows illustrations of key parameters used for ablation using ultrashort pulsed lasers according to certain embodiments of the invention. Specifically, FIGS. 5-7 present examples of patterned reductions in the thicknesses of micromembranes of device-grade silicon and thin films of Mg through control of key laser parameters such as average power (P; 80-200 mW; FIG. 5(a)), scanning speed (v; 200-600 mm s⁻¹; FIG. 5(b)), frequency (f, 40-200 kHz; FIG. 5(b)), and the number of repetitions (n; 2-20; FIG. 5(c)) experimentally and theoretically, at fixed grid distance (d, 7 μm) and grid mode (XY-parallel) as described in detail in FIG. 8. The thickness reduction increases monotonically with average power and number of repeats and decreases with scanning speed and frequency. The peak power increases while the frequency decreases when the average power remains unchanged. A simple, empirical model assumes that the intensity of the focused laser beam follows a Gaussian profile with a diameter d₀ and that the maximum thickness reduction by a single laser pulse (Dₘₐₓ) occurs at the circular central region, increasing with the pulse energy W, where W=P/f, and with a magnitude that depends on material properties such as the heat capacity, density, vaporization temperature, and heat of vaporization and on the laser parameters such as the wavelength, the pulse duration, the peak intensity, and others. An empirical formula fitted from the experimental results gives Dₘₐₓ=α×W^{β}, where α and β depend on material properties (e.g., α=1×10⁵ nm mJ^{-1/β} and β=1.9 for monocrystalline Si; α=4×10³ nm mJ^{-1/β} and β=1.2 for Mg). Descriptions of the nature of these nonlinearities can be found elsewhere. The ablated thickness decreases with the distance to the center of the circular region (r) according to a Gaussian function D=Dₘₐₓexp(−kr²d₀⁻²), where k is an empirical parameter that defines the thickness profile, and is found to take a value of ˜50 for the experimental results presented here. The total thickness ablated by multiple pulses is a summation of that by an individual pulse. The experimental results agree well with those based on theoretical models of the ablation process. The minimum reduction in thickness is ˜35 nm for the laser system and the Si MMs examined here (see FIG. 9). Specific parameters depend on material properties, as expected.

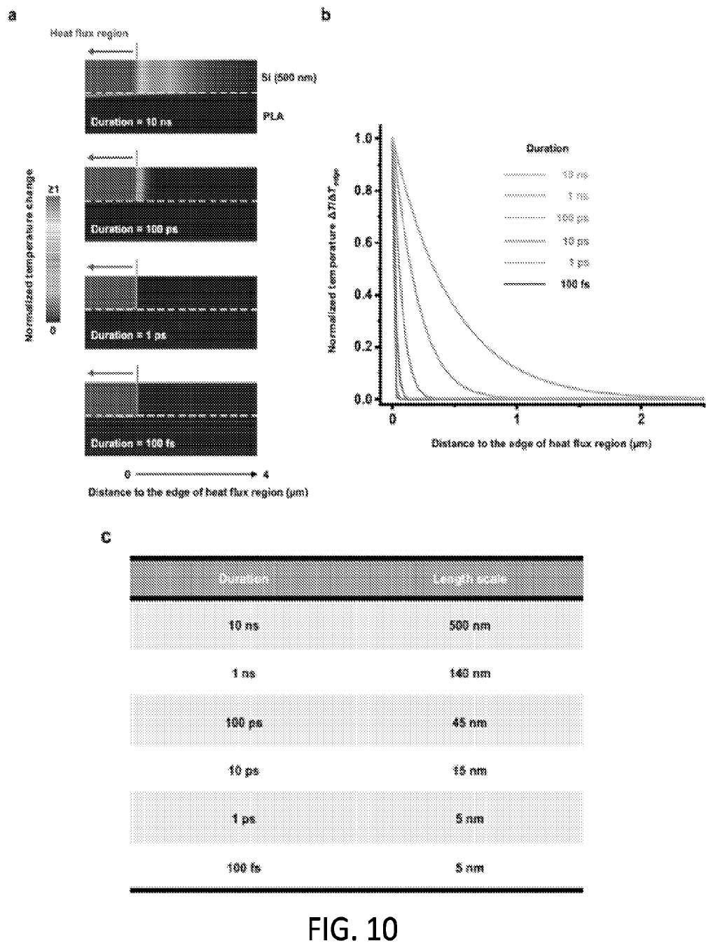

Additionally, numerical simulation in FIG. 10 reveals that the length scale for a body heat flux with the duration used in our system (˜1 ps) is ˜5 nm, which is negligible compared to the lateral patterning resolution (˜5 μm). FIG. 10 shows simulations of the thermal diffusion zone induced by a body heat flux with durations t from 10 ns to 100 fs. Specifically, FIG. 10(a) shows normalized temperature distribution on a Si/PLA (thickness: 500 nm/50 μm) structure at time t. The temperature values are normalized by the temperature at the edge of the heat flux region. The results from top to bottom are the thermal diffusion zones associated with durations of 10 ns, 100 ps, 1 ps, and 100 fs, respectively. FIG. 10(b) shows normalized temperature as a function of the distance to the edge of the heat flux region for different durations. Durations that are less than 1 ps minimize heat transfer to the surrounding areas. FIG. 10(c) shows length scale values with different durations, which highlights that the ultrashort pulsed laser efficiently minimizes the thermal diffusion zone adjacent to the edges of patterned features. Specifically, length scale values with different durations appear in FIG. 10(c). The results highlight that the ultrashort pulsed laser efficiently minimizes the thermal diffusion zone adjacent to the edges of patterned features.

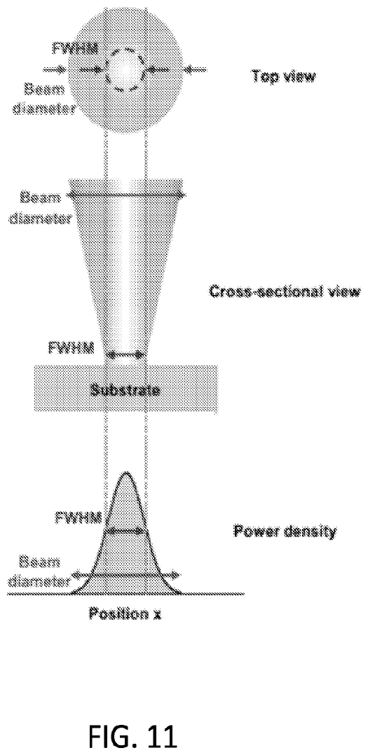

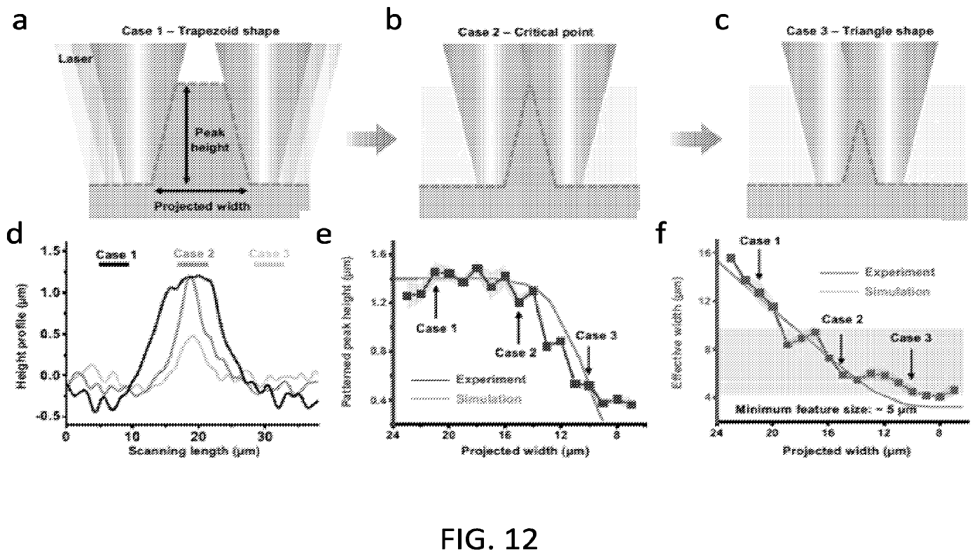

FIG. 11 schematically shows different views of Gaussian distribution of the power density along the radial direction of the laser spot (diameter: ˜15 μm) according to certain embodiments of the invention. As shown in FIG. 11, the lateral resolution of ˜5 μm follows from the use of a 15-μm-diameter, Gaussian laser spot. FIG. 12 shows schematic views of cross-sectional profiles of ribbon-shaped structures of monocrystalline Si MM formed by ablation in different cases, where (a) shows a trapezoid shape with a titled edge (projected width larger than the laser spot diameter Dₗₐₛₑᵣ in case 1, (b) shows a triangle shape at the critical point (projected width similar to Dₗₐₛₑᵣ in case 2, and (c) shows a proportionally reduced triangle shape (projected width smaller than Dₗₐₛₑᵣ in case 3; and (d) shows experimentally measured profiles for these three cases.

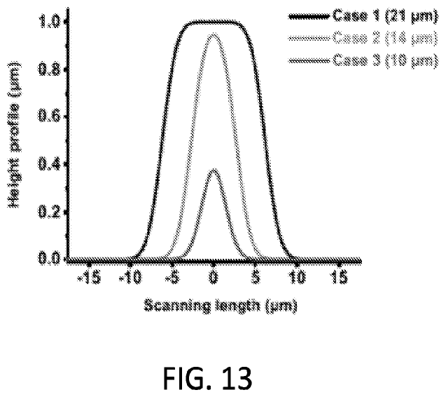

Overlapping scans provide additional capabilities in resolution, as illustrated in the case of two sequential scans at various separations, referred to as projected widths, as in FIG. 12(a)-(c). The Si ribbons that result from this process possess trapezoidal cross-sectional dimensions in case 1 in FIG. 12 (a) with projected widths larger than the laser spot diameter. Reducing the projected width to values comparable to this diameter in case 2 in FIG. 12(b) yields a triangular cross-section. Further reductions decrease the size of the triangle proportionally, leading to a reduced cross-sectional area in case 3 in FIG. 12(c). FIG. 12(d) summarizes experimentally measured cross-sectional profiles for these three scenarios, consistent with simulation results (see FIG. 13).

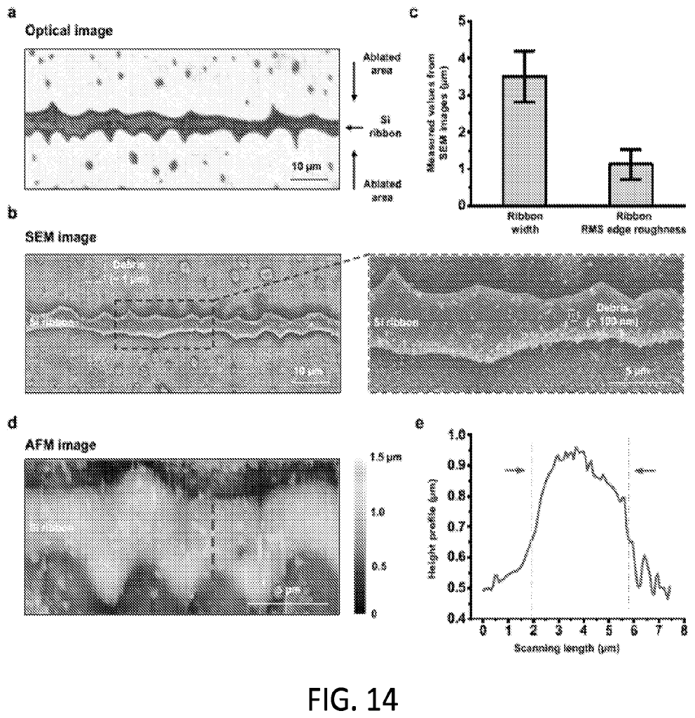

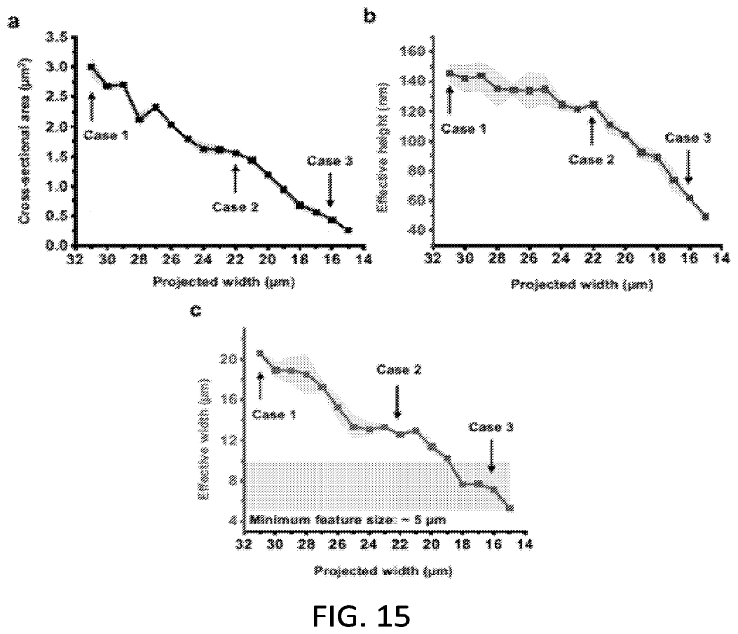

FIG. 12(e) and (f) present the peak heights and effective widths (integrated cross-sectional area divided by the peak height) of the patterned Si ribbons, respectively, as a function of projected width determined experimentally and theoretically. The effective width corresponds to the integrated cross-sectional area divided by the peak height. The minimum feature size is ˜5 μm. The shaded areas denote the standard deviation. As shown in FIG. 12(e), the peak height values in cases 1 and 2 are similar. However, the values decrease in case 3 with the reduction in the projected width. As shown in FIG. 12(f), the effective width, which defines the width of the ribbons, decreases continuously with the projected width, to a minimum value of ˜5 μm in case 3. Optical, scanning electron microscope (SEM), and atomic force microscope (AFM) images from the narrowest monocrystalline Si ribbons (width: ˜4-5 μm) on a PLA substrate appear in FIG. 14. Similar scenarios and minimum feature sizes occur in patterning trench shapes, as shown in FIG. 15, which shows quantitative characterization of a trench shape formed by laser method, where (a) shows cross-sectional area, (b) shows peak height, and (c) shows effective width as functions of projected width. The minimum feature size is ˜5 μm. The shaded areas denote the standard deviation. These and other important capabilities of the process rely on accurate registration through multiple cycles of patterning.

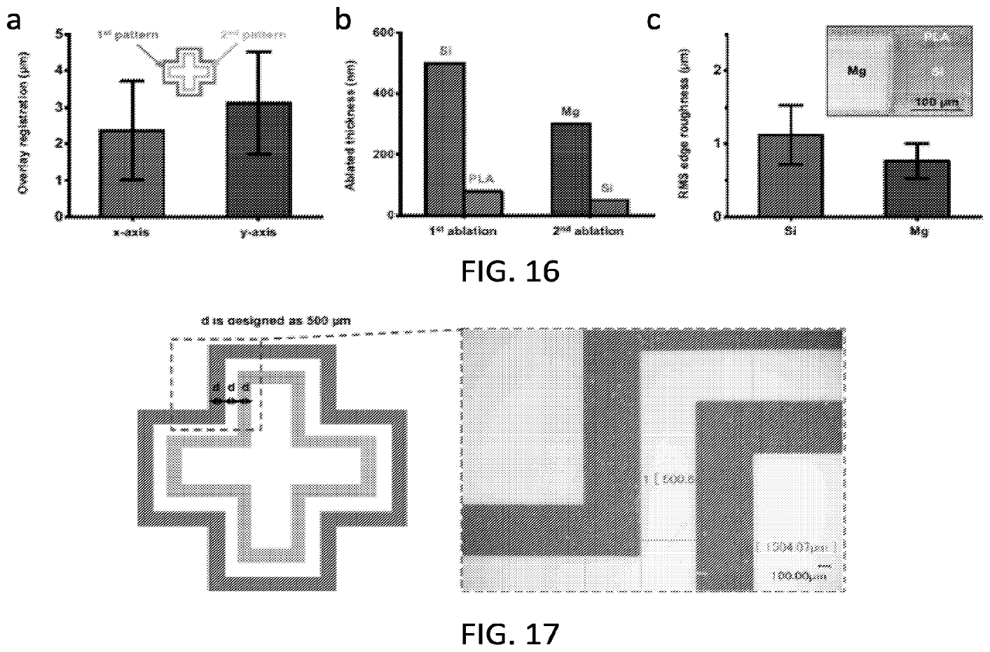

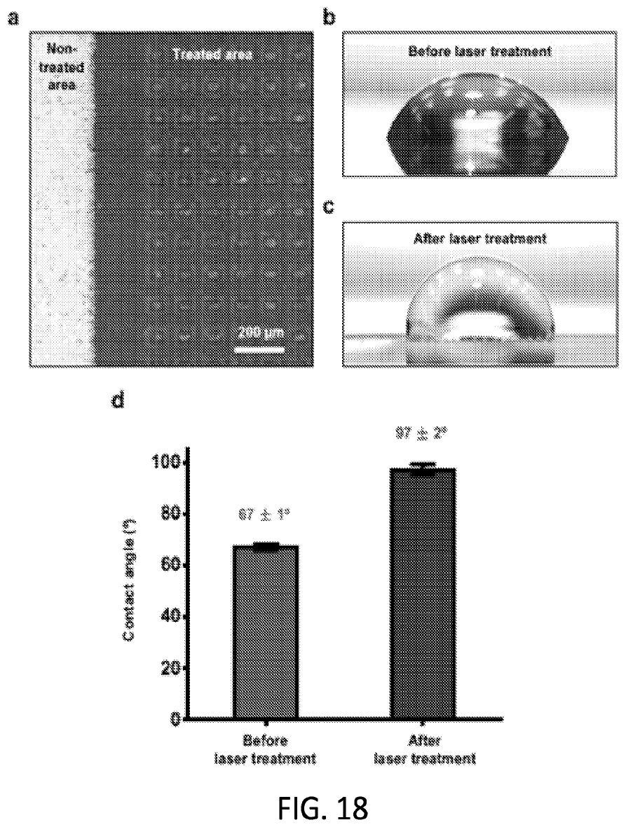

FIG. 16 shows (a) alignment accuracy in x-and y-axis is 2.7±1.3 and 3.1±1.2 μm, respectively, (b) minimized damage to underlying materials following laser ablation processing of the top layer, ablating a 500-nm-thick top Si MM layer leads to an ablated thickness of ˜80 nm for the underlying PLA layer (original thickness: 50 μm); ablating a 300-nm-thick top Mg layer leads to an ablated thickness of ˜50 nm for the underlying Si layer (original thickness: 500 nm), and (c) root-mean-square (RMS) line edge roughness for Si (thickness: 2 μm; width: 100 μm) and Mg (thickness: 500 nm; width: 100 μm) ribbons patterned on PLA substrates (thickness: 50 μm) are 1.1+0.4 μm and 0.8+0.2 μm, respectively, and insets showing SEM images (titled angle: 45°) of a bi-layer Mg/Si structure (thickness: 300 nm for top Mg and 2 μm for bottom Si) on a PLA substrate (thickness: 50 μm). FIG. 17 shows a schematic view and an optical micrograph of the pattern for characterizing overlay registration, corresponding to FIG. 16(a). The distance between two crisscross patterns is designed to 500 μm. The difference between the actual measured distance and the designed distance determines the overlay registration. The results in FIG. 16(a) and FIG. 17 indicate that the overlay registration along the x-axis and y-axis is 2.7±1.3 and 3.1±1.2 μm, respectively.

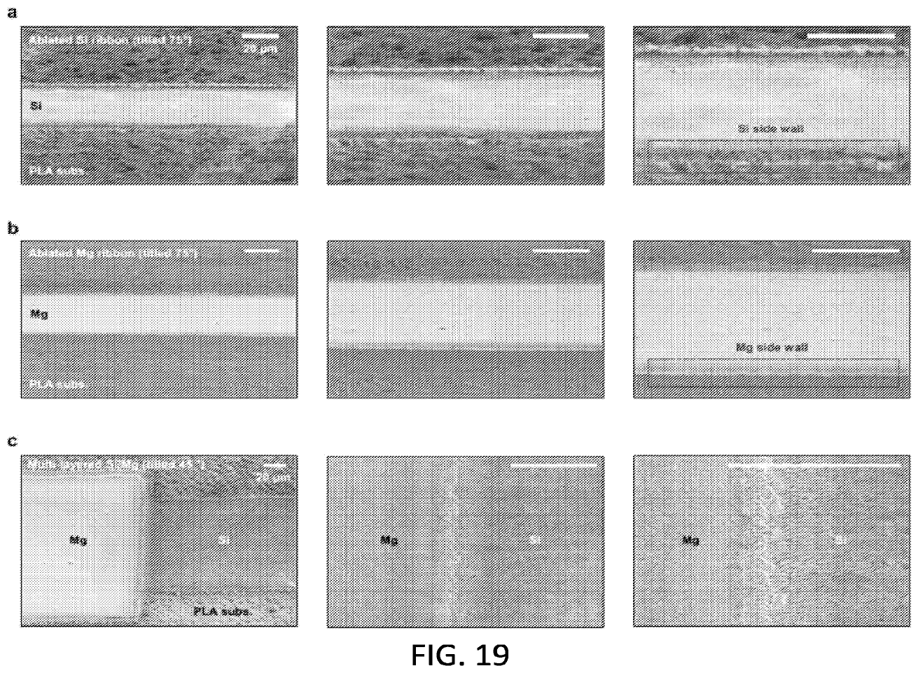

This controlled ablation process extends to the ability to pattern or to reduce the thicknesses of surface layers with minimum damage to the underlying materials. Two examples illustrate this capability—complete removal of a top layer of Si (thickness: 500 nm) on PLA (initial thickness: 50 μm) and of Mg (thickness: 300 nm) on Si (initial thickness: 500 nm), as summarized in FIG. 16(b). Characterization by 3D laser scanning microscopy indicates that the depth of damage to the substrate in both cases (depth for PLA: ˜80 nm; depth for Si: ˜50 nm) is small compared with the initial thicknesses of the overlying layers. FIG. 18 shows the laser ablation method for surface treatment on substrates, where (a) shows laser-treated micro-textured PLA surface (right) compared with pristine PLA surface (left), (b) and (c) show cross-sectional images of a water droplet (volume: 1.5 μL) on (b) the pristine PLA surface (c) laser-treated micro-textured PLA surface, and (d) shows contact angle values of the pristine and laser-treated surfaces are 67±1° and 97±2°, respectively. The laser treatment enhances surface hydrophobicity. Related surface-selective processing can be used to form micro-textures that define the morphology and control related properties such as wettability, as illustrated in FIG. 18. The line edge roughness of patterned ribbons of Si (thickness: 2 μm; width: 100 μm) and Mg (thickness: 500 nm; width: 100 μm) on a PLA substrate (thickness: 50 μm) appears in FIG. 16(c). The root-mean-square (RMS) values are 1.1±0.4 μm for Si and 0.8±0.2 μm for Mg, respectively.

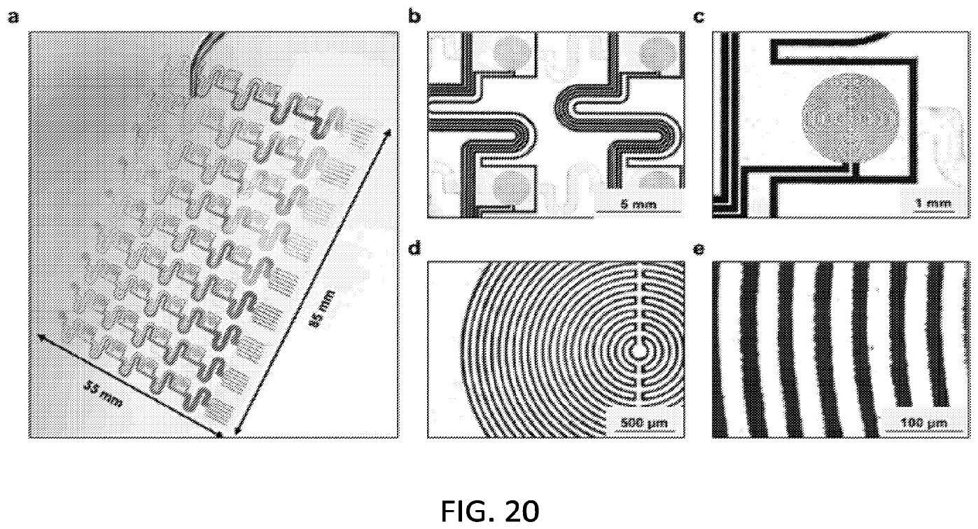

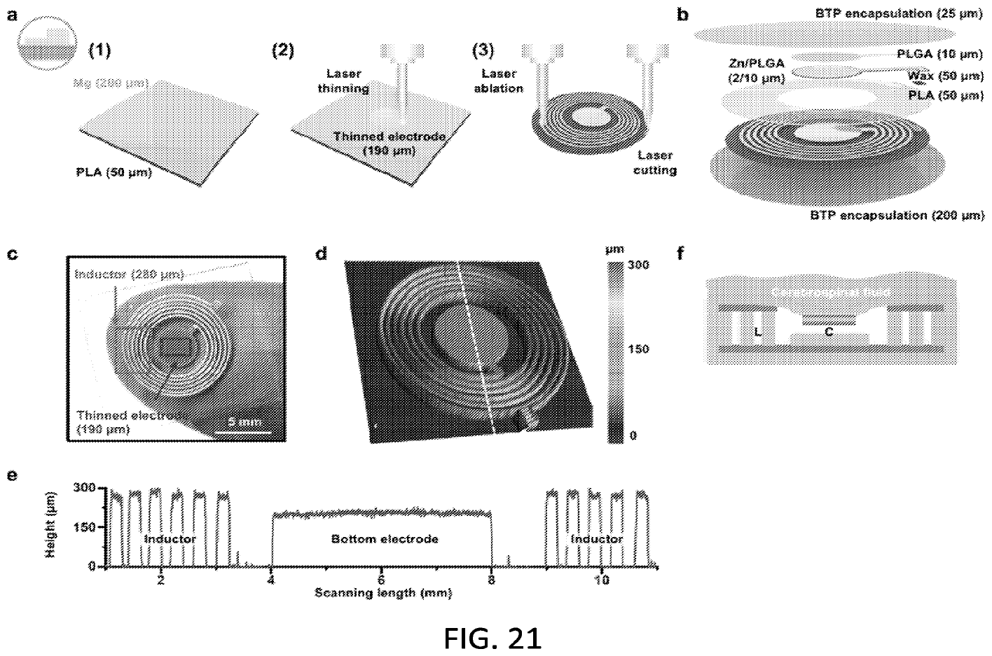

FIG. 19 shows SEM characterization of laser-ablated Si and Mg structures, where (a) shows SEM images (tilted angle: 75°) of an ablated Si ribbon (width: 50 μm; thickness: 2 μm) on a PLA substrate (thickness: 50 μm), (b) shows SEM images (tilted angle: 75°) of an ablated Mg ribbon (width: 50 μm; thickness: 500 nm) on a PLA substrate (thickness: 50 μm), and (c) shows SEM images (titled angle: 45°) of a bi-layer Mg/Si structure (thickness: 300 nm for top Mg and 2 μm for bottom Si) on a PLA substrate (thickness: 50 μm). Top and side views of the surface morphologies of Si and Mg ribbons and bi-layer Si/Mg structures appear in SEM images in FIG. 19 and inset of FIG. 16 (c). The process also allows the formation of large-area arrays. FIG. 20 shows capability for forming arrays of devices over large areas, including optical micrographs of an array (materials: Mg/PLA; thickness: 0.3/50 μm; dimension: 85×55 mm) of 45 thin-film resistive-type temperature sensors (arrangement: 9×5), on a serpentine-type substrate of PLA, to allow stretchability. Specifically, FIG. 20 shows an array of thermistors (materials: Mg/PLA; thickness: 0.3/50 μm) serves as a representative example, which illustrates large-area fabrication (dimension: 85×55 mm) of many (45 in a 9×5 configuration for this example) devices on a PLA substrate patterned into a stretchable, serpentine mesh. Related ablation processes are used at the industrial scale, with patterning speeds that depend on the laser scanning speed, the patterns of these scans, and the number of repetitions. For monocrystalline Si, as an example of interest to the present work, the patterning speed is ˜10-30 sec cm⁻² with the laser system used here when the scanning speed, grid distance, and the number of repetitions are 1000 mm s⁻¹, 7 μm, and 1, respectively.

### Processing by Thickness Reduction for Wireless Bioresorbable Physiological Monitors

Tracking internal pressures and other physiological parameters forms the basis of many operations in patient monitoring, often needed only for periods of time that coincide with a temporary condition such as recovery from a surgical operation. An exemplary bioresorbable device that can be useful in this context includes a circular inductor with a thinned bottom electrode (material: Mg; thickness: 280 μm for coil and ˜190 μm for bottom electrode; substrate: 50-μm-thick PLA), an annular spacer (material: PLA; thickness: 50 μm), a top electrode (material: PLGA/Zn/PLGA; thickness: 10/2/10 μm), and top and bottom layers of bioresorbable thermoplastic polymer (BTP; synthesis process in methods section) for encapsulation (thickness: 25 μm for top and 200 μm for bottom).

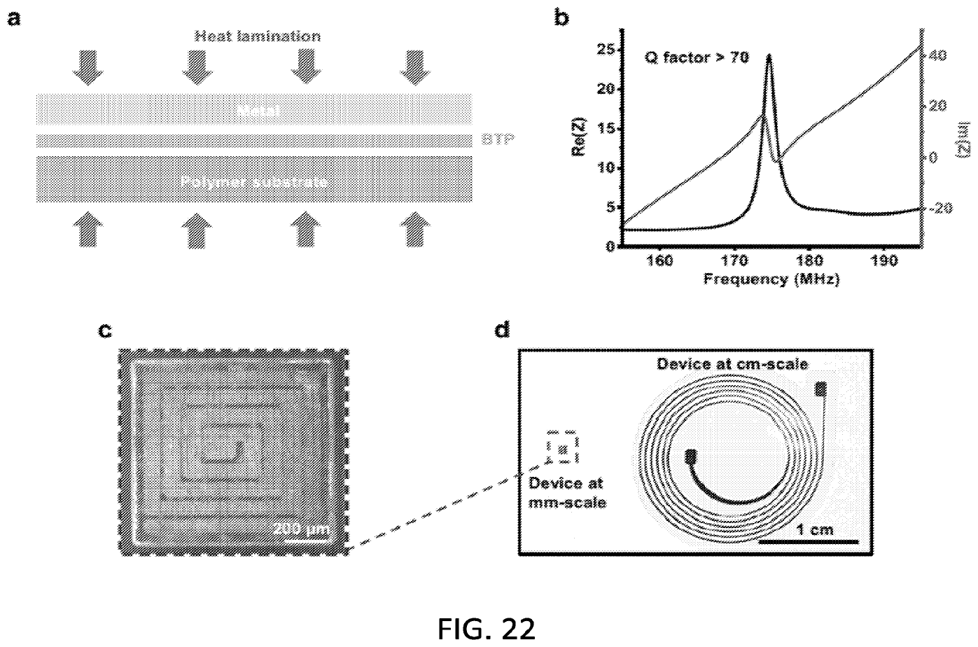

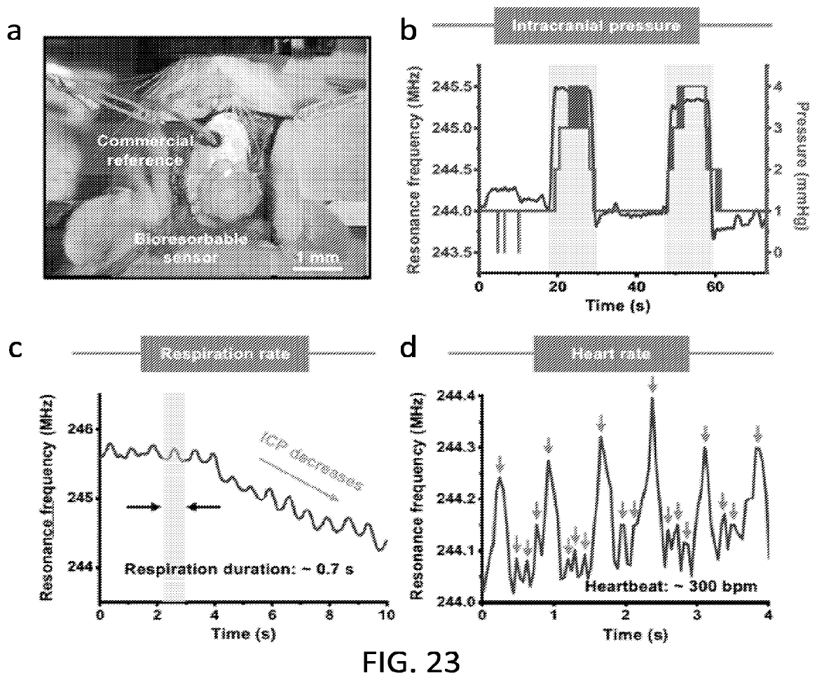

FIG. 21 shows the strategy for thinning material structures used in wireless, bioresorbable physiological monitors according to certain embodiments of the invention, and FIG. 22 shows laser ablation strategy for bioresorbable inductors at the cm-and sub-mm scales according to certain embodiments of the invention. Specifically, FIG. 21(a) shows the process applied to an inductor with a thinned bottom electrode, as a key component in such a device. As shown in FIG. 21(a), the procedures include (1) lamination of a uniform Mg layer (thickness: 280 μm) on a PLA substrate (thickness: 50 μm) using the BTP interfacial adhesive, (2) thinning of the Mg layer by ablation to reduce the thickness of the central circular area (diameter: 4 mm) from 280 to 190 μm for the bottom electrode, and (3) ablation of the surrounding Mg to define a helical coil structure and to cut the PLA substrate into the outline of the device. The procedures for the inductor as shown in FIG. 21(a) incorporates a central thinned bottom electrode as a critical component of the device. Bonding a Mg foil (thickness: 280 μm) on a PLA substrate (thickness: 50 μm) with BTP as an adhesive, as shown in FIG. 22(a) completes the preparation of the material stack, as shown in step (1) of FIG. 21(a). The ablation process reduces the thickness of the central circular area (diameter: 4 mm) from 280 to 190 μm for the bottom electrode, as shown in step (2) of FIG. 21(a). Additional cycles of ablation pattern the surrounding Mg into a helical coil as an inductor and cut the PLA substrate to define the device outline, as shown in step (3) of FIG. 21(a). Assembling these components and sealing the edge between the top and bottom layers of BTP completes the fabrication, as shown in FIG. 21(b). A photograph of the resulting inductor is shown in FIG. 21(c). FIG. 21(d) shows the height profile of the key component determined by 3D confocal optical microscopy, which highlights the height profile of the thinned bottom electrode, as characterized by 3D confocal optical microscopy. The line cut (white dashed) in FIG. 21(d) highlights the thickness variation (190 μm in the bottom electrode area and 280 μm in the inductor area), as shown in FIG. 21(e). The average surface roughness (Ra) and root-mean-square surface roughness (RMS) are ˜4.4 and 5.6 μm, respectively, for the bottom electrode. The quality factor (Q factor) of the inductor is larger than 70, as shown in FIG. 22(b). Inductors with sub-mm scale (diameter: ˜1 mm; number of turns: 4; line width: 90 μm; spacing: 20 μm) dimensions are also possible, as shown in FIG. 22(c) and (d).

FIG. 21(f) shows the working principle of such a device. The schematic cross-sectional illustration as shown in FIG. 21(f) explains the working principle of the device when used as a passive, wireless pressure sensor. The parallel capacitor, with a gap between the top and bottom electrodes defined by the thinning process. The parallel-plate capacitor formed by the air gap between the top and bottom electrodes responds to changes in their separation due to changes in the pressure of surrounding biofluids. Changes in capacitance result from changes in this distance induced by variations in pressure between the surrounding cerebrospinal fluid and the air cavity. The result shifts the resonance frequency of the inductor-capacitor (LC) circuit, which can be detected wirelessly by an external readout coil and vector network analyzer. This LC-type device converts the capacitance change into changes in resonance frequency properties, as the basis for passive, wireless sensing.

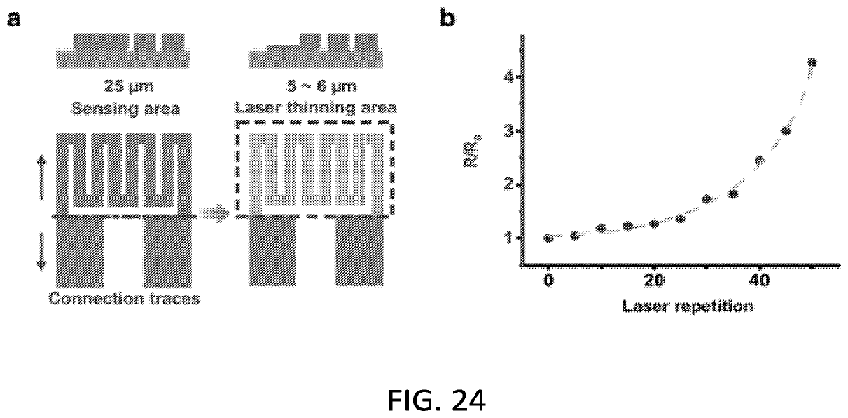

In vivo evaluations in a rat model demonstrate the operation. FIG. 23 shows in vivo acute evaluations in a rat model according to certain embodiments of the invention, where (a) shows a photograph of a bioresorbable physiological monitoring device mounted above an opened craniotomy, with a standard clinical ICP monitor as a reference, and (b)-(d) show acute recordings of physiological parameters, including (b) ICP (red: bioresorbable device; blue: commercial reference), (c) respiration rate and (d) heart rate in the rat model. Specifically, FIG. 23(a) shows the device mounted above an opened craniotomy defect. A standard clinical intracranial pressure (ICP) monitor inserted through a second craniotomy defect serves as a pressure reference. FIG. 23(b)-(d) highlight in vivo acute recordings. Compressing and releasing the flank of the rat cause variations in ICP, as detected by the device (marked red) as shown in FIG. 23(b). The results align with those of the commercial reference (marked blue). The data indicate a sensitivity of ˜500 kHz mmHg⁻¹. Additionally, physiological parameters such as respiration rate (duration: ˜0.7 s) and heart rate (duration: ˜0.2 s), as shown in FIG. 23(c)-(d), can also be tracked in parallel with the ICP measurement.

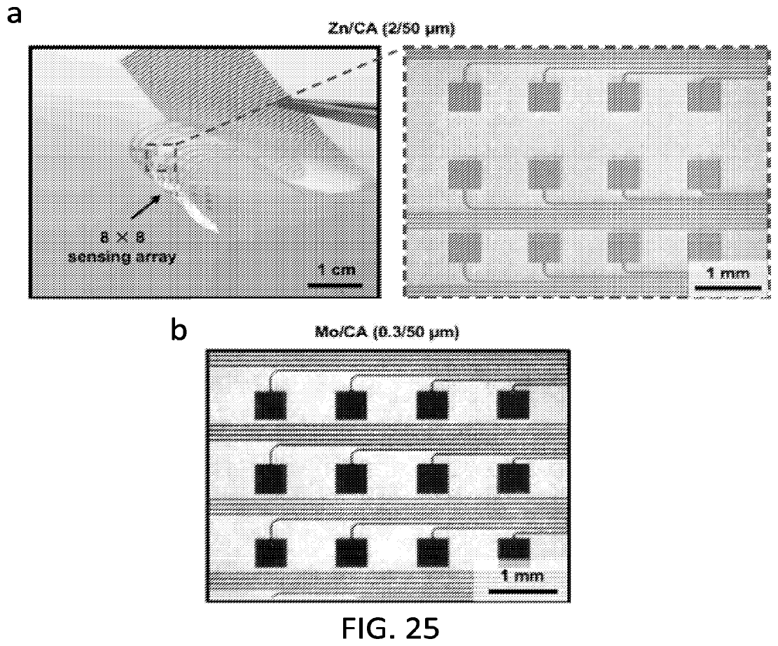

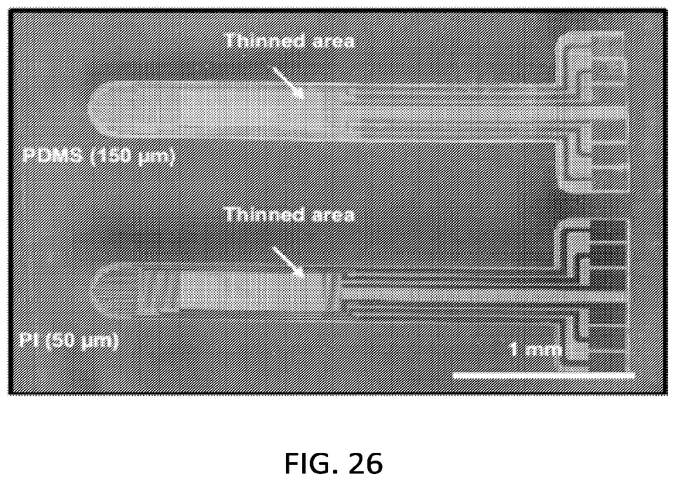

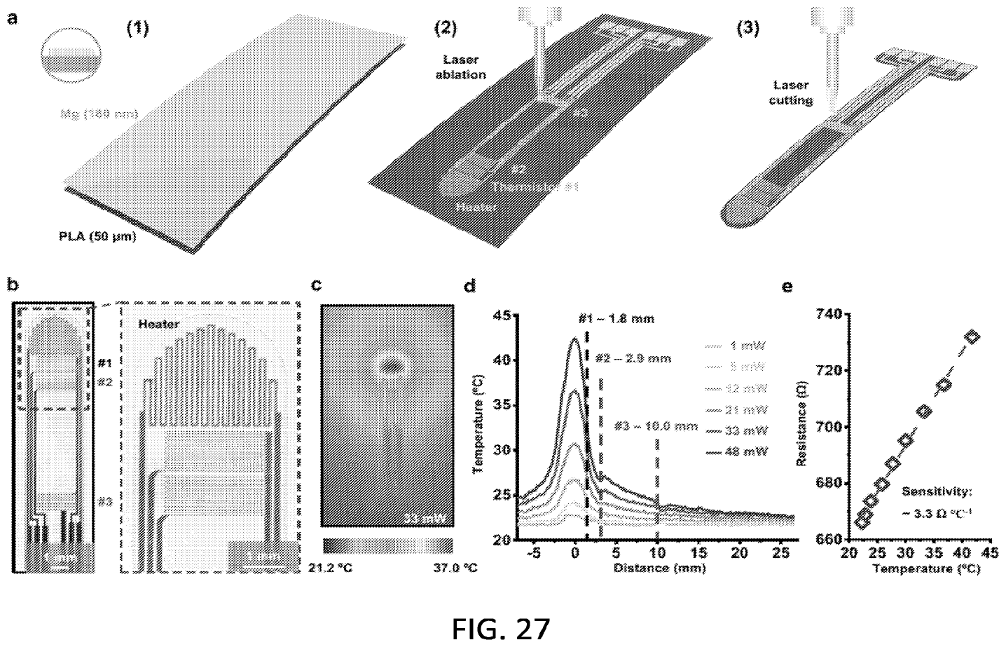

Another demonstration of thickness reduction for resistive-type bioresorbable devices appears in FIG. 24. Specifically, FIG. 24 shows laser thinning strategy for bioresorbable resistive-type devices according to certain embodiments of the invention, where (a) shows the laser thinning method reduces the thickness of the device sensing area from 25 to ˜5-6 μm for high resistance and high sensitivity, while leaving the thicknesses of the connection traces unchanged (thickness: 25 μm), and (b) shows the resistance of the entire device increases by ˜3.5 times when the thickness of the sensing area is reduced from 25 to ˜5-6 μm. The resistance of the sensing area is inversely proportional to the thickness. The same methods can process not only Mg, but also other eco/bioresorbable metals, such as Zn and Mo. FIG. 25 shows laser patterning of (a) Zn and (b) Mo on CA substrates into flexible sensing arrays according to certain embodiments of the invention, which presents results of precise patterning of Zn and Mo on CA substrates into flexible sensing array shapes. Besides eco/bioresorbable materials, representative non-eco/bioresorbable materials, such as polyimide (PI) and polydimethylsiloxane (PDMS), can also be laser-thinned and laser-ablated, as shown in FIG. 26, which shows laser thinning and cutting of non-eco/bioresorbable polymers, including polyimide (PI) and polydimethylsiloxane (PDMS), into probe shapes according to certain embodiments of the invention.

### Processing of Bioresorbable Microvascular Flow Sensing Probes

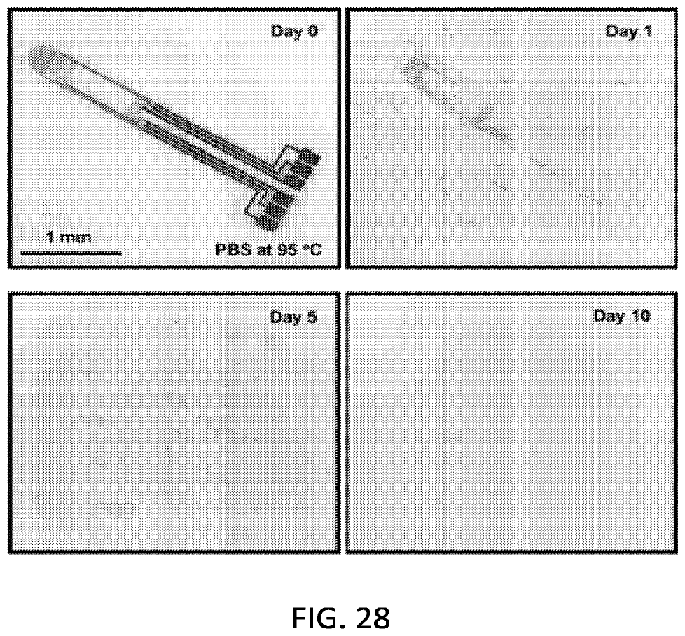

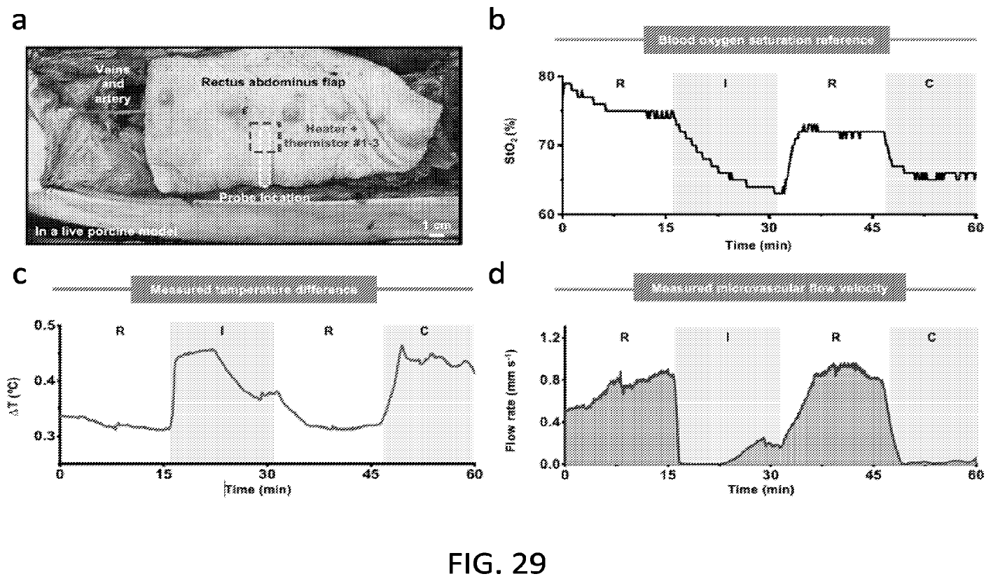

Early detection of thrombosis (blood clotting) in transplants is essential to preserve blood circulation and eliminate serious complications. A class of devices with potential relevance in this context takes the form of a flexible, bioresorbable needle-shaped probe with capabilities in monitoring microvascular flow in soft tissues by using techniques in thermodilution. The laser ablation process can produce such types of probes. The device includes a thin-film heater (material: Mg; thickness: 180 nm; trace width: 50 μm; spacing: 75 μm; resistance: ˜350 Ω) and three thin-film thermistors (material: Mg; thickness: 180 nm; trace width: 18 μm; spacing: 30 μm; resistance: ˜650Ω) each positioned at a different distance to the heater (1.8, 2.9, and 10.0 mm, respectively) and all integrated onto a needle-shaped polymeric substrate (material: PLA; thickness: 50 μm; dimensions: 30×3.5 mm). FIG. 27 shows the process for fabricating bioresorbable microvascular flow sensing probes according to certain embodiments of the invention. Specifically, FIG. 27 (a) illustrates the fabrication procedures, including (1) vacuum deposition of a uniform Mg layer (thickness: 180 nm) on a PLA substrate (thickness: 50 μm), (2) ablation of the Mg layer to define four resistive-type devices, (3) and ablation of the PLA substrate to define a narrow, needle-shaped geometry. Vacuum depositing a uniform layer of Mg (thickness: 180 nm) on a PLA substrate (thickness: 50 μm) prepares the structure for processing, as shown in step (1) of FIG. 27(a). Ablation patterns the Mg layer into narrow traces that form four thin-film resistive-type devices along the length of the substrate, as shown in step (2) of FIG. 27(a). Another ablation process cuts the PLA substrate into a needle-shaped structure, as shown in step (3) of FIG. 27(a). Replacing the Mg layer with a multi-layered stack of SiO₂/Mg/SiO₂ (thickness: 100/180/100 nm) can enhance the performance stability when surrounded by biofluids. Laminating a layer of PLGA (thickness: ˜10 μm) on top and then sandwiching the device with layers of BTP as top and bottom encapsulants complete the fabrication. FIG. 28 shows bioresorption of the laser-fabricated devices according to certain embodiments of the invention, where the device is provided without encapsulation in a solution that approximates the chemistry of biofluids (PBS at 95° C., pH 7.4). As shown in FIG. 28, water-soluble electronic materials can be patterned by laser ablation into devices that harmlessly bioresorb in simulated biofluids, as illustrated here through images of a microvascular flow sensing probe at different times of immersion in PBS at 95° C., pH 7.4 for accelerated testing. The results show that the materials largely dissolve within 5 days and that elimination of residues occurs after 10 days.

FIG. 27(b) shows an optical micrograph of the probe, with a magnified image in the inset to highlight the structures. The inset shows a magnified view of the structures of the heater and thermistors. The results of characterizing the heater and thermistors are shown in FIG. 27(c)-(e). Specifically, FIG. 27(c) shows that the heater produces a temperature distribution around the probe, where an input power of 33 mW generates a maximum temperature increase of ˜16° C. in air., and (e) shows the relationship between the resistance of the thermistor and temperature is linear over this range, with a sensitivity of ˜3.3 Ω° C.⁻¹. FIG. 27(d) shows temperature distribution along with the flow probe at different heater powers. As the heater power rises from 1 to 48 mW, the maximum temperature increases and the heat spreading region expands, as shown in FIG. 27(d). The temperature values at the locations of the three thermistors are different due to the different distances to the heater. FIG. 27(e) shows the relationship between the resistance of the thermistor and temperature is linear over this range. As shown in FIG. 27(e), the thermistors exhibit a proportional relationship between the resistance and temperature, with a sensitivity of ˜3.3 Ω° C.⁻¹.

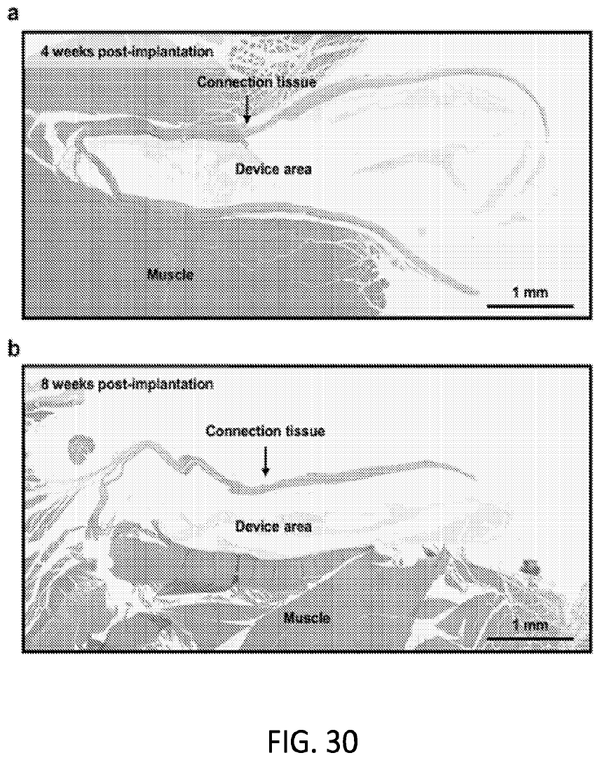

In vivo acute evaluations in a porcine model demonstrate capabilities in accurate, continuous monitoring of microcapillary flow velocity using this device. FIG. 29 shows in vivo acute evaluations in a porcine model using a rectus abdominus myocutaneous flap according to certain embodiments of the invention. Specifically, FIG. 29(a) shows an optical image of the left rectus abdominus flap of a porcine model. The model includes the rectus abdominus myocutaneous flap (dimensions: ˜30×10×2 mm) connected to the surrounding tissues by one main artery and three main veins. Occlusion of the artery and veins leads to ischemia and congestion, respectively. The grey pattern indicates the location of the bioresorbable microvascular flow sensing probe. Clamping the corresponding vein and artery simulates venous (“congested states”, C) and arterial (“ischemia states”, I) thrombosis states, respectively, both of which block capillary blood flow and jeopardize flap viability. R stands for “release state” without clamping vein or artery. FIG. 29(b) shows blood oxygen saturation (StO₂) status of the flap measured by a commercial reference device for different situations (R: release, no occlusion; I: ischemia, artery occlusion; C: congestion, vein occlusion). As shown in FIG. 29(b), in both I and C states, the lack of microcapillary blood flow leads to a drop in StO₂, measured by a commercial oximeter reference mounted on the flap paddle. Inserting the bioresorbable probe into the flap allows measurements of the capillary blood flow rate as shown in FIG. 29(c) and (d). The heater generates thermal power inside the flap, and measurements from the thermistors capture the resulting changes in temperature. The difference between the temperature of adjacent thermistors (ΔT₁₂) is shown in FIG. 29(c). Specifically, FIG. 29(c) shows the temperature difference between two thermistors measured by the bioresorbable device in these situations during operation of the heater, where the temperature differences are ˜0.35° C. in the R state, ˜0.45° C. in the I and C states, respectively. In both I and C states, the temperature differences are large (˜0.45° C.) compared with that in R state (˜0.35° C.). Converting the temperature difference into microcapillary blood flow velocities yields values of 0.8±0.2 mm s⁻¹ in R state, 0.18±0.13 mm s⁻¹ in I state, and 0.05±0.02 mm s⁻¹ in C state, as shown in FIG. 29(d). Notably, the immediate change in the measured flow rate upon entering the I or the C state illustrates capabilities for fast diagnosis and early detection of adverse conditions related to restrictions in blood circulation.

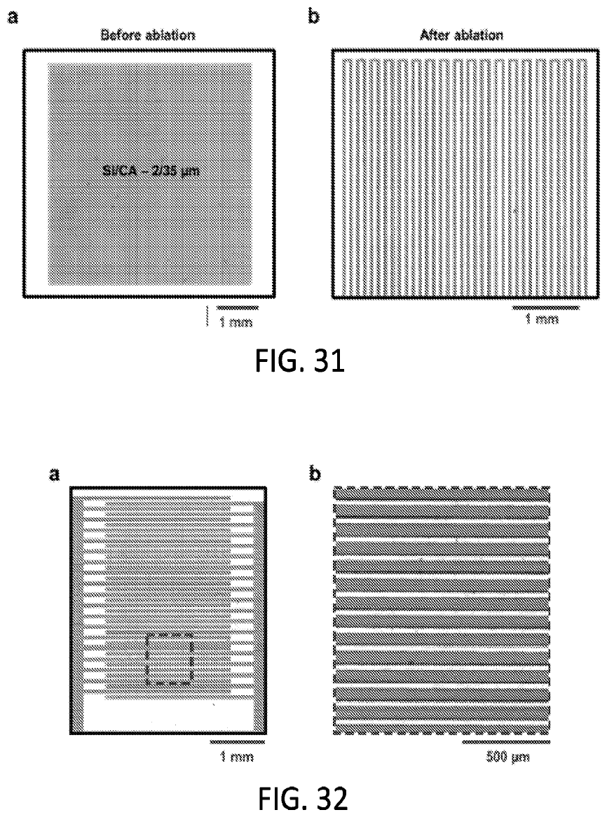

FIG. 30 shows biocompatibility of bioresorbable sensing probes formed by laser ablation according to certain embodiments of the invention. Specifically, FIG. 30 shows images from hematoxylin and eosin (H&E) staining of tissue adjacent to the location of the probe collected from the leg of a rat model at (a) 4 weeks and (b) 8 weeks after implantation. The results indicate the biocompatibility of the devices and the products of their dissolution in biofluids.

### Processing of Multi-Layered Structures for Bioresorbable Si-Based Devices

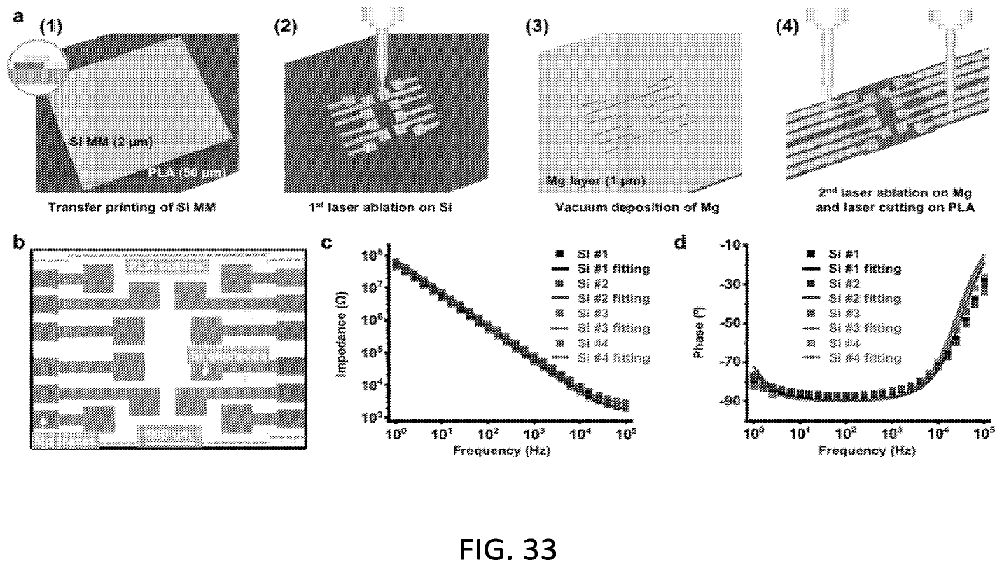

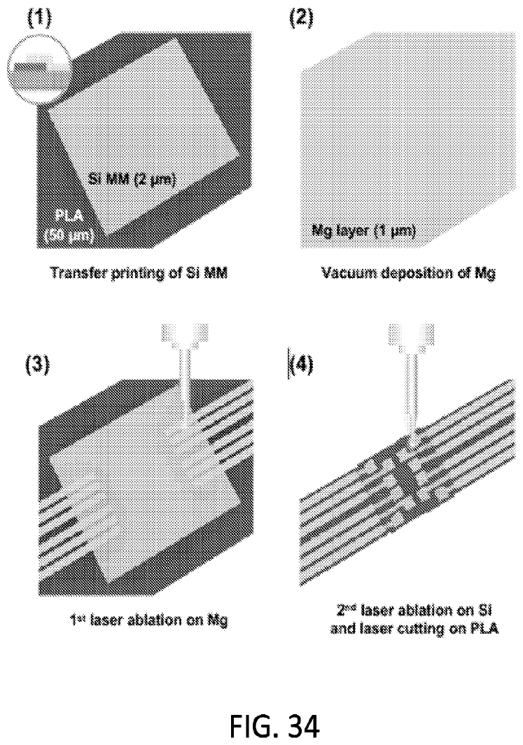

Multi-layered silicon-based electronic architectures can also be realized through multiple cycles of material preparation-patterning. Exemplary devices exploit device-grade, monocrystalline doped Si MMs as the semiconductor for active electronic systems that also include metal interconnection traces and patterned dielectric films. Examples of resistive-type and capacitive-type Si devices are shown in FIG. 31 and FIG. 32. Specifically, FIG. 31 shows laser ablation strategy for Si-based resistive-type devices according to certain embodiments of the invention, where (a) shows an optical micrograph of pristine monocrystalline Si (thickness: 2 μm) on a CA substrate (thickness: 35 μm), and (b) shows an optical micrograph of the ablated monocrystalline Si. FIG. 32 shows laser ablation strategy for forming Si-based capacitive-type devices according to certain embodiments of the invention, where (a) shows an optical micrograph of the interdigitated structure, and (b) shows a magnified image thereof (line width: 100 μm).

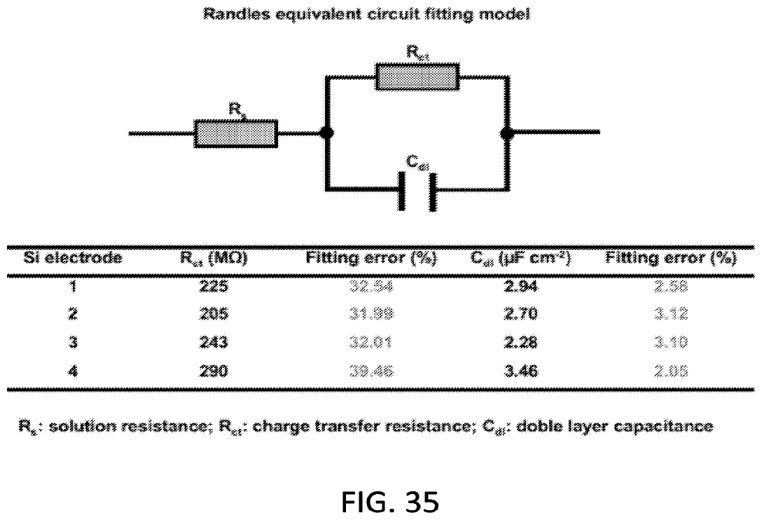

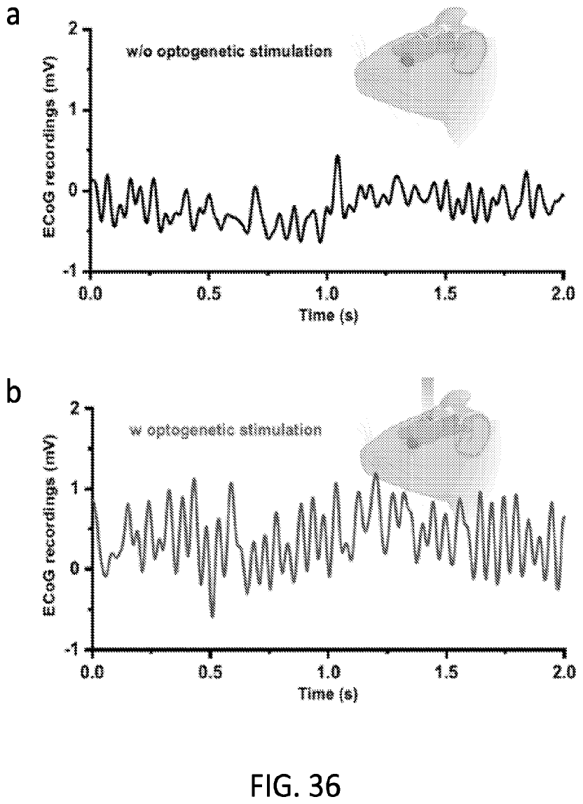

A flexible Si-based bioresorbable multiplexed electrode array using Si MMs as sensing components and films of Mg as interconnection traces serves as a demonstration platform designed for neurophysiologic monitoring of brain function. FIG. 33 shows multi-layer fabrication strategies for Si-based devices and Si-based electrode arrays according to certain embodiments of the invention. Specifically, FIG. 33(a) shows the process to form the multi-layer device, including (1) transfer printing of a monocrystalline highly n-doped Si MM (thickness: 2 μm) on a PLA substrate (thickness: 50 μm), (2) ablation to pattern the Si MM into an array of electrodes, (3) vacuum deposition of a uniform Mg layer (thickness: 1 μm), and (4) ablation of the Mg layer to define connection traces and to cut the PLA substrate into a ribbon shape. Transfer printing a highly n-doped Si MM (thickness: 2 μm) onto a PLA substrate (thickness: 50 μm) defines the initial material structure, as illustrated in step (1) of FIG. 33(a). Ablation patterns the Si MM into electrode shapes (number: 12; dimensions of the electrode pad: 300×300 μm; sensing area in total: 2×2 mm) with interconnection sites (trace width: 120 μm), as shown in step (2) of FIG. 33(a). Another cycle of ablation defines three alignment markers (diameter: 1.5 mm) on the corners of the Si pattern. The second cycle of material preparation-patterning begins with vacuum deposition of a uniform layer of Mg (thickness: 1 μm) on top. Ablation then patterns the Mg into interconnection traces registered to the alignment markers, with minimal damage to the underlying Si layer, as shown in step (3) of FIG. 33(a). Ablation then cuts the PLA substrate into the shape of a ribbon, as shown in step (4) of FIG. 33(a). Laminating a layer of PLGA patterned by ablation on top (thickness: ˜10 μm) encapsulates the system but leaves the Si sensing areas exposed (1.8×1.8 mm), to complete the fabrication. An optical micrograph of such a device appears in FIG. 33(b), with 12 Si electrodes and Mg traces. An alternative laser fabrication process appears in FIG. 34, which shows the alternative process for forming multi-layer Si-based electrode arrays according to certain embodiments of the invention, including (1) transfer printing of a monocrystalline highly n-doped Si MM (thickness: 2 μm) on a PLA substrate (thickness: 50 μm), (2) vacuum depositing a uniform layer of Mg (thickness: 1 μm), (3) ablating the Mg layer to define connection traces, and (4) ablating the Si MM to define an array of electrodes and to cut the PLA substrate into a ribbon shape. This strategy performs ablation after material preparation, as a means to eliminate errors from overlay registration.

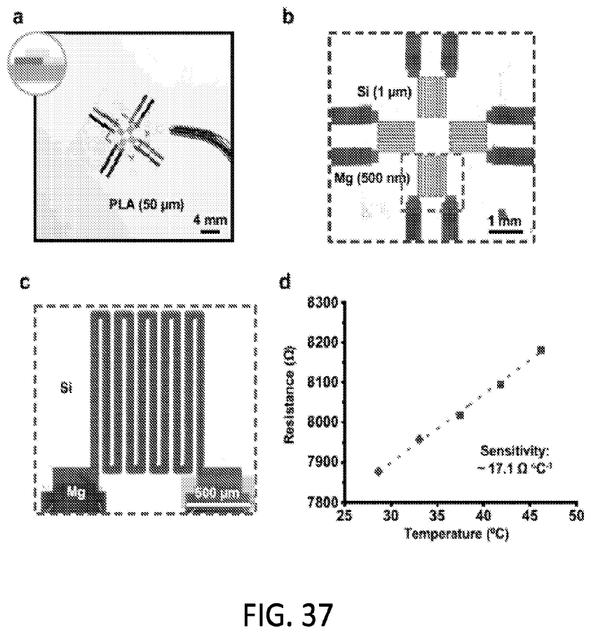

Characterization by electrochemical impedance spectroscopy (EIS) quantifies the performance of these Si electrodes in phosphate-buffered saline (PBS; pH: 7.4) at room temperature. FIG. 33(c) and (d) show electrochemical impedance spectrum (where (c) shows impedance and (d) shows phase angle) of three representative electrodes measured in PBS (pH 7.4) at room temperature (dash line). FIG. 33(c)-(d) correspond to the impedance (|Z|) and phase (°) values. The interface charge transfer resistance, R_{ct}, and double layer capacitance, C_{dl}, are ˜250 MΩ and 2.8 μF cm⁻², respectively, from fits to an equivalent Randles circuit (solid line). At frequencies relevant to neural sensing (˜ 1 kHz), the impedances are ˜50-80 kΩ and the phase angle suggests capacitive behaviors (˜−90°), consistent with related types of Si devices fabricated using conventional schemes. FIG. 35 shows an equivalent circuit model used to fit the electrochemical impedance spectra of Si-based electrodes according to certain embodiments of the invention, where the interface charge transfer resistance, R_{ct}, and double layer capacitance per unit area, C_{dl}, are ˜250 MΩ and 2.8 μF cm⁻², respectively. All these values are in a range consistent with silicon electrodes fabricated by conventional photolithography processes.

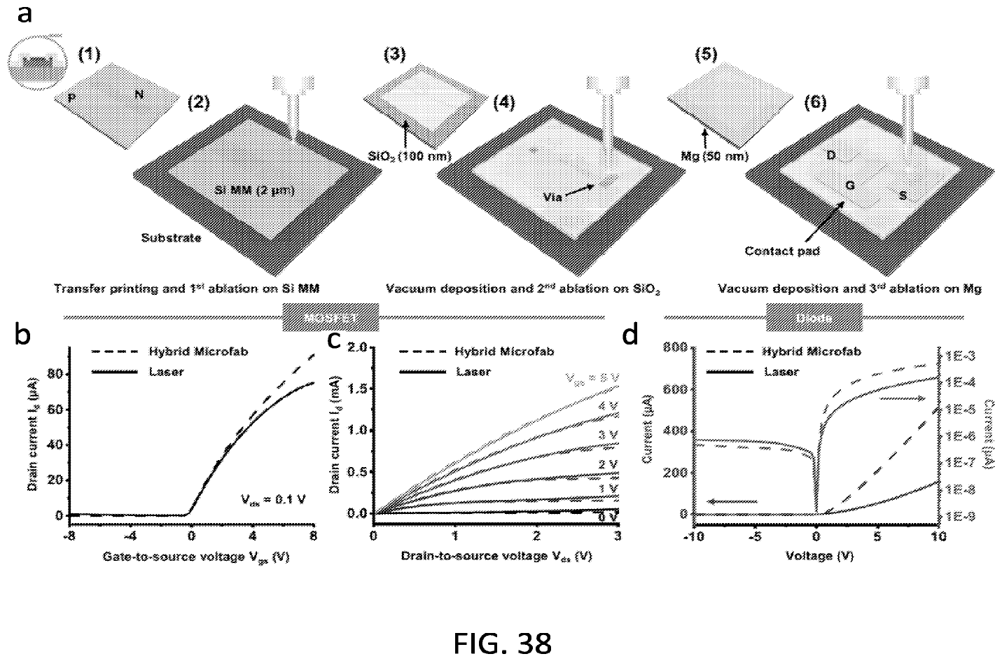

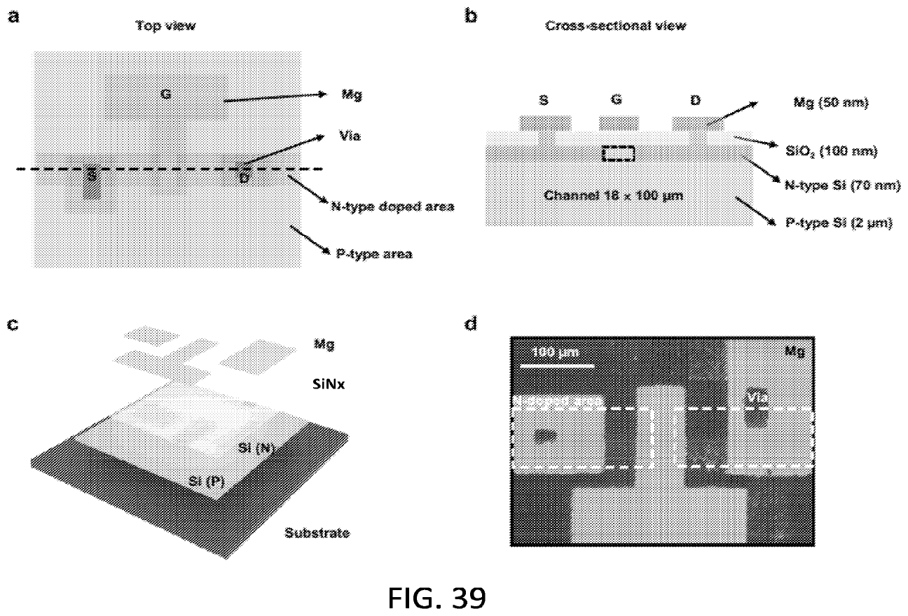

In vivo electrocorticogram (ECoG) recordings in a mouse model illustrate the performance, summarized in FIG. 36. Specifically, FIG. 36 shows in vivo acute optogenetic evaluations in a mouse model according to certain embodiments of the invention, where (a) and (b) show in vivo ECoG recordings in a mouse measured by a representative Si electrode (a) without and (b) with optical simulation. A device and a control electrode lie on the exposed surface of the right primary motor cortex (dimension: 3×3 mm) of an anesthetized mouse (details in methods section). A representative segment (duration: 2 s) of ECoG recordings of sleep spindle activity appears in FIG. 36(a). Optogenetic activation of ChrimsonR by illumination with a light-emitting diode (LED; wavelength: 570 nm; power density: ˜10 mW/mm²) leads to a considerable increment in ECoG signal, as shown in FIG. 36(b), reaching ˜1.0 mV in amplitude when compared to those without optical stimulation (˜0.5 mV). The Si electrode has a thickness of ˜2 μm and partial transparency (˜40% at 570 nm, calculated according to the equation: absorption=log (1/transmittance)) to allow light penetration for co-located optogenetic activation. Similar multi-layer schemes can be used to form other classes of devices, such as Si-based temperature sensors with higher sensitivity than thermistors formed with conventional metals, as shown in FIG. 37. Specifically, FIG. 37 shows multi-layer laser ablation strategy for Si-based temperature sensing arrays according to certain embodiments of the invention, where (a)-(c) show optical micrographs of the device, including four temperature sensors, with highly p-doped monocrystalline Si (thickness: 1 μm) as the sensing element and Mg (thickness: 500 nm) as interconnection traces. The laser ablation procedures are similar to those of the multi-layer Si-based electrode arrays. FIG. 37 (d) shows that the resistance-temperature relation of such a device indicates a sensitivity of ˜17.1 Ω° C.⁻¹.

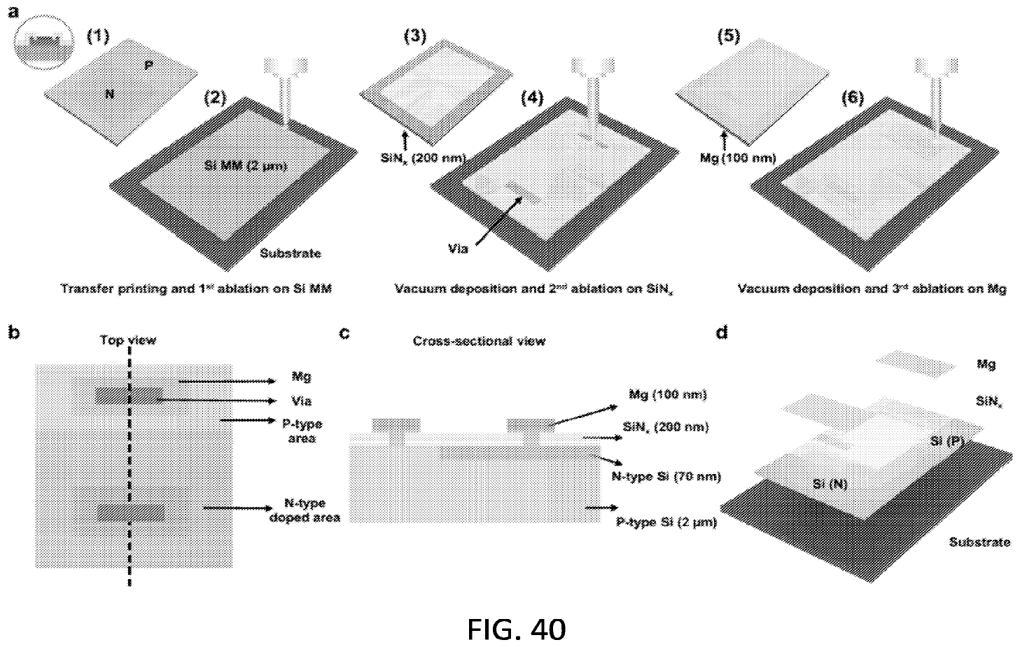

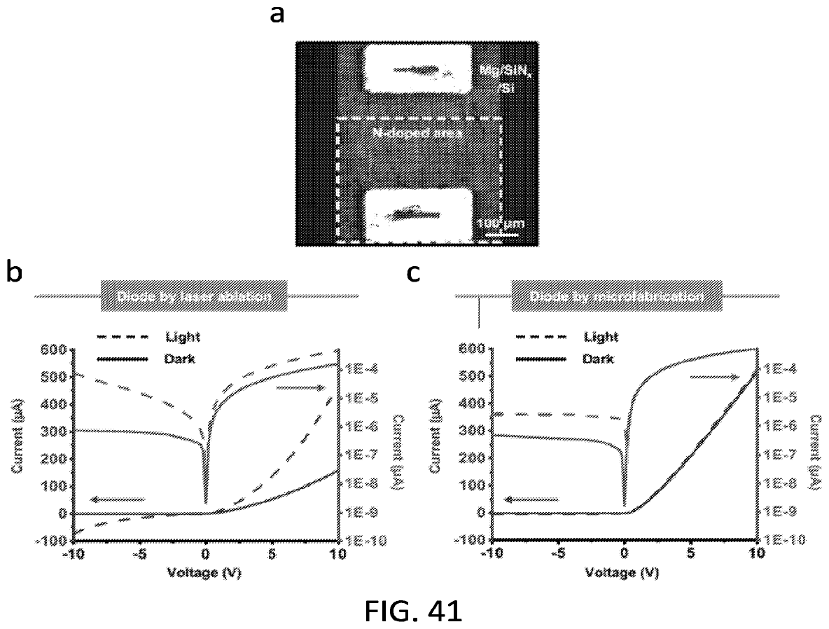

Si-based MOSFETs and diodes are essential elements of advanced eco/bioresorbable electronic systems. Previous publications describe their fabrication based on multiple cycles of photolithography, anisotropic substrate etching, passivation, and transfer printing, with an overall scheme designed specifically to avoid degradation of the bioresorbable constituent materials due to thermal and chemical exposures associated with the processing steps. As an alternative, ablation enables highly simplified routes to similar devices, without compromises in their performance parameters. FIG. 38 shows Si-based MOSFETs and diodes according to certain embodiments of the invention. Specifically, FIG. 38(a) shows the process to form a Si-based n-channel MOSFET as a representative example, formed by three cycles of material preparation-patterning in a sequence similar to that for the ECoG systems mentioned above, including (1) doping of specific regions of a Si MM (thickness: 2 μm), (2) ablation to pattern the Si MM into a rectangular pattern, (3) vacuum deposition of a uniform SiO₂ dielectric layer (thickness: 100 nm), (4) ablation of the SiO₂ layer to define shapes similar to those of Si and with two openings for metal interconnection, (5) vacuum deposition of a uniform Mg layer (thickness: 50 nm), and (6) ablation of the Mg layer to define three connection pads as electrodes. The patterned materials are monocrystalline doped Si (thickness: 2 μm), SiO₂ (thickness: 100 nm), and Mg (thickness: 50 nm). FIG. 39 shows (a) a top view, (b) a cross-sectional view, (c) a 3D exploded view, and (d) an optical micrograph for the multi-layer Si-based n-channel MOSFET devices fabricated by laser methods corresponding to FIG. 38. The bottom Si MM (dimension: 505×515 μm) includes two highly n-doped areas (dimension: 100×248.5 μm), with the other areas lightly p-doped (channel dimension: 18×100 μm). The middle SiO₂ film covers the underlying Si MM, with two openings (dimensions: 40×40 μm and 40×100 μm, respectively) to allow for connection between the Si and Mg pads for electrodes on top. The dimensions for the source, drain, and gate pads are 125×205 μm, 100×135 μm, and 65×180 μm, respectively. FIG. 38(b)-(c) show transfer and output characteristics of devices formed by laser ablation (solid line) and by previously reported schemes (dashed line), with the same materials and dimensions. The effective mobility (μ_{eff}), on-off current ratio (I_{on/off}), threshold voltage (Vₜₕ), and subthreshold slope (SS) values for MOSFETs by laser ablation are 610±20 cm² S⁻¹ V⁻¹, 700±120, −0.35±0.05 V, and 330±60 mV decade⁻¹, respectively; those for devices fabricated by microfabrication are 620±30 cm² S⁻¹ V⁻¹, 3140±2620, −0.30±0.05 V, and 280±50 mV decade⁻¹, respectively. FIG. 38(d) shows IV characteristics of bioresorbable Si diodes with the same materials and the same dimensions fabricated by ablation (solid line) and previously reported approaches (dash line) in linear and log scale. Specifically, FIG. 38(d) compares the characteristics of devices formed by laser ablation (solid line) and previously reported schemes (dashed line), again with the same materials and dimensions. The I_{on/off} and ideality factor (n) for diodes fabricated by laser ablation are 200±60 and 1.6±0.3, respectively; those fabricated by hybrid microfabrication show values of 990±160 and 1.4±0.2, respectively. The differences are likely due to small residual amounts of Mg.

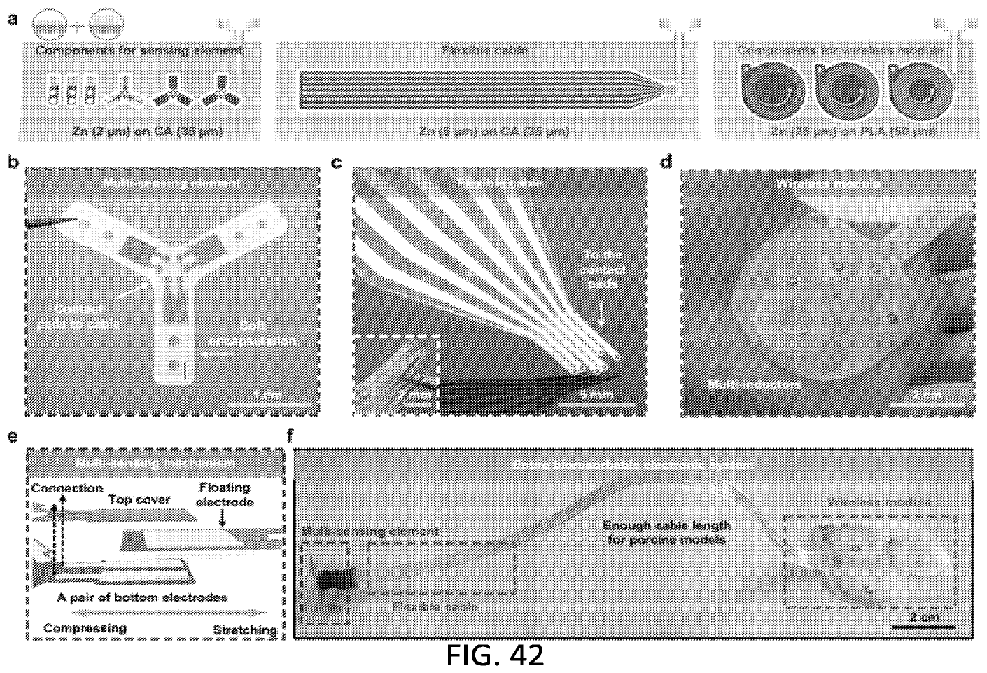

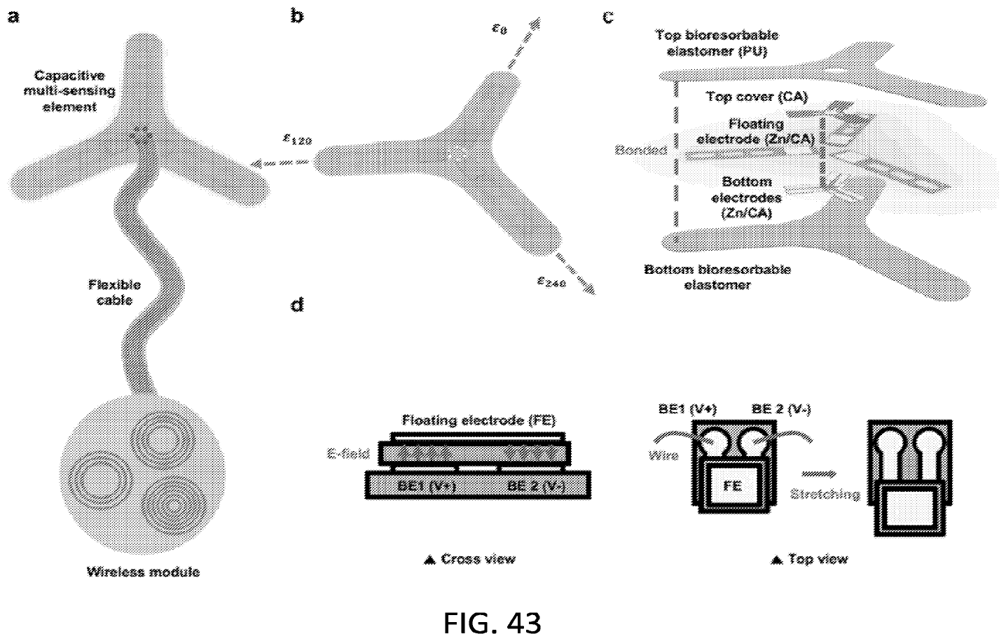

Similar procedures can produce diodes, as illustrated in FIG. 40 and FIG. 41. Specifically, FIG. 40(a) shows the laser ablation process to form a multi-layer Si-based diode, including (1) doping a specific region (dimension of highly n-doped area: 500×500 μm) on a monocrystalline Si MM (thickness: 2 μm), (2) ablation to pattern the Si MM into a rectangular pattern (dimension: 500×800 μm) into the PN junction pattern, (3) vacuum deposition of a uniform SiNx dielectric layer (thickness: 200 nm), (4) ablation of the SiNx layer to define shapes similar to those of Si and with two exposed vias (dimension: 50×140 μm), (5) vacuum deposition of a uniform Mg layer (thickness: 100 nm), and (6) ablation of the Mg layer to define two connection pads (dimension: 155×280 μm). FIG. 40(b)-(d) shows different views of the diode fabricated by the laser ablation, and FIG. 41(a) shows an optical micrograph of the diode fabricated by laser ablation. FIG. 41(b)-(c) show photoresponse characteristics thereof, where the intensity of the white LED used in the light environment is 10,000 lux. As expected, these devices exhibit a photoresponse, comparable to that of devices formed by hybrid microfabrication, as shown in FIG. 41(b)-(c). As shown in FIG. 14(b), some debris with characteristic sizes of ˜1 μm appears on the surface of the ablated polymeric substrate, and that with sizes of ˜100 nm appears on both the Si ribbon and polymeric substrate. According to the observed performance of the MOSFETs and diodes, this particulate debris has no significant effect on the operation of fabricated devices.

### Integrated Processing for Wireless Bioresorbable Systems for Cardiac Monitoring

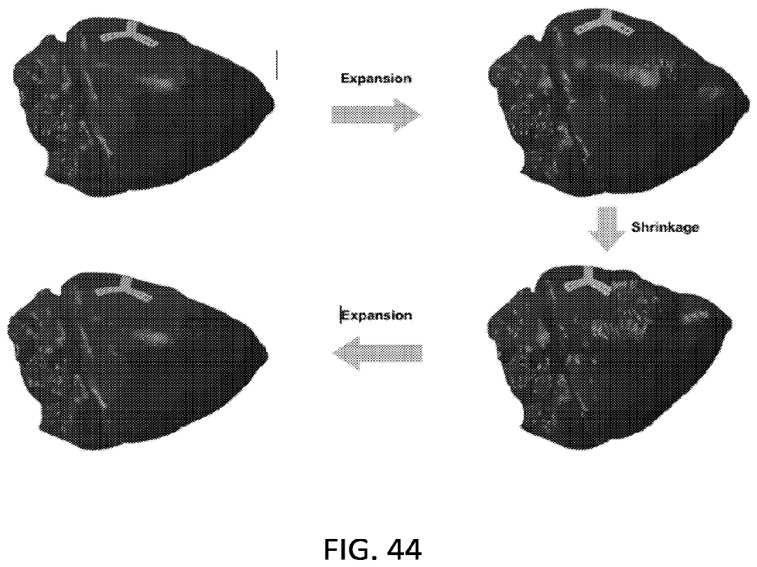

Monitoring of mechanical strains associated with cardiac activity can be valuable in the diagnosis of cardiac disorders and early detection of cardiac arrest. In an example relevant to this area of medical monitoring, an integrated processing sequence yields a type of wireless, bioresorbable, mechanically compliant, multi-sensing electronic system for measuring principal and shear strains on the surface of the heart. A unique device of this type, formed by laser ablation processes, consists of a multi-sensing capacitive element, a flexible cable with six connection traces, and a wireless module with three in-plane inductors, as illustrated in FIG. 42. Specifically, FIG. 42 shows laser ablation for integrated wireless bioresorbable cardiac systems according to certain embodiments of the invention, where FIG. 42(a) shows the components used to construct the cardiac devices, and FIG. 42(b)-(d) show photographs thereof. In particular, FIG. 42(b) shows a photograph of the multi-sensing element. As shown in FIG. 42(b), The multi-sensing element (FIG. 6b) includes several components, including, from bottom to top: a bottom WPU encapsulation (thickness: ˜100 μm), a tri-arm Y-shaped layer with three pairs of bottom electrodes (material: Zn/CA; thickness: 2/35 μm; dimension of each arm: 6.9×3.1 mm), three single-arm floating electrodes (material: Zn/CA; thickness: 2/35 μm; dimension: 10.9×3.1 mm), a tri-arm Y-shaped top cover (material: CA; thickness: 35 μm; dimensions of each arm: 6.9×3.3 mm), and another top WPU encapsulation. The hole on each arm close to the center aids device mounting with a customized accessory. The hole close to the end facilitates suturing.

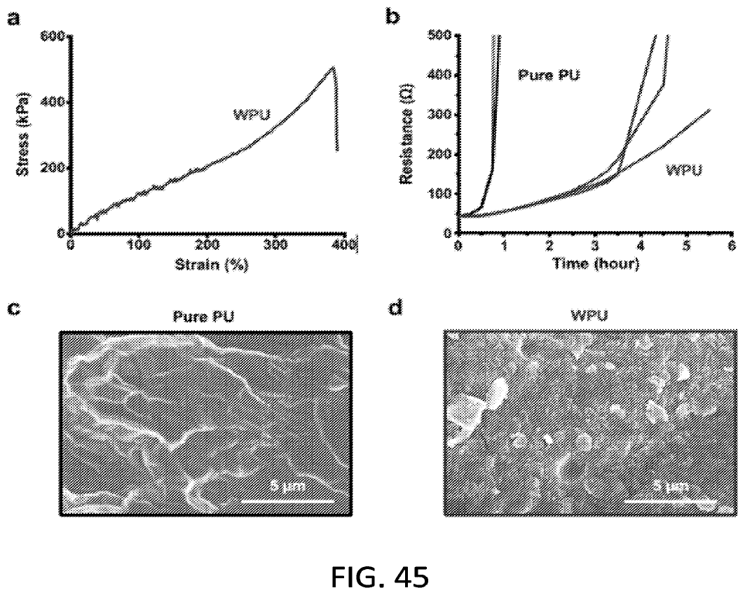

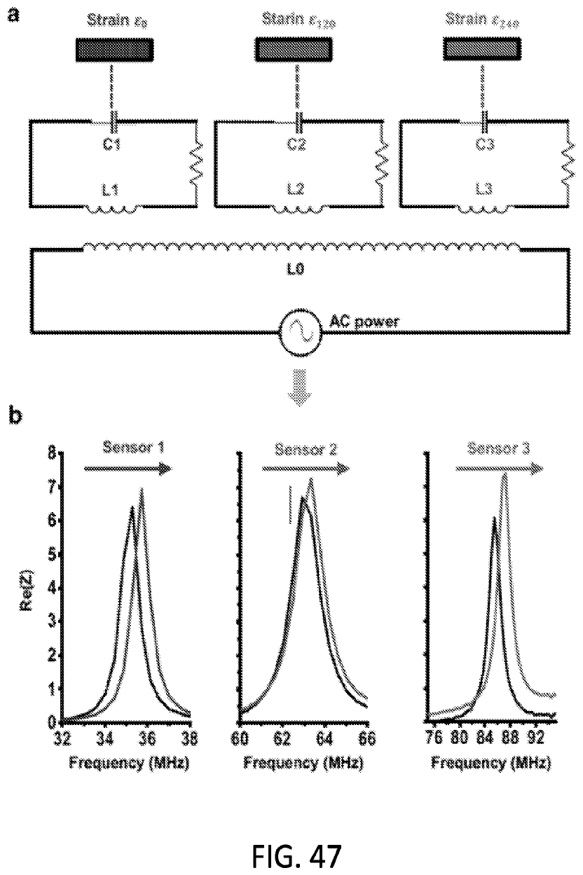

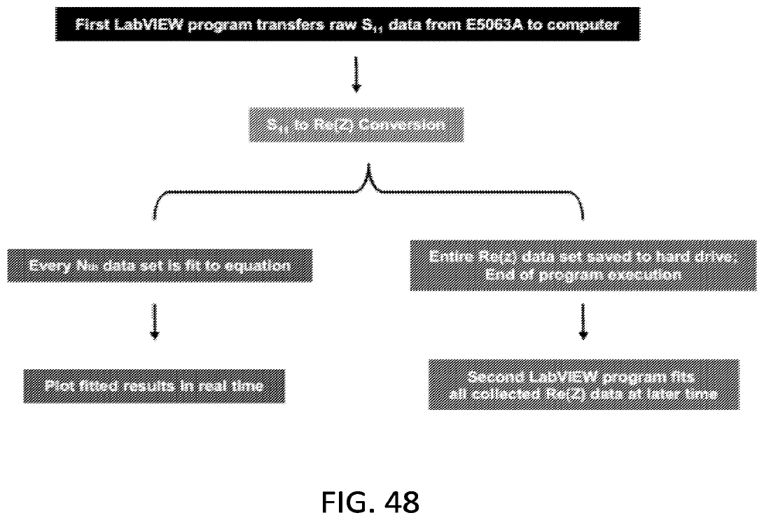

Details of the components of the multi-sensing element as shown in FIG. 42(b) are shown in FIG. 43(a)-(c). Specifically, FIG. 43 shows device architectures and working principles of the multi-sensing element as shown in FIG. 42, where (a) shows multi-sensing element connects to the wireless module by a long flexible cable, (b) shows the element measures strains along the three arms, (c) shows an exploded view of the element, including three functional layers (bottom electrodes, floating electrodes, and top cover) and two encapsulation layers, and (d) shows the working principles for strain sensing. Two uniform layers of bioresorbable wax-based polyurethane (WPU) elastomers (Young's modulus: ˜100 kPa; see FIG. 45(a)) act as top and bottom encapsulation layers (thickness: ˜100 μm; dimensions of each arm: 15.0×5.0 mm). Specifically, FIG. 45 shows tissue-like mechanical and water barrier encapsulation properties of the WPU layer according to certain embodiments of the invention, where (a) shows the stress-strain curve for the WPU indicates a tissue-like Young's modulus of ˜100 kPa, and (b) shows relative water permeation properties of WPU and pure PU, determined by the time dependence of the resistance of a thin-film Mg trace. Water that penetrates through the encapsulation layers dissolves the Mg traces, and therefore, increases the resistance. WPU has superior water barrier characteristics compared to those of pure PU. FIG. 45(c)-(d) show scanning electron microscope (SEM) images of the cross-section of (c) pure PU and (d) WPU. The WPU elastomers, based on dynamic covalent polyurethanes, support improved water barrier properties through the addition of natural wax, without compromise in mechanical properties, as shown in FIG. 45(b)-(d). Stable recording is possible for several hours in vivo. Enhancing the water barrier properties of the encapsulation material is critical to increasing the functional lifetime of the device. The hole closest to the center of the device on each arm facilitates device manipulation with a custom 3D-printed attachment, as shown in FIG. 46. Specifically, FIG. 46 shows customized 3D-printed accessory to hold the device in a manner that avoids bending and fracture during the surgery according to certain embodiments of the invention, where (a) shows a schematic illustration of the accessory, and (b) shows an optical image of the accessory to hold the device on the cardiac surface. The hole close to the end allows for suturing to underlying cardiac tissues.

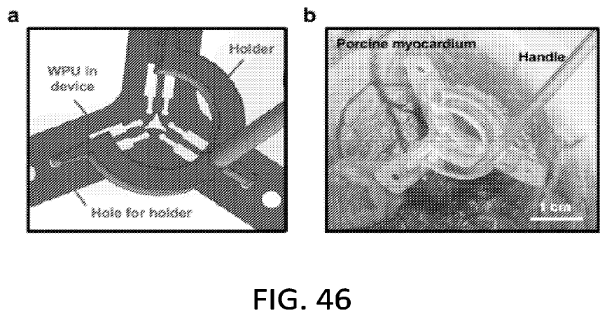

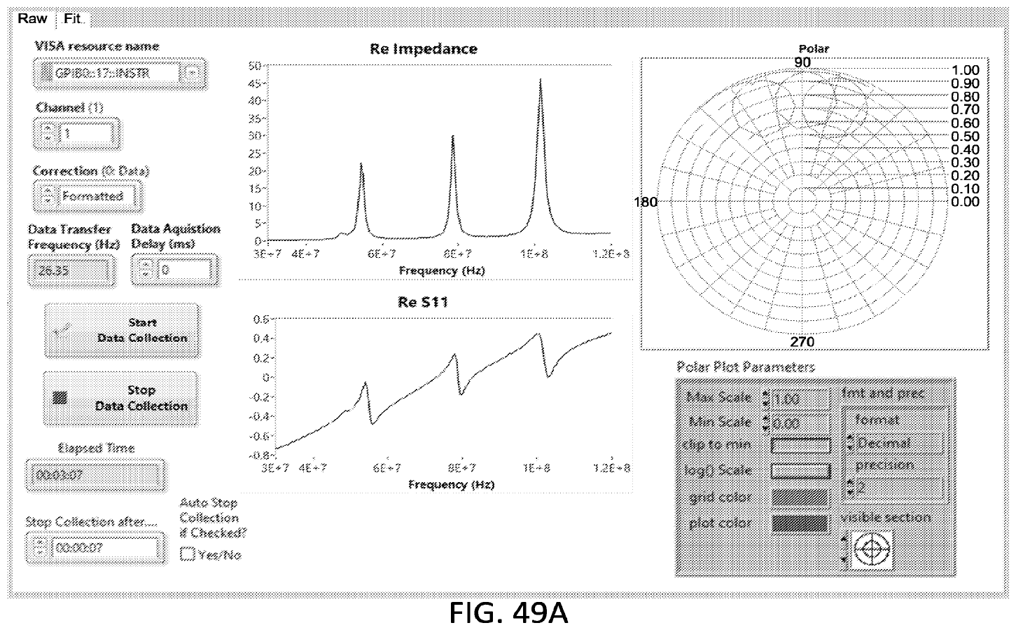

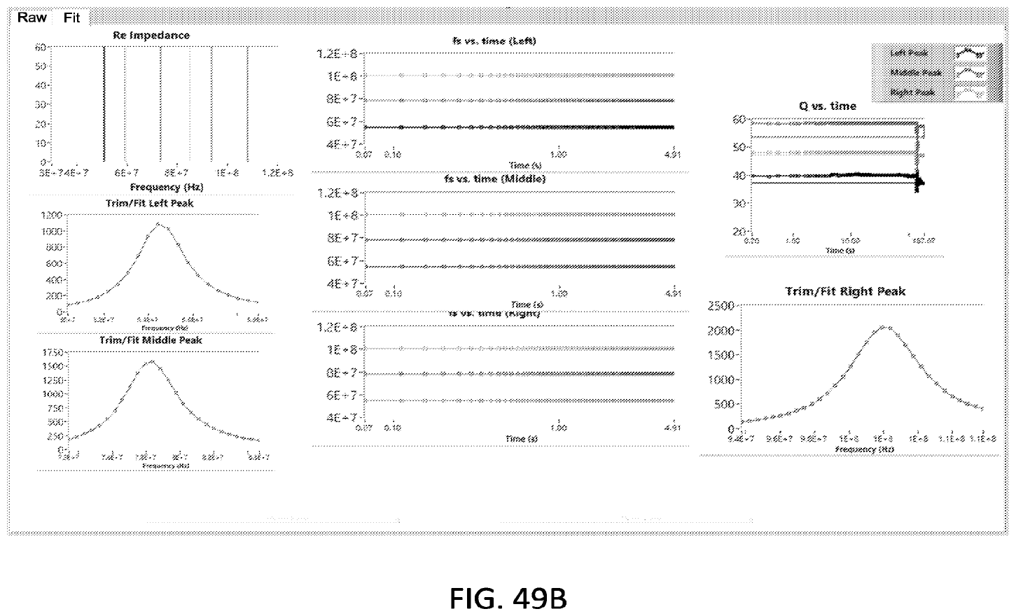

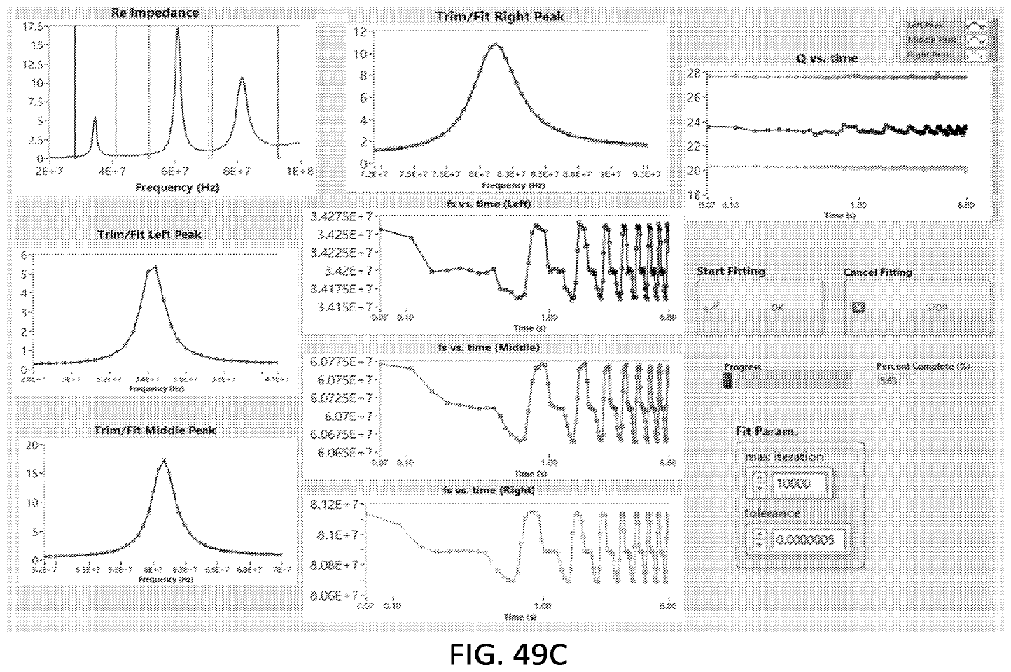

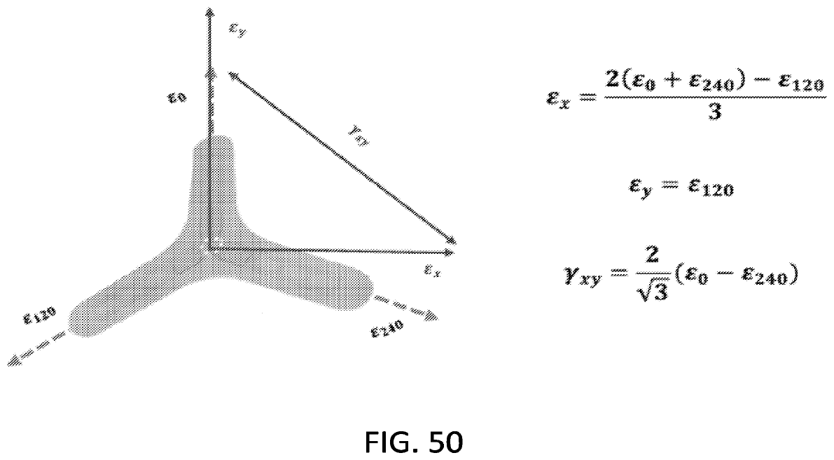

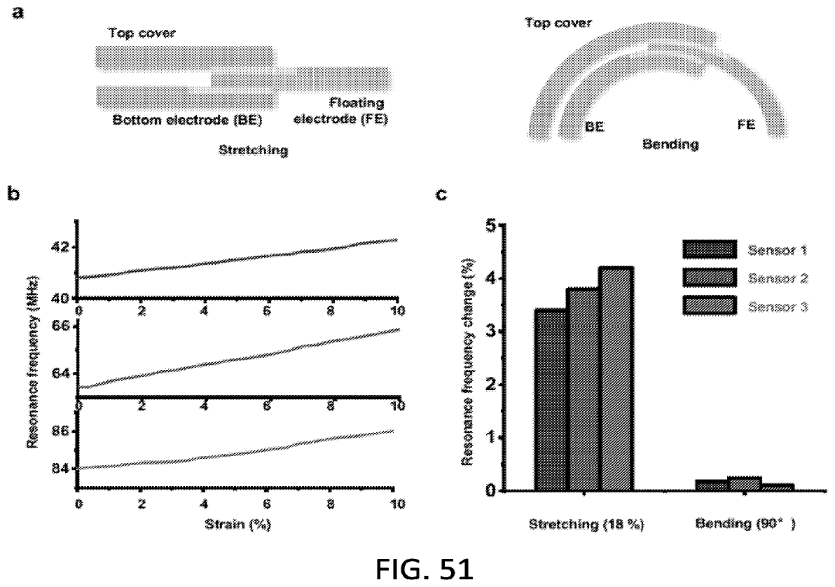

FIG. 42(c) shows a photograph of the flexible cable, which includes six independent Zn traces (thickness: 5 μm; trace width: 1 mm; spacing: 1.5 mm) on a cellulose acetate (CA) polymeric substrate (thickness: 35 μm; width: 14 mm), with a uniform PLGA layer as the top encapsulation (thickness: ˜10 μm). FIG. 42 (d) shows a photograph of the wireless module. The wireless module as shown in FIG. 42(d) includes three Zn inductors (thickness: 25 μm; line width: 200 μm; spacing: 500 μm; outside diameter: 19.6 mm; number of turns: 4, 6, and 10) on a PLA substrate (thickness: 50 μm) with different inductance values to achieve different resonance frequencies for each of the three capacitive sensors in the multi-sensing element. Two uniform PLGA layers act as the top and bottom encapsulation structures (thickness: 50 μm). Stretching each arm of the multi-sensing element along its axis leads to a change in the relative position between the floating electrode and the pair of bottom electrodes. FIG. 42(e) shows the working principle of the device. The strain along each arm leads to a change in the relative position between the floating electrode and the pair of bottom electrodes, and therefore, this change in position leads to a corresponding change in capacitance. The change in capacitance results in a shift in resonance frequency. In other words, the result is a corresponding change in the capacitance between the pair of the bottom electrodes, as shown in FIG. 42(e) and FIG. 43(d), as well as FIG. 44, which shows expansion and contraction of the heart during each cardiac cycle leads to epicardial strains that can be measured by the multi-sensing element according to certain embodiments of the invention. These changes alter the resonance frequencies of the LC oscillators along each arm, as shown in FIGS. 47, 48 and 49A-49C. Specifically, FIG. 47 shows an equivalent circuit model for simultaneous wireless measurements according to certain embodiments of the invention, where (a) shows an equivalent circuit of the three LC-resonance sensing units in a multi-sensing element (top) and the wireless readout system (bottom), and (b) shows stretching the arms leads to a reduction of the capacitance and an increase in the resonance frequency. FIG. 48 shows a flowchart for a custom Lab VIEW program for real-time data collection and analysis and subsequent further analysis according to certain embodiments of the invention. FIGS. 49A and 49B show custom Lab VIEW program for real-time data collection and analysis and subsequent further analysis, where the program collects and analyzes the reflection coefficient (S11) data measured with the ENA network analyzer in real time and converts the S11 data to the real part of the impedance (Re(Z)) accordingly, with further calculations on the resonance frequency (fₛ) and Q factor (Q) in real time, albeit with low time resolution (maximum data frequency: ˜5 Hz; lower than the data collection frequency (˜25 Hz)). FIG. 49C shows that the custom LabVIEW program also post-processes all the data. These changes in frequency can be converted to the principal strains (εₓ and ε_{y}) and shear strain (γ_{xy}) by coordinate transformation. Specifically, FIG. 50 shows coordinate transformation to convert the strains along 0°, 120°, and 240° for each arm to principal strains (εₓ and ε_{y}) and shear strain (γ_{xy}) according to certain embodiments of the invention. FIG. 42(f) shows a photograph of the complete device for measuring strains on the surface of cardiac tissue in large animals.

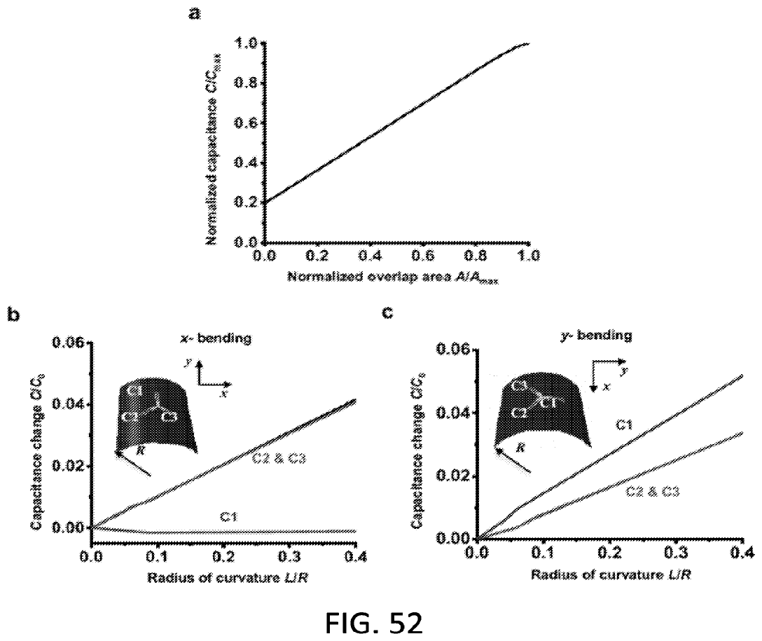

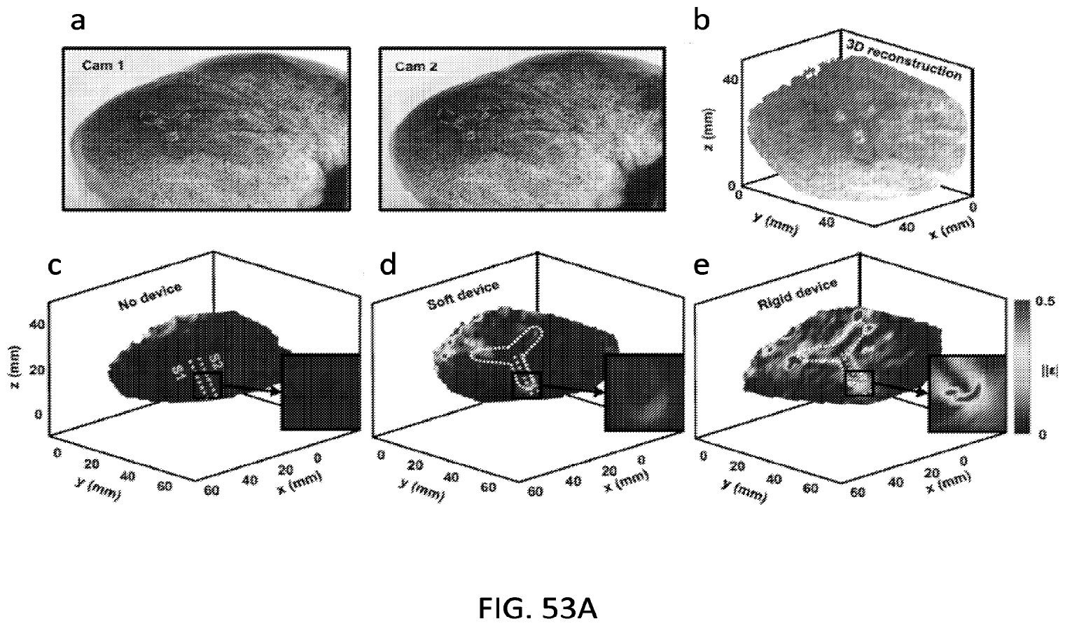

Characterization of the device reveals high strain sensitivity, negligible bending sensitivity, and minimal mechanical load on the underlying tissue. The variance of the resonance frequency in each arm is ˜3-4% at a maximum strain of ˜18%, indicating a sensitivity of ˜200 kHz per unit strain, as shown in FIG. 51. Specifically, FIG. 51 shows strain sensitivity and bending insensitivity of the multi-sensing element according to certain embodiments of the invention, where (a) shows functionality of the top cover. The top cover fixes the distance between the floating electrode and the pair of bottom electrodes; and therefore, the capacitance remains stable under curvature. FIG. 51(b) shows change in resonance frequency for each arm is ˜1.5-2% with a strain of 10%, suggesting a sensitivity of ˜200 kHz per unit strain, and FIG. 51(c) shows the change in resonance frequency is ˜3-4% with a strain of ˜18% and <0.2% under a bending angle of 90°, respectively, suggesting negligible sensitivity to bending. The tri-arm Y-shaped top cover fixes the distance between the floating electrode and the pair of bottom electrodes. In this way, the capacitance remains largely unchanged by bending deformations. FIG. 52 shows simulation results for the sensitivity to strain and bending according to certain embodiments of the invention, where (a) shows normalized capacitance exhibits a linear relationship with normalized overlap area/length (with fixed width) during stretching, and (b)-(c) show relationships between the capacitance change and the radius of curvature normalized by the device in-plane size L for the three capacitors as a result of x-and y-bending. The capacitance change under curvature is negligible compared with that under stretching. Even at a bending angle of 90°, the change in the resonance frequency of each arm is smaller than 0.2%, negligible compared to changes associated with stretching through a cardiac cycle, as shown in FIG. 51(c), and consistent with simulation results as shown in FIG. 52.

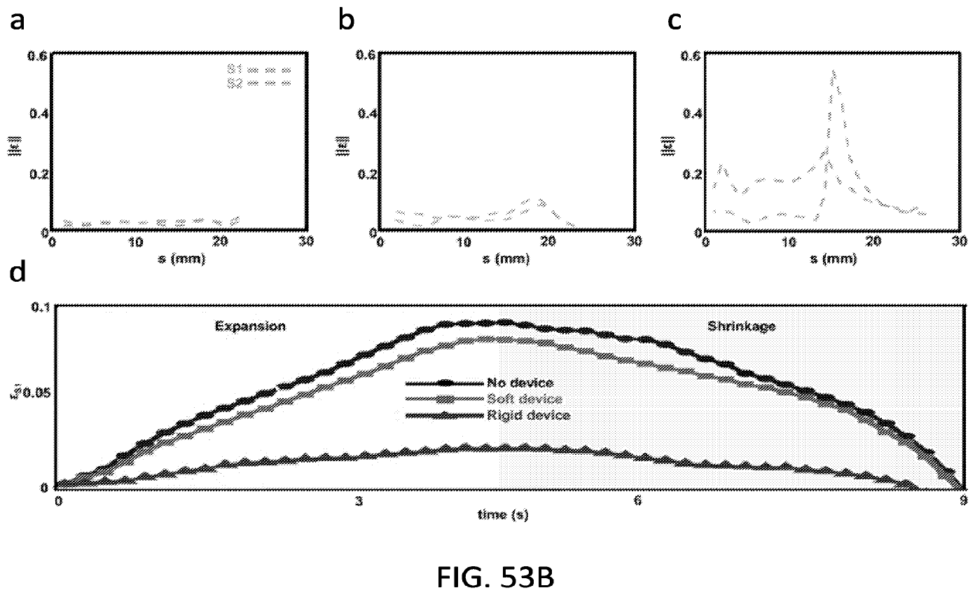

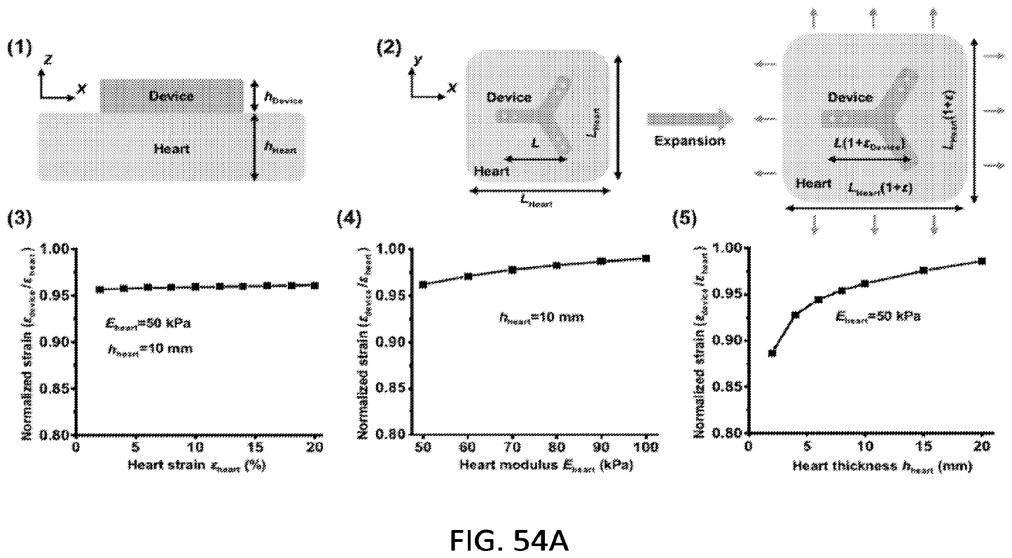

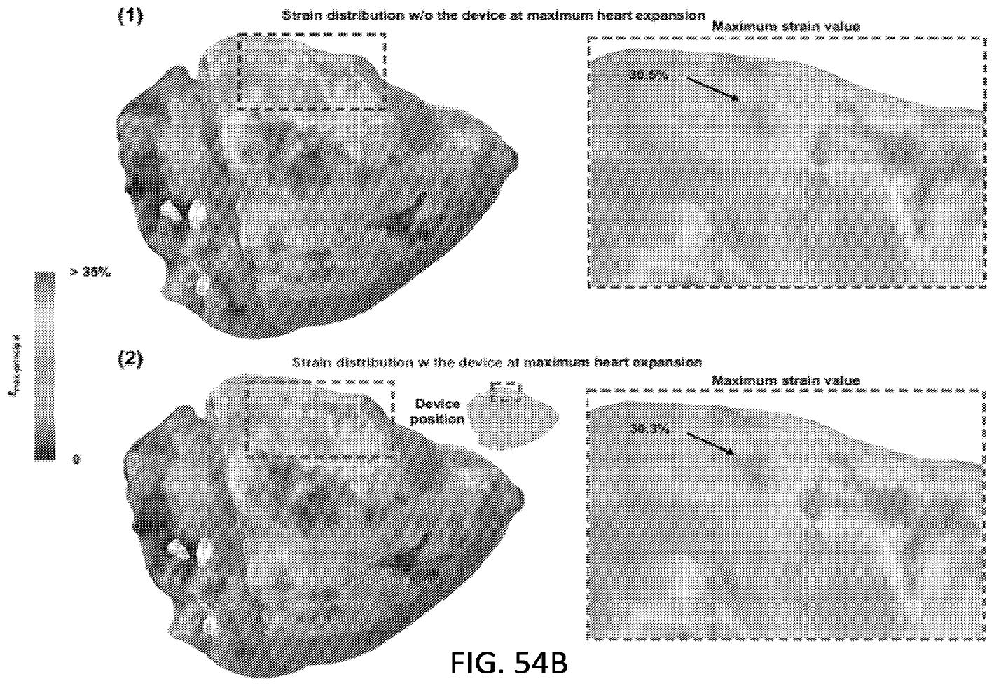

Characterization by 3D-particle tracking velocimetry (3D-PTV) proves that the device is mechanically compliant to the cardiac tissues with negligible confinement. The strain distributions on the same position of the porcine cardiac surface without and with a mounted device during the cycles of expansion and contraction (duration: 4.5 s for each stage) appear in FIG. 53A and FIG. 53B. Specifically, FIG. 53A shows insignificant mechanical constraints on natural motions of the cardiac tissues according to certain embodiments of the invention, where FIG. 53A (a) shows optical images from the two cameras for 3D-PTV. As shown in FIG. 53A(a), the porcine heart surface has many plastic circular black particles (diameter: 1 mm) for 3D-PTV. FIG. 53A(b) shows 3D reconstruction of the cardiac tissue with device mounted, and FIG. 53A(c)-(e) show strain distributions of the cardiac surface without the device, with the device, and with a rigid film in a similar structure (Young's modulus: ˜1 GPa) at maximum heart volume. FIG. 53B shows additional mechanical constraints of FIG. 53A, where FIG. 53B(a)-(c) show quantitative assessments of the strain values (S1 and S2) along two lines in FIG. 53A(c)-(e) at full heart volume. In the cases without and with the device, the strain values are all lower than 0.1. By contrast, the strain values are much higher (maximum value over 0.5) for the case of a uniform film. FIG. 53B(d) shows strain values (S1) during the entire expansion-contraction process. The deformations with the device (strain at the full volume: ˜8%) are similar to those without the device (strain at the full volume: ˜9%). By contrast, the attachment of a uniform film reduces the strain at full expansion to ˜2%. The deformations with the device (strain at the full volume: ˜8%) are similar to those without the device (strain at the full volume: ˜9%). By contrast, the strain at the full expansion decreases ˜2% as a result of mounting a uniform film in a similar shape (Young's modulus: ˜1 GPa) on the cardiac surface. The mechanical compliance of the device corresponds well with simulation results as shown in FIG. 54A and FIG. 54B and allows accurate recordings of natural cardiac deformations. In particular, FIG. 54A shows results using a simplified model on the constraints to natural motions of cardiac tissues, where (1) shows a cross-sectional view of the device on the cardiac tissues, (2) shows a top view of the device on the cardiac tissues, (3) shows normalized strain (ε_{device}/εₕₑₐᵣₜ, which is the device strain ε_{device} divided by the heart strain εₕₑₐᵣₜ) as a function of heart strain, (4) shows the normalized strain as a function of heart modulus, and (5) shows the normalized strain as a function of heart thickness. These results suggest a negligible influence of the device on motions of the heart. FIG. 54B shows results using a living heart model, where (1) shows strain distribution without the device and (2) shows strain distribution with the device at maximum heart expansion. The insets in FIG. 54B show maximum strain values at maximum heart expansion (1) without the device and (2) with the device are 30.5% and 30.3%, respectively, suggesting negligible difference.

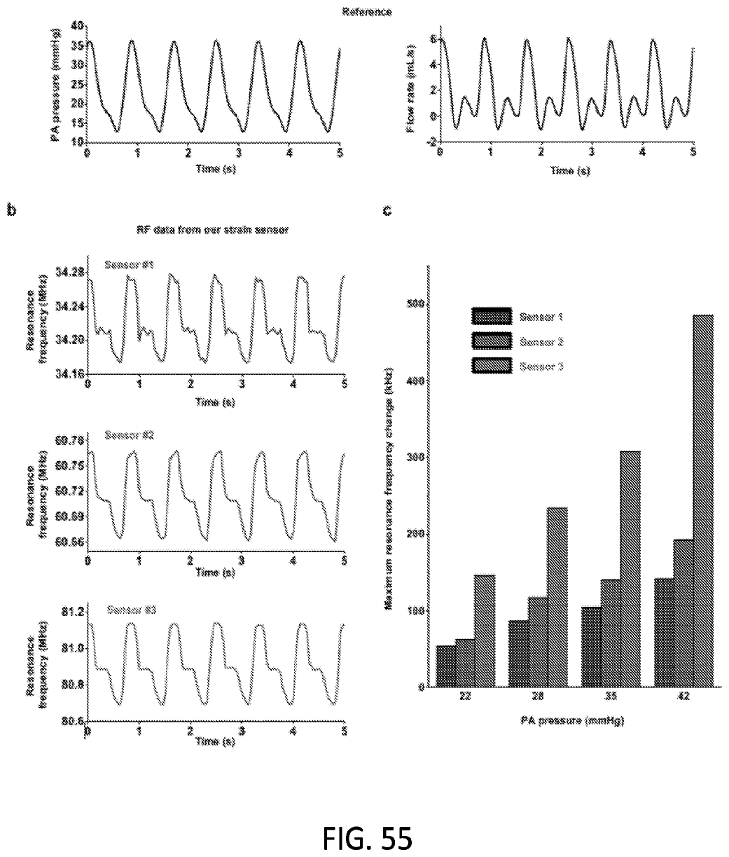

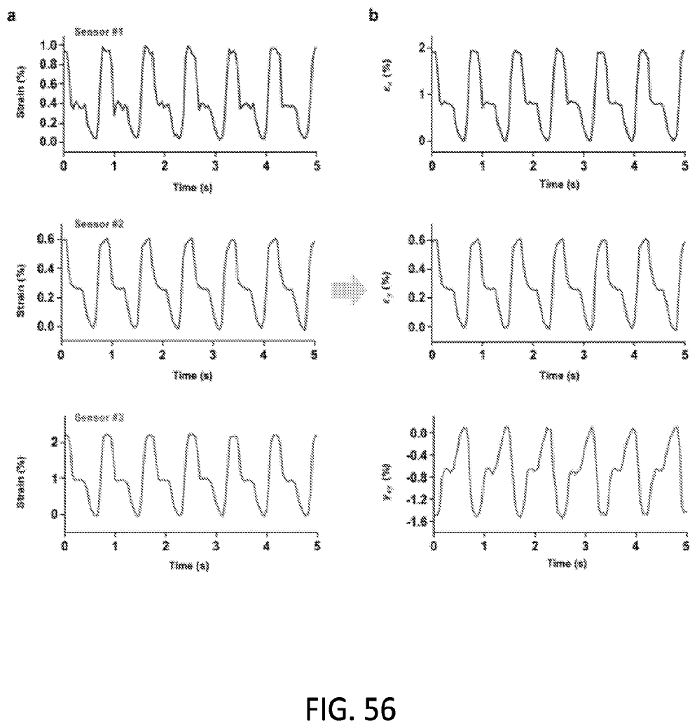

Ex vivo and in vivo evaluations highlight the functionality. The ex vivo studies utilize an artificial heart system with adjustable and oscillatory flow and pressure. Mounting the device on the right ventricle (RV) of a porcine heart with three suture sites on the ends of each arm allows the compliant deformation of the device for mounting on the heart. FIG. 55 shows ex vivo evaluations of the cardiac device on an artificial heart system with adjustable oscillatory flow and pressure, where FIG. 55(a) and (b) present the resonance frequency for the three sensing units, each of which follows the reference pressure and flow rate data in terms of frequency and qualitative waveform shapes. Specifically, FIG. 55(a) shows PA pressure and flow rate as references, and FIG. 55(b) shows the resonance frequency data for each sensing unit. The results correspond well to the reference pressure and flow rate data in terms of waveform shape and frequency. FIG. 55(c) shows the maximum change in resonance frequency increases as the PA pressure rises in these three sensing units. The maximum change in frequency increases as the pressure in the pulmonary artery (PA) rises. Converting the frequencies to strains along each arm, and then analyzing the strains according to the coordinate transformation determines the principal and shear strains of the local position, as shown in FIG. 56, which shows principal and shear strains in ex vivo evaluations of the cardiac device. Specifically, FIG. 56(a) shows converted strain data along with each sensing unit, and FIG. 56(b) shows coordinate transformation of the strain values along the three arms determines the principal and shear strains.

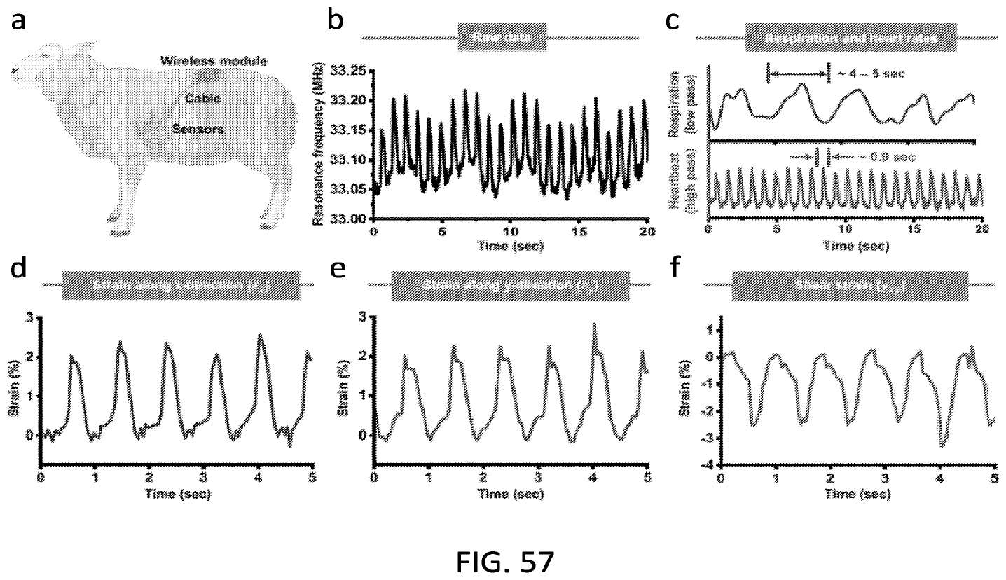

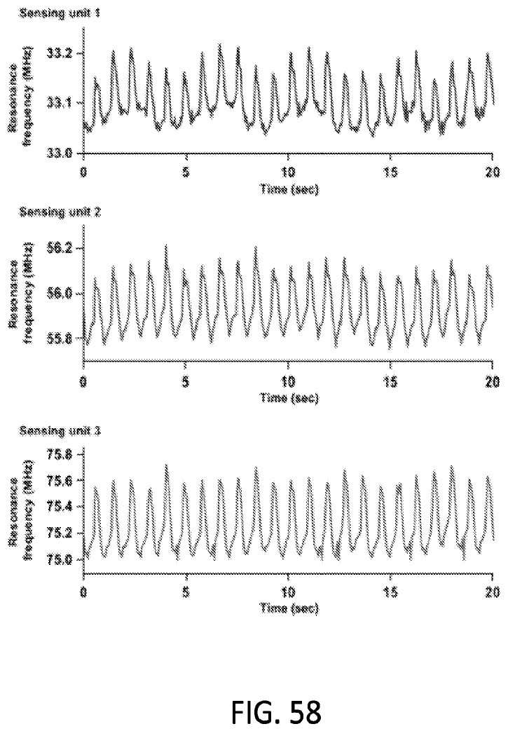

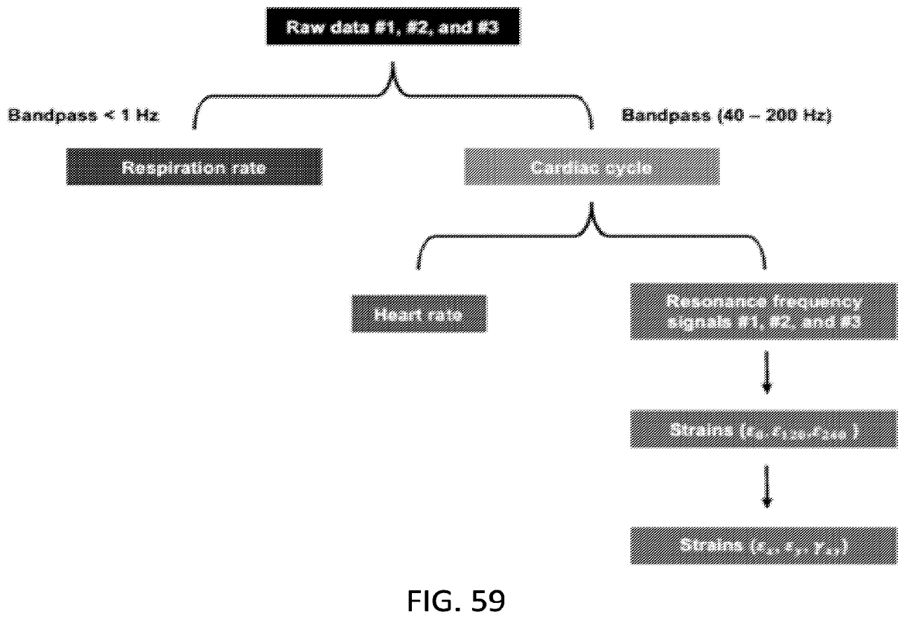

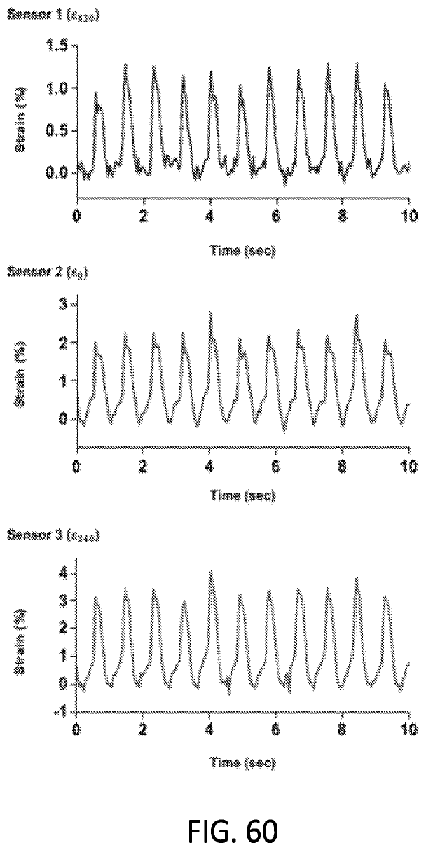

In vivo evaluations utilize the left ventricle (LV) of the heart in an ovine model. FIG. 57 shows in vivo acute evaluations in an ovine model according to certain embodiments of the invention. The multi-sensing element mounts on the LV, and the wireless module lies in the dorsal subcutaneous area, with the electrical connections supported by a flexible cable, as shown in FIG. 57(a). Specifically, in the device designed for measurements on the LV of an ovine animal as shown in FIG. 57(a), the multi-sensing element integrates on the LV, and the wireless module lies in the dorsal subcutaneous area. A flexible cable provides an electrical interface. FIG. 57(b) presents the raw data from one sensing unit in a representative segment (duration: 20 s; raw data for the three units appear in FIG. 58). FIG. 59 shows data filtering for raw data measured from the LV of the porcine heart according to certain embodiments of the invention, where data with a bandpass of <1 Hz determines the respiration rate, and data with a bandpass of 40-200 Hz determines the heart rate and the strains. Filtering the data in different cutoff frequencies as shown in FIG. 59 yields the respiration rate (duration: ˜4-5 s; marked red) and heart rate (duration: ˜0.9 s; marked blue) of the anesthetized ovine animal, as shown in FIG. 57(c). FIG. 57(d)-(f) shows quantitative assessment of principal strains (d) εₓ and (e) ε_{y} and shear strain (f) γ_{xy} of the ovine LV. FIG. 60 shows quantitative assessment of the strains of the ovine LV along the three arms corresponding to FIG. 57(d)-(f). Analyzing the data from the three sensing units together through coordinate transformation results in principal strains (εₓ and ε_{y}) and shear strain (γ_{xy}) as shown in FIG. 60 and FIG. 57(d)-(f).

The results described here span topics in laser-material interactions, fabrication sequences, material layouts, device architectures, and system-level demonstrations of eco/bioresorbable electronics through ex vivo and in vivo studies, all in the context of scanned, picosecond-pulsed laser ablation approach to manufacturing. Systematic characterization illustrates compatibility with the full range of eco/bioresorbable materials, with the ability for precise thickness control, high spatial resolution, and good overlay registration in multi-layered configurations. Successful fabrication of silicon MOSFETs and diodes suggests applicability to nearly all classes of IC and

CMOS components, in isolation or as interconnected collections. Comprehensive evaluations in animal models demonstrate the strong performance of a diverse collection of bioresorbable electronic devices across multiple organs, ranging from wireless physiological monitors in tissue cavities to microcapillary flow sensing probes in transplanted tissues, biopotential sensing arrays for the cerebral cortex, and wireless strain systems for the epicardial surface. Because this dry manufacturing process aligns well with established tools adapted from the flexible printed circuit board industry, it is possible to envision use at industrial scales. These concepts may form a starting point for further developments of laser-based schemes for miniaturized, high-density, and stacked IC and microelectromechanical systems (MEMS) chips in eco/bioresorbable forms and high-resolution feature size, particularly when used in conjunction with previously reported schemes for transfer printing of eco/bioresorbable components produced using foundry facilities. Additional improvements may include increasing the resolution and overlay registration through advanced optics, e.g., flat-topped laser spots, and mechanical stages, introducing fast switching of laser wavelengths selected to match the absorption spectra of processed materials, incorporating in-line sensing for closed-loop control of manufacturing processes, and extending applications to include multi-layer patterning directly on biomaterials or living tissues.

The inventors have performed experiments to apply the methods as described in the embodiments above, which are further elaborated in details as follows.

### Materials

Materials used in the experiments included bioresorbable substrates, conductors, semiconductors, dielectrics, and encapsulants. Polymer substrates include cellulose acetate (CA; thickness: 35 μm; Goodfellow Corporation, PA, USA), poly (lactic acid) (PLA; thickness: 50 μm; Goodfellow Corporation, PA, USA), and poly(D,L-lactide-co-glycolide) (PLGA; lactide:glycolide: 65:35; M_{w}: 40-75 k; thickness: ˜10 μm; Sigma-Aldrich, MO, USA). Electron beam evaporation (AJA International Inc., MA, USA) yielded uniform layers of metal (materials: Mg, Zn, and Fe) on these substrates. A bioresorbable thermoplastic polymer (BTP; synthesis process appears in the following section) enabled robust lamination of uniform metal films (thickness: 5-250 μm; materials: Mg, Zn, Mo, and Fe; Goodfellow Corporation, PA, USA) on these substrates. Device-grade, monocrystalline silicon membranes (thickness: 0.5-2 μm) were obtained from silicon on insulator wafers (SOI wafers; University Wafer, Inc., MA, USA) by eliminating the buried oxide layer via immersion in hydrofluoric acid (HF; concentration: 49%; Honeywell, NC, USA) for 2 d. These membranes were doped by ion implantation (Leonard Kroko, Inc., CA, USA) or diffusion (Ty star Corporation, CA, USA) in patterns and at levels to match device requirements. Annealing by a rapid thermal processor (RTP; AW-610; Allwin 21 Corporation, CA, USA) preceded immersion in HF to eliminate the buried oxide. Uniform layers of dielectric materials, such as SiO₂, (thickness: 10-100 nm), were deposited by electron beam evaporation (AJA International Inc., MA, USA).

### Synthesis

Chemical synthesis procedures yielded BTP and WPU polymers. The synthesis of BTP followed a previously reported strategy based on a thiol-yne step-growth polymerization⁶⁶ Briefly, two monomers, bis(3-mercaptopropyl) succinate (C_{SS}; Sigma-Aldrich, MO, USA) and 1,3-propane diyl dipropiolate (C_{3A}; Sigma-Aldrich, MO, USA), were synthesized by sulfuric acid-catalyzed Fischer esterification reactions in toluene. Css was synthesized from 3-mercaptopropanol (25.000 g, 271 mmol, 2.14 equiv; Sigma-Aldrich, MO, USA) and succinic acid (15.000 g, 144 mmol, 1 equiv; Sigma-Aldrich, MO, USA). C_{3A} was synthesized from propanediol (20.000 g, 263 mmol, 1 equiv; Sigma-Aldrich, MO, USA) and propiolic acid (50.000 g, 714 mmol, 2.7 equiv; Sigma-Aldrich, MO, USA). All three monomers, C_{SS} (0.715 g, 0.2 equiv), C_{3A} (2.420 g, 1 equiv), and 1,10-decanedithiol (C_{10S}; 2.210 g, 0.8 equiv; TCI America, Inc., OR, USA) were distilled prior to addition to an oven dried round bottom flask charged with HPLC-grade chloroform (CHCl₃; 25 mL; Thermo-Fisher Scientific, MA, USA). The resulting solution was cooled to −15° C. while stirring for 15 min before adding 1,8-diazabicyclo(5.4.0)undec-7-ene (DBU; 20 μL; Sigma-Aldrich, MO, USA) and then warming to room temperature 5 min after addition. The reaction proceeded for 2 hr then a slight excess of C_{3A} (1-2 drops) was introduced to cap terminating thiol moieties. Shortly after, butylated hydroxytoluene (BHT; 0.225 g; Sigma-Aldrich, MO, USA) was added to quench DBU, then the solution was precipitated into diethyl ether (400 mL; Sigma-Aldrich, MO, USA) and filtered to give a yellow polymer which was subsequently dried under high vacuum at ambient temperature for 24 hr. Size-exclusion chromatography (SEC; EcoSEC HLC-8320GPC; Tosoh Bioscience LLC, PA, USA) and differential scanning calorimetry (DSC; Q2000 DSC Packing; TA Instruments, DE, USA) determined the molecular weight (Mₙ=32.5 kDa; M_{w}=68.2 kDa; Ð_{M}=2.1) and the melting temperature (Tₘ=99.3° C.), respectively. Fabrication of uniform layers of BTP utilized compression molding (temperature at 120° C. and compression at 22 kN for 10 min, 44 kN for 10 min, and 66 kN for 60 min, respectively), with a polyimide (PI) mold (thickness=10-200 μm) treated by anti-adhesion spray to achieve target thicknesses. The synthesis of WPU followed previous protocols⁶², modified by adding a natural wax mixture (ratio of Candelilla wax to Beeswax: 4:1) to enhance the water barrier property. In general, the synthesis began with melting polycaprolactone triol (PCL-triol; Mₙ: ˜900 g/mol; 2.7 g; Sigma-Aldrich, MO, USA) at 60° C., followed by mixing with hexamethylene diisocyanate (HDI; 504 μL; Sigma-Aldrich, MO, USA) and butyl acetate (15 mL; Sigma-Aldrich, MO, USA) until full dissolution at the same temperature. Adding tin (II) 2-ethylhexanoate (Sn (Oct)₂; Sigma-Aldrich, MO, USA) after cooling the solution to room temperature and then drop-casting the resulting solution on a hydrophobic glass surface yielded films with the desired thickness (˜100 μm) defined by the volume and area.

### Tool and Processing Modes for Laser Ablation

The ablation processes described here used a commercial picosecond-pulsed laser system (wavelength: 1030 nm; pulse duration: 1.0±0.2 ps; beam diameter: 15 μm; maximum processing area: 30 cm×23 cm; LPKF Laser & Electronics, OR, USA). The average power (80-200 mW), scanning speed (200-600 mm/s), frequency (40-200 kHz), and the number of repetitions were adjusted according to different application scenarios. Grid distance (2-7 μm) and grid mode (X-parallel, Y-parallel, and XY-parallel) were adjusted to meet patterning requirements. The ablation process enabled by this laser allowed material thinning, complete removal, and cutting by appropriate selection of these parameters. Thinning corresponds to controlled, patterned reductions in the thickness of the top layer to a target value. Removal corresponds to complete, patterned ablation of the top layer with minimum damage to the underlying materials. Multi-layered device architectures were fabricated by multiple cycles of material preparation-ablation. Cutting corresponded to ablation through the entire material stack, including the substrate, to achieve target shapes.

### Characterization of the Ablation Process

### Ablated thickness

Square regions of monocrystalline Si (5 mm×5 mm) were ablated using various laser settings (average power, scanning speed, frequency, and the number of repetitions) at fixed grid distance (d; 7 μm) and grid mode (XY-parallel). Atomic force microscopy (AFM; Dimension Icon; tapping mode; Bruker, MA, USA) defined the topography of the Si surfaces to determine the ablated thicknesses.

### Lateral Resolution

Ablation (average power: 90 mW; scanning speed: 300 mm/s; frequency: 200 Hz; number of repetitions: 3 for ribbon shape, 5 for trench shape; grid distance: 2 μm; grid mold: Y-parallel) with different projected widths between two adjacent laser scans determined ribbon (projected width: 7-23 μm) and trench (projected width: 15-23 μm) shapes in monocrystalline Si or Mg. Specifically, ribbon shapes (length: 1 mm) were created by separating two parallel ablated regions with well-controlled projected widths. Trench shapes were achieved by ablating various single line segments (length: 1 mm) with different projected widths. AFM revealed the cross-sectional profiles of the Si ribbons and trench shapes to determine the peak heights and effective widths (integrated cross-sectional area divided by the peak height). Optical profilometry (Dektak 150 Surface Profiler; Veeco, NY, USA) characterized the cross-sectional profiles of the Mg ribbons and trenches.

### Overlay Registration

The top layer (material: Au; thickness: 1 μm) on a polymer substrate (material: polytetrafluoroethylene; thickness: 350 μm) was ablated into a crisscross pattern (length and width: 8 mm; line width: 0.5 mm; average power: 100 mW; frequency: 100 kHz; scanning speed: 500 mm s⁻¹; number of repetitions: 1; grid distance: 7 μm; grid mold: XY-parallel), with three alignment marks (circle shape; diameter: 1.5 mm) on the corners. After changing the position of the sample, the alignment marks were used to perform aligned ablation of a second, smaller crisscross pattern (length and width: 6 mm; line width: 0.5 mm) on the top layer with the same central point as the first. The designed distance between the two cross patterns was 0.5 mm. An optical microscope characterized the actual distance. The difference between these distances corresponds to the overlay registration along the x- and y-axes.

### Minimal Damage to Underlying Substrate

Characterization involved studies of damage to a PLA film (thickness: 50 μm) when ablating a top layer of Si (thickness: 500 nm) and damage to a Si membrane (thickness: 500 nm) when ablating a top layer of Mg (thickness: 300 nm). In both cases, after completely removing the top layer by ablation in a square pattern (5 mm×5 mm), the samples were characterized using a 3D laser scanning microscope (OLS 5000; Olympus Corp., Tokyo, Japan) to obtain the thickness of the ablated surface region T_{ab}. The difference between T_{ab} and the thickness of top layer (Tₜₒₚ) represented a useful metric to define the selectivity of the ablation process to the top layer.

### Line Edge Roughness

Characterization involved studies of Si (thickness: 2 μm; width: 100 μm) and Mg (thickness: 500 nm; width: 100 μm) ribbons patterned on PLA substrates (thickness: 50 μm). Top-view imaging the ribbons through a digital microscope (VHX-7000 series; Keyence, IL, USA), followed by stripes analysis in ImageJ, determined the RMS line edge roughness.

### Surface Treatment on Substrates

Ablation also enabled surface texturing of polymer substrates for bioresorbable electronics. As an example, ablation defined a square pattern array (dimension: 60 μm; space: 60 μm) to generate a micro-textured surface with a Cassie-Baxter state and enhanced surface hydrophobicity.

### FEA Studies of Heat Transfer

Analysis focused on energy absorbed from laser radiation using the transient heat transfer module of Abaqus (Dassault Systems Simulia Corp., RI, USA). The sample consisted of a 500-nm-thick Si MM on a 50-μm-thick PLA substrate. The absorbed energy was modeled by a body heat flux Q in the Si at the region of the laser spot (diameter d₀=15 μm). The heat flux was uniform along the thickness direction and followed a Gaussian distribution⁵¹ (Q =Qₘₐₓ exp(−50r²d₀⁻²)). The thermal conductivity (k) and thermal diffusivity (α) were k_{Si}=148 W m⁻¹ K⁻¹ and α_{Si}=88.0 mm² s⁻¹ for Si MM, respectively, and k_{PLA}=0.13 W m⁻¹ K⁻¹ and α_{PLA}=0.058 mm² s⁻¹ for PLA substrate, respectively.

### Laser Ablation for Wireless Bioresorbable Physiological Monitors

### Inductors

Bonding a uniform foil of Mg (thickness: 280 μm) on a PLA substrate (thickness: 50 μm) using the BTP interfacial adhesive (5 wt % in chloroform; heat compression for 30 min) defined samples for processing. Ablation (average power: 4 W; frequency: 40 kHz; scanning speed: 400 mm s⁻¹; number of repetitions: 4; grid distance: 7 μm; grid mold: XY-parallel) created a thinned central circular area of the Mg layer (thickness: from 280 μm to 190 μm) to define the bottom electrode of the device. Another ablation process (average power: 4 W; frequency: 40 kHz; scanning speed: 400 mm s⁻¹; number of repetitions: 125; grid distance: 7 μm; grid mold: XY-parallel) applied to the surrounding area of the Mg created the necessary helical coil pattern (line width: 150 μm; line space: 500 μm; out diameter: 10 mm; number of turns: 6; thickness of 280 μm). Measurements using a 3D laser scanning microscope (OLS 5000) characterized the surface topography of the device.

### Other Components

Ablation defined the geometries of all components, including a circular top electrode (PLGA/Zn/PLGA; thickness: 10/2/10 μm), an annular spacer (PLA; thickness: 50 μm), and BTP encapsulation (top, thickness: 25 μm; bottom, thickness: 200 μm) by cutting. Manual assembly of the system began with adhering the circular top electrode (PLGA/Zn/PLGA) onto the top BTP encapsulation (thickness: 25 μm) using hexane to soften the surface of the BTP. Adhering the annular spacer (PLA) and the inductor with the thinned bottom electrode (Mg/PLA) to the device in sequence at 70° C. using a conductive wax (a mixture of W microparticles and Candelilla wax at a weight ratio of 16:1) connected the top and bottom electrodes. Sealing the device with a bottom encapsulating layer of BTP (thickness: 200 μm) at 120° C. completed the assembly process.

### Evaluations of Wireless Bioresorbable Physiological Monitors in a Rat Model

The animal studies were performed according to protocols approved by the institutional animal care and use committees at Washington University in St Louis and conformed to the Guide for the Care and Use of Laboratory Animals. Male Lewis rats weighing 250-350 g (Charles River Laboratories, MA, USA) received hair trimming, povidone-iodine spreading (10 vol %), and isopropanol spreading (70 vol %) in the surgical area, followed by subcutaneous injections of buprenorphine hydrochloride (0.05 mg/kg; Reckitt Benckiser Healthcare) and ampicillin (50 mg/kg; Sage Pharmaceuticals) for pain management and infection prevention at the implantation site, respectively. The surgeons provided appropriate postoperative care along with analgesia post-surgery. Device implantation involved a surgical process of anesthetizing the rats with isoflurane gas, holding the head in a stereotaxic frame, opening a craniectomy and dura, placing the bioresorbable device on the cortical surface, and sealing the craniectomy with a commercial dental cement on the edge of the device (Micron Superior Type 2; Prevest Denpro Limited, Jammu, India). Embedding a clinical ICP monitor (Camino System; Model MPM-1; Integra LifeSciences) in a nearby craniectomy enabled comparison testing as a reference. Carefully squeezing and releasing the flank of rats induced increments and recoveries in ICP values, respectively. The readout system included a single turn readout coil that was connected to a vector network analyzer (E5063A; Keysight, CA, USA). The external readout coil was placed on top of the head without physical contact, with a fixed working distance of ˜1 cm. The vector network analyzer measured the real and imaginary parts of S-matrix element S₁₁ in real time. The measurements of intracranial pressure, respiration rate, and heart rate were performed under animal anesthesia.

### Laser Ablation for Bioresorbable Microvascular Flow Sensing Probes

Vacuum deposition of a uniform Mg layer (thickness: 180 nm) on a PLA substrate (thickness: 50 μm) completed the preparation step. Ablation (average power: 150 mW; frequency: 100 kHz; scanning speed: 400 mm s⁻¹; number of repetitions: 9; grid distance: 7 μm; grid mold: XY-parallel) patterned the Mg into four serpentine shapes to define one resistive heater (trace width: 50 μm; spacing: 75 μm; resistance: ˜350Ω) and three thermistors (trace width: 18 μm; spacing: 30 μm; resistance: ˜650Ω), each with a different distance to the heater (1.8, 2.9, and 10.0 mm, respectively). Ablation then cut the PLA substrate into a needle-shaped structure (dimensions: 30×3.5 mm) to complete the fabrication (average power: 100 mW; frequency: 100kHz; scanning speed: 400 mm s⁻¹; number of repetitions: 60; grid distance: 7 μm; grid mold: XY-parallel). The resistive heater filled the area at the tip of the needle to ensure a spatially uniform pattern of heating. Manual assembly of the device began with bonding a uniform layer of PLGA adhesive (thickness: ˜10 μm) on top of the probe at 70° C. (over the glass transition temperature (T_{g}) of PLGA (˜65° C.)). Encapsulating the probe on top and bottom with films of BTP (thickness: 25 μm) and sealing the edges using a soldering iron at 120° C. for 2 min, completed the assembly.

### Characterization of Bioresorbable Microvascular Flow Sensing Probes

The characterization included studies of the thermal distributions produced by the resistive heater and of the sensitivity of the thermistors. A programmable low noise power source (6221; Keithley Instruments, OH, USA) supplied current to the heater with a power of 1-48 mW. An infrared (IR) camera (a6255sc; FLIR Systems, OR, USA) imaged the temperature distribution across the device, including the temperature values at the positions of the three thermistors. Simultaneously, a data acquisition/switch unit (34970A/34901A; Agilent Technologies, CA, USA) determined the resistances of these thermistors. The slope of the plot of resistance as a function of temperature determined the sensitivity of these thermistors, and enabled conversion from resistance to temperature.

### Evaluations of Bioresorbable Microvascular Flow Sensing Probes in a Porcine Model

The animal studies were performed according to protocols approved by the institutional animal care and use committees at Washington University in St Louis and conformed to the Guide for the Care and Use of Laboratory Animals. Four live pigs were utilized in separate experiments for this study. Anesthesia was induced with Telazol, ketamine, and xylazine followed by maintenance with inhaled isoflurane. Pedicled rectus abdominus myocutaneous flaps were raised with the superficial superior epigastric vein, and the deep superior epigastric artery and veins were separated for occlusion. An incision along muscle fibers near the bottom surface of the rectus abdominus muscle was made by a 15 #blade, followed by the probe deployment into the muscle pocket. After restoring the flap to its anatomic orientation, a commercial ViOptix device which was mounted onto the skin padder, with wire connection to an external monitor, recorded the oxygen saturation (StO₂) of the flap as the reference. A voltage source (arbitrary waveform generator; 3390; Keithley Instruments, OH, USA) powered the heater and the data acquisition/switch unit (34970A/34901A) measured the resistance values of the three thermistors. A complete process of in vivo evaluation included ischemia (I; 15 min), recovery (R; 15 min), congestion (C; 15 min), and recovery (R; 15 min). An Acland clamp was applied to the right deep superior epigastric artery, which led to a complete ischemia state (I). The Acland clamps were applied to the deep and superficial superior epigastric veins to induce a venous congestion state (C). Releasing the Acland clamps from artery or veins allowed for flap recovery (R) and re-establishment of a stable baseline reading. After the completion of the experiments, the animals were euthanized with pentobarbital.

### Laser Ablation for Bioresorbable Multi-Layered Si-Based Electrode Arrays

The process consisted of two cycles of material preparation-ablation and one cycle of cutting. Ablation (1^{st} ablation; average power: 90 mW; frequency: 200 kHz; scanning speed: 300 mm s⁻¹; number of repetitions: 25; grid distance: 6 μm; grid mold: X-parallel) patterned the doped Si MM (thickness: 2 μm) on the PLA substrate (thickness: 50 μm) to define the sensing element. Three alignment makers (diameters: 1.5 mm) were fabricated on the corners of the Si pattern. Ablation patterned a Mg layer (thickness: 1 μm) deposited by electron beam evaporation (AJA International Inc., MA, USA), into the shape of connection traces (2^{nd} ablation; average power: 30 mW; frequency: 200 kHz; scanning speed: 400 mm s⁻¹; number of repetitions: 45; grid distance: 6 μm; grid mold: X-parallel). Ablation cut the PLA substrate into a ribbon shape to complete the fabrication (average power: 1 W; frequency: 100 kHz; scanning speed: 400 mm s⁻¹; number of repetitions: 80; grid distance: 7 μm; grid mold: XY-parallel). Characterization of the electrochemical impedance spectrum (impedance and phase angle) of these electrodes used an Autolab electrochemical workstation (Metrohm AG, Herisau, Switzerland) in 0.1 M PBS (pH 7.4) at room temperature.

### Evaluations of Bioresorbable Multi-Layered Si-Based Electrode Arrays in a Mouse Model

The animal studies were performed according to protocols approved by the institutional animal care and use committees at Northwestern University and conformed to the Guide for the Care and Use of Laboratory Animals. Young adult male C57BL/6 mice (postnatal day: 60-80;weight: ˜20 g) were used. Mice were maintained at ˜25° C. and humidity ranging from 30 to 70%, on a standard 12 h light-12 h dark cycle (lights on at 6:00 am) with ad libitum feeding. Right primary motor cortex (M2) was targeted for stereotactic injection under isoflurane anesthesia using the following coordinates relative to bregma: AP=+0.8 mm, ML=+1.5 mm and DV=−1.0 mm. AAV9-syn-ChrimsonR-tdT virus (Addgene #59171) was diluted to a titer of ˜5×10¹² GC mL⁻¹ in PBS (pH 7.4) and injected using a glass pipette and a micro-injector. Three weeks after virus transduction, the local skull was removed to create a 3×3 mm² cranial window for acute ECoG measurement using the multi-layered Si-based electrode array. The electrode was placed over the right primary motor cortex and a ground/reference electrode was placed at AP=+3.0 mm, ML=+0.5 mm. For optogenetic activation of ChrimsonR, an external LED driver coupled with a commutator (Plexon, Dallas, TX) provided light stimulation (wavelength: 570 nm; power density: ˜10 mW mm⁻²). The LED emission terminal was placed approximately 1 mm over the device. The device was connected to an electrophysiology data acquisition system (RHD recording system; Intan Technologies, CA, USA) for data recording. The ECoG signal was analyzed using Open Ephys Acquisition Board and Open Ephys GUI (Open Ephys, Cambridge, MA) at 1 kHz and subsequently filtered with a bandpass filter between 0.1-20 Hz.

### Laser Ablation for Bioresorbable Multi-Layered Si-Based Semiconductor Devices

The process consisted of three cycles of material preparation-ablation. Ablation (1^{st} ablation; average power: 80 mW; frequency: 200 kHz; scanning speed: 1000 mm s⁻¹; number of repetitions: 45; grid distance: 7 μm; grid mold: Y-parallel) first directly patterned a doped Si MM (thickness: 2 μm) to define the sensing element (the doped areas differ in MOSFETs and diodes, see FIGS. 39-41). Patterns on the corners of the Si formed three alignment markers (diameters: 1.5 mm). Thermal oxidation (Tytan mini furnace system; Tystar Corporation, CA, USA) and plasma-enhanced chemical vapor deposition (PECVD; STS LpX CVD; SPTS Technologies, Newport, UK) formed uniform layers of SiO₂ (thickness: 100 nm) and SiNx (thickness: 200 nm) for MOSFETs and diodes, respectively, subsequently patterned by ablation (MOSFETs: average power: 70 mW; frequency: 200 kHz; scanning speed: 1000 mm s⁻¹; number of repetitions: 20; grid distance: 7 μm; grid mold: Y-parallel; diodes: average power: 70 mW; frequency: 200 kHz; scanning speed: 1000 mm s⁻¹; number of repetitions: 10; grid distance: 7 μm; grid mold: Y-parallel) to form several vias for connection. Ablation patterned a layer of Mg deposited by electron beam evaporation (thickness: 50 nm for MOSFETs and 100 nm for diodes) into the shape of connection pads (dimension: 160×580 μm; average power: 30 mW; frequency: 200 kHz; scanning speed: 1000 mm s⁻¹; number of repetitions: 6 for MOSFETs and 29 for diodes; grid distance: 2 μm for MOSFETs and 7 μm for diodes; grid mold: Y-parallel for MOSFETs and XY-parallel for diodes). Characterization utilized a manual probe station (S-1160; Signatone, CA, USA) with a semiconductor parameter analyzer (4155C; Keysight, CA, USA). A white LED (10,000 lux) for basic measurements of the photoresponses of the diodes.

### Laser Ablation for Wireless Bioresorbable Cardiac Systems

### Multi-Sensing Element

This element included several components, from bottom to top: a uniform bottom encapsulating layer of WPU (thickness: ˜100 μm; dimensions of each arm: 15.0×5.0 mm), a tri-arm Y-shaped layer with three pairs of bottom electrodes (Zn/CA; thickness: 2/35 μm; dimensions of each arm: 6.9×3.1 mm), three single-arm floating electrodes (Zn/CA; thickness: 2/35 μm; dimensions: 10.9×3.1 mm), a tri-arm Y-shaped top cover (CA; thickness: 35 μm; dimensions of each arm: 6.9×3.1 mm), and a top encapsulating layer WPU (thickness: ˜100 μm; dimensions of each arm: 15.0×5.0 mm). The ablation process patterned all of these components. For the bottom electrodes, the floating electrodes, and the top cover, ablation directly patterned the top Zn layer (thickness: 2 μm; vacuum deposition through electron beam evaporation) on the CA substrate (thickness: 35 μm). Ablation cut the WPU (tri-arm; dimensions of each arm: 15.0×5.0 mm) into desired shapes. After stacking these components, bonding the edge of the top and bottom WPU encapsulation layers by heat compression at 200° C. (bonding width at edge: ˜0.9 mm) completed the fabrication.

### Flexible Cable

The flexible cable included an array with six independent traces (material: Zn/CA; thickness: 5/35 μm) and a uniform layer of PLGA as encapsulation (thickness: ˜10 μm). For the traces, the ablation process directly patterned the top Zn layer (number: 6; trace width: 1 mm; trace space: 1.5 mm; length: 120 mm) on the CA substrate (dimensions: 120 mm×14 mm). Laser cutting of a uniform layer of PLGA defined the top encapsulation (dimensions: 120 mm×14 mm). Heat compression at 70° C. facilitated the bonding of the encapsulation. Long cables utilized Zn wires (diameter: 250 μm; Goodfellow Corporation, PA, USA) sandwiched by uniform layers of PLGA using heat compression at 70° C., as an extended connection between the flexible cable and the wireless module.

### Wireless Module

The wireless module included three in-plane inductors (Zn/PLA; thickness: 25/50 μm) with different inductance values, encapsulated by two uniform layers of PLA (thickness: 50 μm). Ablation defined all of the components. For in-plane inductors, BTP served as an adhesive between the Zn film (thickness: 25 μm) and the PLA substrate (thickness: 50 μm). Ablation patterned the top Zn layer (thickness: 25 μm) into a helical coil shape (line width: 200 μm; line space: 500 μm; outside diameter: 19.6 mm; number of turns: 4, 6, and 10). Ablation cut the PLA encapsulation layer (diameter: 90 mm) and the substrate (diameter: 22.6 mm) into the desired shapes. The three inductors were each positioned between PLA encapsulation layers to achieve optimized Q factors, defined by measurements of S₁₁, when evaluated together using a single-turn readout coil (diameter: ˜5 mm) connected to a vector network analyzer (E5063A; Keysight, CA, USA). Tightly bonding these components together by spreading a small amount of chloroform and heat compression at 120° C. in sequence completed the fabrication.

### Connection Among the Modules

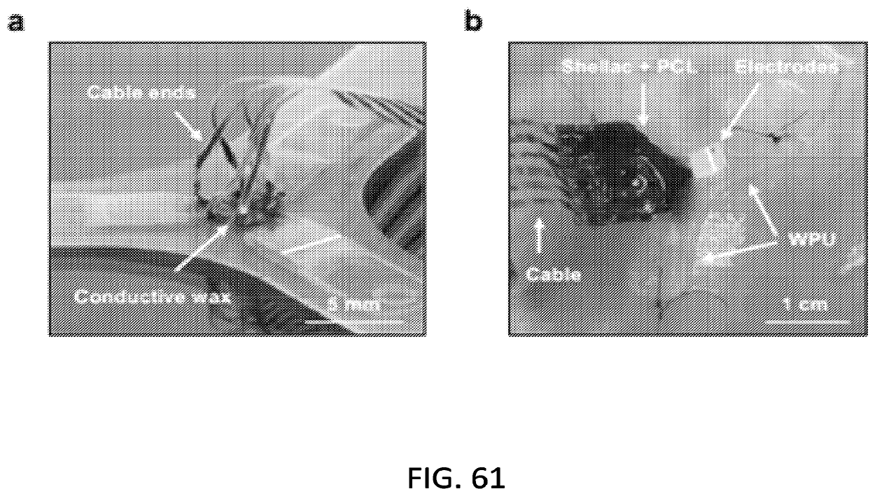

FIG. 61 shows connection between the multi-sensing element and the flexible cable according to certain embodiments of the invention, where (a) shows bioresorbable conductive wax connects the ends of the six independent traces in the flexible cable to the six pads of the three pairs of bottom electrodes in the multi-sensing element, and (b) shows a bioresorbable polymer mixture based on PCL and shellac (ratio of PCL to shellac: 9:1) seals the connection sites, and the PCL provides encapsulation and the shellac provides adhesion. Connections between the multi-sensing element and the flexible cable used six independent traces interfaced to the six pads of the bottom electrodes in the multi-sensing element using bioresorbable conductive wax, as shown in FIG. 61(a). To suppress the parasitic capacitance in the cable, the sequence of the six independent traces followed the order of bottom electrode pair #1-#2-#3-#1-#2-#3. In this way, the distance between the two traces for the same bottom electrode pair was controlled to ˜6.5 mm. A bioresorbable polymer mixture based on PCL and shellac (ratio of PCL to shellac: 9:1) sealed the connection sites, as shown in FIG. 61(b). For connection between the flexible cable and the wireless module, zinc wires (diameter: 250 μm; Goodfellow Corporation, PA, USA) bonded to the contact pads in the flexible cable and in the wireless module with a bioresorbable conductive wax. Encapsulation used a natural wax mixture (ratio of Candelilla wax to Beeswax: 4:1).

### FEA Analysis of Wireless Bioresorbable Cardiac Systems

FEA performed through a commercial software (Abaqus; Dassault Systemes Simulia Corp., RI, USA), predicted the mechanical deformation and the electrical-to-mechanical coupling of the cardiac device. The bottom electrodes, floating electrodes, and top cover were modeled by 4-node shell elements, while the encapsulations and the connection sites were modeled by 8-node solid elements. Refined mesh with a size smaller than 5% of the electrode width was adopted to ensure accuracy. Two models were adopted for the cardiac tissues—the simplified model and the Living Heart Model. In the simplified model, the heart geometry was simplified as a cuboid (thickness: 10 mm), and the in-plane size was much larger than the device size. The materials of the cardiac tissues adopted the hyperelastic constitutive relationship, and the heart contraction was modeled via an equal biaxial stretching with uniform far-field strain. FIG. 52 and FIG. 54A utilized the simplified model. As a more accurate model, the Living Heart Model (Dassault Systemes Simulia Corp., RI, USA), which was adopted and integrated with the device, including the precise geometries from 3D scanning, the anisotropic and strain-hardening constitutive relationship, and the electrical-to-mechanical coupled heart contraction. FIG. 44 and FIG. 54B utilized the Living Heart Model.

### Characterization of the Wireless Bioresorbable Cardiac System

Characterization tests evaluated the strain sensitivity, bending sensitivity and effects of mechanical constraints on cardiac tissues through ex vivo evaluations. A vector network analyzer (E5063A) measured the S11 antenna parameter (reflection coefficient) of the three LC-type devices together in one system to determine the resonance frequency and Q factor of each. Device performance under stretching (strain: 0-10%) and bending (angle: 0-90°) was determined through the use of a custom mechanical stretcher. Quantitative imaging of a collection of black plastic particles (diameter: 1 mm) spread on the porcine heart revealed the effects of mechanical constraints imposed by the device on underlying cardiac tissues. 3D-particle tracking velocimetry (3D-PTV) through two digital cameras (2,560×1,600 pixels CMOS Phantom Miro 340 with 12

GB on-board memory and frame rates of 1,000 f.p.s.) recorded the location of each particle as a function time during simulated cycles of beating. Pre-processing, calibration, 3D reconstruction, tracking, and post-processing exploited previous 3D PT codes. Tracking the 3D reconstructed positions of these particles determined the deformation of the porcine hearts without and with the cardiac device, and with a rigid film for comparison. Ex vivo studies utilized porcine hearts with similar structures and dimensions to those of humans. An artificial heart system with a mechanical pump provided an adjustable and oscillatory pattern of flow (TS410 tubing module; Transonic Systems Inc., NY, USA) and pressure (Tru-wave disposable pressure transducer; Edwards Lifesciences, CA, USA) in the right ventricle (RV) of the porcine heart. Recordings of the deformation using the device mounted on the RV with three sutures on the end of each arm utilized the readout coil of the vector network analyzer (distance between the wireless module and the readout coil: ˜5 mm).

### Animal Evaluations of Wireless Bioresorbable Cardiac Systems

The animal procedures were performed as per U.S. Department of Agriculture Animal Welfare Regulations at an accredited facility. Ovine model was utilized for this study. The multi-sensing element was mounted on the left ventricle (LV) of the heart, with the wireless module in the dorsal subcutaneous area. During the mounting process, a custom 3D-printed accessory grasped all three arms to prevent the device from bending or fracturing. The device was firmly attached to the LV with three suturing sites between the holes of each arm and the underlying tissues. The device continuously recorded the cardiac signals through a wireless readout coil connected to a vector network analyzer (E5063A). The signals were analyzed using a custom program (LabVIEW; National Instruments, TX, USA).

Yet a further aspect of the invention relates to a bioresorbable electronic device formed by the method as discussed above, or an electronic apparatus having a multi-layer bioresorbable electronic device formed by the method discussed above.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### REFERENCE LIST

1. Hwang, S.-W. et al. A Physically Transient Form of Silicon Electronics. Science (1979) 337, 1640-1644 (2012).
2. Li, C. et al. Design of biodegradable, implantable devices towards clinical translation. Nat Rev Mater 5, 61-81 (2020).
3. Han, W. B., Lee, J. H., Shin, J. & Hwang, S. Advanced Materials and Systems for Biodegradable, Transient Electronics. Advanced Materials 32, 2002211 (2020).
4. Hsu, E., Barmak, K., West, A. C. & Park, A.-H. A. Advancements in the treatment and processing of electronic waste with sustainability: a review of metal extraction and recovery technologies. Green Chemistry 21, 919-936 (2019).
5. Irimia-Vladu, M., Glowacki, E. D., Sariciftci, N. S. & Bauer, S. Green Materials for Electronics. (Wiley-VCH, 2017).
6. Global Pacemakers and Implantable Cardioverter Defibrillators (ICDs) Market-Analysis and Forecast, 2017-2023. (2017).
7. Brain Monitoring Systems Market By Product (fNIRS, EEG, MEG, MRI), By Modalit y(Portable/Handheld, Standalone), By End User (Hospitals, Ambulatory Surgical Centers, Clinics, Pediatric & Neonatal Intensive Care Units) & Region—Forecast 2022-2028. (2022).
8. Earle, N. The Future of IoT. (2022).
9. Das, R., Chang, Y.-H. & Dyson, M. RFID Forecasts, Players and Opportunities 2022-2032. (2021).
10. Kang, S.-K. et al. Bioresorbable silicon electronic sensors for the brain. Nature 530, 71-76 (2016).
11. Yu, K. J. et al. Bioresorbable silicon electronics for transient spatiotemporal mapping of electrical activity from the cerebral cortex. Nat Mater 15, 782-791 (2016).
12. Bai, W. et al. Bioresorbable photonic devices for the spectroscopic characterization of physiological status and neural activity. Nat Biomed Eng 3, 644-654 (2019).
13. Koo, J. et al. Wireless bioresorbable electronic system enables sustained nonpharmacological neuroregenerative therapy. Nat Med 24, 1830-1836 (2018).
14. Choi, Y. S. et al. Fully implantable and bioresorbable cardiac pacemakers without leads or batteries. Nat Biotechnol 39, 1228-1238 (2021).
15. Koo, J. et al. Wirelessly controlled, bioresorbable drug delivery device with active valves that exploit electrochemically triggered crevice corrosion. Sci Adv 6, (2020).
16. Boutry, C. M. et al. Biodegradable and flexible arterial-pulse sensor for the wireless monitoring of blood flow. Nat Biomed Eng 3, 47-57 (2019).
17. Jamshidi, R., Taghavimehr, M., Chen, Y., Hashemi, N. & Montazami, R. Transient Electronics as Sustainable Systems: From Fundamentals to Applications. Adv Sustain Syst 6, 2100057 (2022).
18. Hwang, S.-W. et al. High-Performance Biodegradable/Transient Electronics on Biodegradable Polymers. Advanced Materials 26, 3905-3911 (2014).
19. Yu, X., Shou, W., Mahajan, B. K., Huang, X. & Pan, H. Materials, Processes, and Facile Manufacturing for Bioresorbable Electronics: A Review. Advanced Materials 30, 1707624 (2018).
20. Shou, W. et al. Low-Cost Manufacturing of Bioresorbable Conductors by Evaporation—Condensation—Mediated Laser Printing and Sintering of Zn Nanoparticles. Advanced Materials 29, 1700172 (2017).
21. Lee, Y. K. et al. Room Temperature Electrochemical Sintering of Zn Microparticles and Its Use in Printable Conducting Inks for Bioresorbable Electronics. Advanced Materials 29, 1702665 (2017).
22. Han, W. B., Ko, G.-J., Shin, J.-W. & Hwang, S.-W. Advanced manufacturing for transient electronics. MRS Bull 45, 113-120 (2020).
23. Huang, X. et al. Biodegradable Materials for Multilayer Transient Printed Circuit Boards. Advanced Materials 26, 7371-7377 (2014).
24. MAIMAN, T. H. Stimulated Optical Radiation in Ruby. Nature 187, 493-494 (1960).
25. Joe, D. J. et al. Laser-Material Interactions for Flexible Applications. Advanced Materials 29, 1606586 (2017).
26. Munoz-Garcia, C. et al. Influence of wavelength and pulse duration on the selective laser ablation of WOx, VOx and MoOx thin films. Surfaces and Interfaces 28, 101613 (2022).
27. Sundaram, S. K. & Mazur, E. Inducing and probing non-thermal transitions in 27 semiconductors using femtosecond laser pulses. Nat Mater 1, 217-224 (2002).
28. Hermann, S., Harder, N.-P., Brendel, R., Herzog, D. & Haferkamp, H. Picosecond laser ablation of SiO2 layers on silicon substrates. Applied Physics A 99, 151-158 (2010).
29. Park, J. H. et al. Self-powered flexible electronics beyond thermal limits. Nano Energy 56, 531-546 (2019).
30. Keller, W. J. et al. Physics of picosecond pulse laser ablation. J Appl Phys 125, 085103(2019).
31. Mustafa, H., Matthews, D. T. A. & Römer, G. R. B. E. Wavelength dependence of picosecond-pulsed laser ablation of hot-dip galvanized steel. Applied Physics A 128, 296 (2022).
32. Vidal, F. et al. Critical-Point Phase Separation in Laser Ablation of Conductors. Phys Rev Lett 86, 2573-2576 (2001).
33. von der Linde, D. & Sokolowski-Tinten, K. The physical mechanisms of short-pulse laser ablation. Appl Surf Sci 154-155, 1-10 (2000).
34. von der Linde, D. & Schüler, H. Breakdown threshold and plasma formation in femtosecond laser-solid interaction. Journal of the Optical Society of America B 13, 216 (1996).
35. Shin, G. et al. Flexible Near-Field Wireless Optoelectronics as Subdermal Implants for Broad Applications in Optogenetics. Neuron 93, 509-521.e3 (2017).
36 Bian, J. et al. Laser Transfer, Printing, and Assembly Techniques for Flexible Electronics. Adv Electron Mater 5, 1800900 (2019).
37. Kim, K. K., Choi, J., Kim, J., Nam, S. & Ko, S. H. Evolvable Skin Electronics by In Situ and In Operando Adaptation. Adv Funct Mater 2106329 (2021) doi: 10.1002/adfm.202106329.
38. Yang, Y. et al. A laser-engraved wearable sensor for sensitive detection of uric acid and tyrosine in sweat. Nat Biotechnol 38, 217-224 (2020).
39. Wang, M., Yang, Y. & Gao, W. Laser-engraved graphene for flexible and wearable electronics. Trends Chem 3, 969-981 (2021).
40. You, R. et al. Laser Fabrication of Graphene-Based Flexible Electronics. Advanced Materials 32, 1901981 (2020).
41. Stuart, T. et al. Biosymbiotic, personalized, and digitally manufactured wireless devices for indefinite collection of high-fidelity biosignals. Sci Adv 7, (2021).
42. Chichkov, B. N., Momma, C., Nolte, S., Alvensleben, F. & Tünnermann, A. Femtosecond, picosecond and nanosecond laser ablation of solids. Appl Phys A Mater Sci Process 63,109-115 (1996).
43. Kumar, V., Verma, R., Kango, S. & Sharma, V. S. Recent progresses and applications in laser-based surface texturing systems. Mater Today Commun 26, 101736 (2021).
44. Rogers, J. A., Someya, T. & Huang, Y. Materials and Mechanics for Stretchable Electronics. Science (1979) 327, 1603-1607 (2010).
45. Kim, D.-H., Ghaffari, R., Lu, N. & Rogers, J. A. Flexible and Stretchable Electronics for Biointegrated Devices. Annu Rev Biomed Eng 14, 113-128 (2012).
46. Wang, Y., Shen, N., Befekadu, G. K. & Pasiliao, C. L. Modeling pulsed laser ablation of aluminum with finite element analysis considering material moving front. Int J Heat Mass Transf 113, 1246-1253 (2017).
47. Pou-Alvarez, P. et al. Nanosecond, picosecond and femtosecond laser surface treatment of magnesium alloy: role of pulse length. Surf Coat Technol 427, 127802 (2021).
48. Bogaerts, A. & Chen, Z. Effect of laser parameters on laser ablation and laser-induced plasma formation: A numerical modeling investigation. Spectrochim Acta Part B At Spectrosc 60, 1280-1307 (2005).
49. Neuenschwander, B. et al. Processing of metals and dielectric materials with ps-laserpulses: results, strategies, limitations and needs. in (eds. Niino, H., Meunier, M., Gu, B. & Hennig, G.) 75840R (2010). doi: 10.1117/12.846521.
50. Liu, H. C., Mao, X. L., Yoo, J. H. & Russo, R. E. Early phase laser induced plasma diagnostics and mass removal during single-pulse laser ablation of silicon. Spectrochim Acta Part B At Spectrosc 54, 1607-1624 (1999).
51. Xian, J. et al. A Simple Model to Predict Machined Depth and Surface Profile for Picosecond Laser Surface Texturing. Applied Sciences 8, 2111 (2018).
52. Shin, J. et al. Bioresorbable pressure sensors protected with thermally grown silicon dioxide for the monitoring of chronic diseases and healing processes. Nat Biomed Eng 3, 37-46 (2019).
53. Yang, Q. et al. Materials, Mechanics Designs, and Bioresorbable Multisensor Platforms for Pressure Monitoring in the Intracranial Space. Adv Funct Mater 30, 1910718 (2020).
54. Smit, J. M., Zeebregts, C. J. & Acosta, R. Timing of Presentation of the First Signs of Vascular Compromise Dictates the Salvage Outcome of Free Flap Transfers. Plast Reconstr Surg 122, 991-992 (2008).
55. Chao, A. H., Meyerson, J., Povoski, S. P. & Kocak, E. A review of devices used in the monitoring of microvascular free tissue transfers. Expert Rev Med Devices 10, 649-660 (2013).
56. Lee, Y. K. et al. Dissolution of Monocrystalline Silicon Nanomembranes and Their Use as Encapsulation Layers and Electrical Interfaces in Water-Soluble Electronics. ACS Nano 11, 12562-12572 (2017).
57. Oskam, G., Long, J. G., Natarajan, A. & Searson, P. C. Electrochemical deposition of metals onto silicon. J Phys D Appl Phys 31, 1927-1949 (1998).
58. Wang, S. et al. Large-Area Free-Standing Ultrathin Single-Crystal Silicon as Processable Materials. Nano Lett 13, (2013).
59. Hwang, S.-W. et al. Materials and Fabrication Processes for Transient and Bioresorbable High-Performance Electronics. Adv Funct Mater 23, 4087-4093 (2013).
60. Chang, J.-K. et al. Materials and processing approaches for foundry-compatible transient electronics. Proceedings of the National Academy of Sciences 114, E5522-E5529 (2017).
61. Dandel, M., Lehmkuhl, H., Knosalla, C., Suramelashvili, N. & Hetzer, R. Strain and Strain 61 Rate Imaging by Echocardiography-Basic Concepts and Clinical Applicability. Curr Cardiol Rev 5, 133-148 (2009).
62. Choi, Y. S. et al. Stretchable, dynamic covalent polymers for soft, long-lived bioresorbable electronic stimulators designed to facilitate neuromuscular regeneration. Nat Commun 11, 5990 (2020).
63. Ferraiuoli, P. et al. Full-field analysis of epicardial strain in an in vitro porcine heart platform. J Mech Behav Biomed Mater 91, 294-300 (2019).
64. Zhang, H., Iijima, K., Huang, J., Walcott, G. P. & Rogers, J. M. Optical Mapping of Membrane Potential and Epicardial Deformation in Beating Hearts. Biophys J 111, 438-451 (2016).
65. Yin, L., Bozler, C., Harburg, D. v., Omenetto, F. & Rogers, J. A. Materials and fabrication sequences for water soluble silicon integrated circuits at the 90 nm node. Appl Phys Lett 106, 014105 (2015).
66. Wandel, M. B. et al. Concomitant control of mechanical properties and degradation in resorbable elastomer-like materials using stereochemistry and stoichiometry for soft tissue engineering. Nat Commun 12, 446 (2021).
67. Schneider, C. A., Rasband, W. S. & Eliceiri, K. W. NIH Image to ImageJ: 25 years of image analysis. Nat Methods 9, 671-675 (2012).
68. Schindelin, J. et al. Fiji: an open-source platform for biological-image analysis. Nat Methods 9, 676-682 (2012).
69. Lee, P. T. et al. Left Ventricular Wall Thickness and the Presence of Asymmetric Hypertrophy in Healthy Young Army Recruits. Circ Cardiovasc Imaging 6, 262-267 (2013).
70. SIMULIA LIVING HEART https://www.3ds.com/products-services/simulia/solutions/life-sciences-healthcare/living-heart-human-model/.
71. Kim, J.-T., Nam, J., Shen, S., Lee, C. & Chamorro, L. P. On the dynamics of air bubbles in Rayleigh-Bénard convection. J Fluid Mech 891, A7 (2020).

What is claimed is:

1. A method for performing laser structuring of multi-layered ecoresorbable or bioresorbable materials, comprising:
sequentially forming a plurality of ecoresorbable or bioresorbable material layers on a flexible substrate;
patterning, locally thinning or ablating, using a picosecond-pulsed laser system, the ecoresorbable or bioresorbable material layers; and
patterning and ablating, using the picosecond-pulsed laser system, the flexible substrate.

2. The method of claim 1, wherein the flexible substrate is a flexible biodegradable polymeric substrate.

3. The method of claim 2, wherein the flexible substrate is formed by polylactic acid (PLA) or cellulose acetate (CA).

4. The method of claim 1, wherein the material layers are formed on the flexible substrate by physical lamination, transfer printing, deposition or growth techniques.

5. The method of claim 1, wherein the picosecond-pulsed laser system is operated with a wavelength of about 1030 nm, a pulse duration of about 1.0 picoseconds, and a beam diameter of 15 μm.

6. The method of claim 1, wherein the material layers include a semiconductor layer and a metal layer.

7. The method of claim 6, wherein the semiconductor layer and the metal layer are formed and patterned by:
forming the semiconductor layer on the flexible substrate;
patterning, locally thinning or ablating, using the picosecond-pulsed laser system, the semiconductor layer;
after patterning, locally thinning or ablating the semiconductor layer, forming the metal layer on the flexible substrate and the patterned semiconductor layer; and
patterning, locally thinning or ablating, using the picosecond-pulsed laser system, the metal layer.

8. The method of claim 7, further comprising:
after patterning, locally thinning or ablating the semiconductor layer and prior to forming the metal layer, forming at least one alignment marker on the semiconductor layer to align the patterned semiconductor layer and the metal layer.

9. The method of claim 1, wherein a patterned structure of the ecoresorbable or bioresorbable material layers has a resolution of about 5-10 μm and alignment accuracy of less than 5 μm.

10. A method for fabricating a multi-layered bioresorbable electronic device, comprising:
sequentially forming a plurality of ecoresorbable or bioresorbable material layers on a flexible substrate; and
patterning, locally thinning or ablating, using a picosecond-pulsed laser system, the ecoresorbable or bioresorbable material layers to form at least one sensing component and interconnection traces of the bioresorbable electronic device.

11. The method of claim 10, wherein the flexible substrate is a flexible biodegradable polymeric substrate.

12. The method of claim 11, wherein the flexible substrate is formed by polylactic acid (PLA) or cellulose acetate (CA).

13. The method of claim 10, wherein the material layers are formed on the flexible substrate by physical lamination, transfer printing, deposition or growth techniques.

14. The method of claim 10, wherein the picosecond-pulsed laser system is operated with a wavelength of about 1030 nm, a pulse duration of about 1.0 picoseconds, and a beam diameter of 15 μm.

15. The method of claim 10, wherein the bioresorbable electronic device is a bi-layered electronic device, and the material layers include a semiconductor layer and a metal layer.

16. The method of claim 15, wherein the semiconductor layer and the metal layer are formed and patterned by:
forming the semiconductor layer on the flexible substrate;
patterning, locally thinning or ablating, using the picosecond-pulsed laser system, the semiconductor layer to form the at least one sensing component of the bioresorbable electronic device;
after patterning, locally thinning or ablating the semiconductor layer, forming the metal layer on the flexible substrate and the patterned semiconductor layer; and
patterning, locally thinning or ablating, using the picosecond-pulsed laser system, the metal layer to form the interconnection traces of the bioresorbable electronic device.

17. The method of claim 16, further comprising:
after patterning, locally thinning or ablating the semiconductor layer and prior to forming the metal layer, forming at least one alignment marker on the semiconductor layer to align the patterned semiconductor layer and the metal layer.

18. The method of claim 10, wherein the bioresorbable electronic device is a multi-layered electronic device, and the material layers includes a first sensing layer and a plurality of second sensing layers.

19. The method of claim 18, wherein the first sensing layer and the second sensing layers are formed and patterned by:
forming the first sensing layer on the flexible substrate;
patterning, locally thinning or ablating, using the picosecond-pulsed laser system, the first sensing layer; and
after patterning the first sensing layer, sequentially forming and patterning, locally thinning or ablating each of the second sensing layers by:
forming a respective second sensing layer on the flexible biodegradable polymer substrate and the patterned first sensing layer; and
patterning, locally thinning or ablating, using the picosecond-pulsed laser system, the respective second sensing layer.

20. The method of claim 18, wherein the first sensing layer and the second sensing layers include at least one semiconductor layer and at least one metal layer.

21. The method of claim 20, further comprising:
after patterning, locally thinning or ablating the semiconductor layer and prior to forming the metal layer, forming at least one alignment marker on the semiconductor layer to align the patterned semiconductor layer and the metal layer.

22. The method of claim 10, further comprising:
patterning and ablating, using the picosecond-pulsed laser system, the flexible substrate around the at least one sensing component and the connection traces to form at least one stretchable portion of the bioresorbable electronic device.

23. The method of claim 10, having a manufacture time of about 30 minutes.

24. A bioresorbable electronic device formed by the method of claim 10.

25. The bioresorbable electronic device of claim 24, having a resolution of about 5-10 μm and alignment accuracy of less than 5 μm.

26. An electronic apparatus having a bioresorbable electronic device formed by the method of claim 10.

27. A method for fabricating a multi-layered bioresorbable electronic device, comprising:
forming a first material layer on a flexible substrate, wherein the first material layer is ecoresorbable or bioresorbable;
patterning, locally thinning or ablating, using a picosecond-pulsed laser system, the first material layer to form at least one sensing component of the bioresorbable electronic device;
after patterning, locally thinning or ablating the first material layer, forming a second material layer on the flexible substrate and the patterned first material layer, wherein the second material layer is ecoresorbable or bioresorbable; and
patterning, locally thinning or ablating, using the picosecond-pulsed laser system, the second material layer to form interconnection traces of the bioresorbable electronic device.

28. The method of claim 27, wherein the flexible substrate is a flexible biodegradable polymeric substrate.

29. The method of claim 28, wherein the flexible substrate is formed by polylactic acid (PLA) or cellulose acetate (CA).

30. The method of claim 27, wherein the first material layer and the second material layer are respectively formed by physical lamination, transfer printing, deposition or growth techniques.

31. The method of claim 27, wherein the picosecond-pulsed laser system is operated with a wavelength of about 1030 nm, a pulse duration of about 1.0 picoseconds, and a beam diameter of 15 μm.

32. The method of claim 27, wherein the first material layer is a semiconductor layer, and the second material layer is a metal layer.

33. The method of claim 27, further comprising:
after patterning, locally thinning or ablating the first material layer and prior to forming the second material layer, forming at least one alignment marker on the first material layer to align the patterned first material layer and the second material layer.

34. The method of claim 27, further comprising:
patterning and ablating, using the picosecond-pulsed laser system, the flexible substrate around the at least one sensing component and the connection traces to form at least one stretchable portion of the bioresorbable electronic device.

35. The method of claim 27, having a manufacture time of about 30 minutes.

36. A bioresorbable electronic device formed by the method of claim 27.

37. The bioresorbable electronic device of claim 36, having a resolution of about 5-10 μm and alignment accuracy of less than 5 μm.

38. An electronic apparatus having a bioresorbable electronic device formed by the method of claim 27.
